(19) United States
(12) Patent Application Publication
(10) Pub. No.: US 2016/0271149 A1
(43) Pub. Date: Sep. 22, 2016

(54) THERAPEUTIC COMPOUNDS THAT SUPPRESS PROTEIN ARGININE METHYLTRANSFERASE ACTIVITY FOR REDUCING TUMOR CELL PROLIFERATION

(71) Applicant: Epinova Therapeutics Corp., Mendham, NJ (US)

(72) Inventor: Dalton Einhorn, Mendham, NJ (US)

(21) Appl. No.: 15/071,321

(22) Filed: Mar. 16, 2016

Related U.S. Application Data
(60) Provisional application No. 62/133,573, filed on Mar. 16, 2015.

(51) Int. Cl. A61K 31/575; A61K 31/42; A61K 31/195; A61K 31/4439; A61K 31/155

(52) U.S. Cl. CPC A61K 31/575; A61K 31/4439; A61K 31/155; A61K 31/195; A61K 31/42

(57) ABSTRACT

The described invention provides methods for modulating gene expression of a gene related to proliferation of a population of tumor cells. The method entails administering a therapeutic amount of a therapeutic compound to a cell, a tissue, or a mammal, wherein the therapeutic amount of the therapeutic compound is effective to suppress methyltransferase activity of a protein arginine methyltransferase. Modulation of the protein arginine methyltransferase activity in turn modulates methylation of a target protein that affects gene expression of the gene, and may suppress the proliferation of the population of tumor cells.

### CROSS-REFERENCE TO RELATED APPLICATIONS

This application claims the benefit of priority to U.S. Provisional Application No. 62/133,573, filed Mar. 16, 2015, the content of which is incorporated by reference herein in its entirety.

### FIELD OF THE INVENTION

The described invention relates to epigenetic modifications of gene expression, therapeutic compounds that modulate those epigenetic modifications, and cancer.

### BACKGROUND OF THE INVENTION

Epigenetic Modifications of Gene Expression

Mammalian gene expression is regulated at multiple epigenetic layers. The four major layers are DNA methylation patterns, histone modification signatures, chromatin conformation characteristics, and non-coding RNAs. Zhang, G. and Pradhan, S. “Mammalian Epigenetic Mechanisms,” IUMB Life, 66(4): 240-56 (2014).

DNA Methylation

DNA methylation by DNA cytosine-5 methyltransferases and accessory proteins is primarily thought to control gene expression by (a) changing transcription factor binding affinity to a gene promoter; (b) by affecting the binding of methylation-specific recognition factors to promotor or gene bodies; or (c) by altering the chromatin structure and spatial accessibility of transcription factors and/or other DNA binding proteins. Id. Modification of DNA may repel binding of certain proteins while attracting others, e.g., methyl-CpG-binding domain (MBD) containing proteins, e.g., MeCP2, MBD1, MBD2, MBD3, and MBD4, which recognize and bind methylated CpG sequences and act as insulators for transcription factor binding. Id. Some studies report that methylation leads to compact the DNA, thereby making transcriptional activity at the methylated gene. Other studies indicate that DNA methylation affects core histone modification and linker histone mobility but not chromatin condensation in bulk chromatin and heterochromatin. Id. There is increasing evidence that a host of proteins are essential in the DNA methylation process in mammalian cells. Jones, P. A. and Liang, G., “Rethinking how DNA methylation patterns are maintained,” Nat. Rev. Genet. 10: 805-11 (2009).

Demethylation of DNA has been described to occur by passive dilution and by dynamic removal of 5 methylcytosine. In the passive demethylation pathway, a gradual loss of 5 methylcytosine by inhibition of DNMTs, leading to a gradual loss of 5 methylcytosine (5mC) during successive cell divisions. Several mechanisms have been proposed for active demethylation, including base excision repair by Gadd45 and other companion proteins, removal of 5mC by thymidine DNA glycosylase (TDG), and 5mC deamination by activation-induced deaminase/apolipoprotein B mRNA-editing enzyme complex (AID/APOBEC) followed by consequent mismatch repair. Zhang, G. and Pradhan, S. “Mammalian Epigenetic Mechanisms,” IUMB Life, 66(4): 240-56 (2014). 5mC can be oxidized in an iterative manner by the Ten eleven translocation (TET) dioxygenase family of enzymes. 5mC is oxidized to 5-hydroxymethylcytosine (5hmC) which is oxidized to 5-formylcytosine (5fC), and then to 5-carboxylcytosine (5caC) (Ito, S. et al., “Tet proteins can convert 5-methylcystosine to 5-formylcytosine and 5-carboxylcytosine,” Science 333 (6047): 1300-1303 (2011). 5caC is removed by thymine DNA glycosylase and base excision repair (BER) pathway enzymes. Zhang, G. and Pradhan, S. “Mammalian Epigenetic Mechanisms,” IUMB Life, 66(4): 240-56 (2014).

DNA methylation contributes to mammalian development, X-chromosome inactivation and genome stability. Failure to maintain correct methylation patterns leads to aberrant DNA methylation often observed in human diseases, including neurodevelopmental defects, neurodegenerative, neurological and autoimmune diseases, and cancers. Lv, J. et al, “DiseaseMeth: a human disease methylation database,” Nucleic Acid Res. 40: D1030-D1035 (2012).

Histone Modification

Nucleosomes are the basic unit of chromatin that constitute the bulk of compacted chromosomes. The assembly of nucleosomes as well as compaction of nucleosomal arrays into higher-order chromatin structures create a highly restrictive environment for nuclear processes that require access to DNA. The packaging of eukaryotic DNA into nucleosomes inhibits the access of factors to DNA and thus results in the repression of transcription, replication, and recombination. To counterbalance the repressive nature of chromatin, a variety of chromatin remodeling factors use the energy of ATP hydrolysis to facilitate the interaction of proteins with nucleosomal DNA. ATP dependent chromatin remodeling complexes are characterized by the presence of an ATPase subunit from SNF2-like family of the DEAD/H (SF2) DNA-stimulated ATP-ases. The highly conservative hSWI/SNF multisubunit complexes contain hBRM or BRG1 ATPases which alter the histone-DNA contacts enabling the access of general transcription factors to promoter regions. Remodeling complexes are targeted to promoters via interactions with sequence-specific transcription factors. [From http://www.biocarta.com/pathfiles/h_hSWI-SNFpathway.asp citing Narlikar G J et al. Cooperation between complexes that regulate chromatin structure and transcription. Cell. 2002 108(4):475-87; Vignali M et al. ATP-dependent chromatin-remodeling complexes. Mol Cell Biol. 2000 (6):1899-910]

A mononucleosome consists of genomic DNA wrapped around histone octamer scaffolds. The protruded NH₂ tails of histones in the nucleosomes can be modified, for example, by lysine and arginine methylation, lysine acetylation, serine, threonine and tyrosine phosphorylation, and lysine ubiquitination and sumoylation. Jenuwein, T. and Allis, C D, “Translating the histone code,” Sci. 293: 1074-80 (2001). These modifications contribute to the epigenetic regulation of gene expression, i.e., they can change the charge of chromatin, leading to a more condensed or open state, which dictates the accessibility of regulatory proteins, including transcriptional regulators. In transcriptionally non-permissive chromatin, regulatory repressor proteins recognize modified histone tails for recruitment, leading to chromatin condensation and obstruction of transcriptional activator binding.

Many of these modifying enzymes also target other cellular proteins. For example, the histone H3K4 methyltransferase SET7/9 also catalyzes the methylation of various non-histone proteins, including DNMT1, p53, yes-associated protein (Yap), SUV39H1, nuclear hormone estrogen receptor alpha (ERα), Zhang, G. and Pradhan, S., “Mammalian Epigenetic Mechanisms,” IUBMB Life 66(4): 240-256 (2014).

Mammalian genomes are compacted into highly condensed chromatin via histones and other scaffold proteins to maintain a compartmentalized three dimensional conformation with genes organized into hubs of different transcription states. Handoko, L., et al, “CTCF-mediated functional chromatin interactions in pluripotent cells,” Nat. Genet. 43: 630-38 (2011).

It is well established that histone modification represents a fundamental step in regulating DNA accessibility during various cellular processes such as transcription, replication and DNA repair [Jenuwein, T. and Allis, D; “Translating the histone code,” Science 293: 1074-80 (2001); Strahl, B D and Allis, C D, “The language of covalent histone modifications,” Nature 403: 41-45 (2000)]. Conversion of chromatin from a repressed form to a more open and active state and vice versa relies on the coordinated interplay between histone-modifying enzymes, ATP-dependent chromatin remodelers, and DNA-modifying enzymes.

Histones undergo a variety of post-translational modifications in their globular domains and N-terminal tails, and among the known histone modifications, lysine acetylation and methylation have been the most studied [Berger, S L, “Histone modifications in transcriptional regulation,” Current Op. Genetics & Devel., 12: 142-48 (2002)].

Different types of histone modifications function in a combinatorial fashion to fine-tune nuclear events. Using multiple histone modifications, a cell can integrate different cellular signaling pathways at the chromatin level. The complex histone modification network functions in four steps: (1) modifying triggering histone residues; (2) recognition of trigger modification; (3) modifying consequent histone residues, and (4) recruitment of effector complexes. [Zhang, G, and Pradhan, S, “Mammalian Epigenetic Mechanisms,” IUBMB Life 66(4): 240-56 (2014) citing Siganuma, T and Workman, J L, “Signals and Combinatorial functions of Histone Modifications,” Ann. Rev. Biochem. 80: 473-99 (2011)].

Histone Acetylation

Acetylation of the lysine ε-amino group, first discovered on histones, is a dynamic posttranslational modification (PTM) regulated by the opposing activities of lysine acetyltransferases (KATs) and histone deacetylases (HDACs). Delcluve, G P, et al, “Role of histone deacetylases in epigenetic regulation: emerging paradigms from studies with inhibitors,” Clinical Epigenetics 4: 5 (2012).

Histone acetylation is a modulator of chromatin structure involved in DNA replication, DNA repair, hetero-chromatin silencing and gene transcription [Id; citing Groth, A., et al., “Chromatin Challenges during DNA replication and repair,” Cell 128: 721-33 (2007); Shahbazian, M D et al., “Functions of site-specific histone acetylation and deacetylation,” Ann. Rev. Biochem. 76: 75-100 (2007)]. Hyperacetylation contributes to the decondensed chromatin state and maintains the unfolded structure of the transcribed nucleosome [Id; citing Shahbazian, M D et al., “Functions of site-specific histone acetylation and deacetylation,” Ann. Rev. Biochem. 76: 75-100 (2007); Tse, C., et al, “Disruption of higher order folding by core histone acetylation dramatically enhances transcription of nucleosomal arrays by RNA polymerase III,” Molec. Cell Biol. 18: 4629-38 (1998); Wang, X, et al, “Effects of histone acetylation on the solubility and folding of the chromatin fiber,” J. Biol. Chem. 276: 12764-68 (2001); Shogren, K M, et al, “Histone H4-K16 acetylation controls chromatin structure and protein interactions,” Science 311: 844-847 (2006); Davie, J R, et al, “Nuclear Organization and chromatin dynamics: Sp1, Sp3, and histone deacetylases,” Adv. Enzyme Regul. 48: 189-238 (2008)]. Specific acetylated sites on core histones are read by bromodomain modules found in proteins, and sometimes in KATs, which are components of chromatin-remodeling complexes involved in transcriptional activation [Id; citing Lee, K K, Workman, J L, “Histone acetyltransferase complexes: one size doesn't fit all,” Nat. Rev. Molec. Cell Biol. 8: 284-95 (2007)].

Lysine acetylation, which is implicated in the regulation of nearly all nuclear functions and many cytoplasmic processes [Id; citing Choudhary, C. et al, “Lysine acetylation targets protein complexes and co-regulates major cellular functions,” Science 325: 834-40 (2009)], is regulated by and/or regulates other PTMs. Through either recruitment or occlusion of binding proteins, PTMs may lead to or prevent a secondary PTM on histones and nonhistone proteins [Id, citing Latham, J A, and Dent, S Y, “Cross-regulation of histone modifications,” Nat. Struct. Molec. Biol. 14: 1017-24 (2007); Yang, X J and Seto E, “Lysine Acetylation codified cross talk with other posttranslational modifications,” Molec. Cell 31: 449-61 (2008)]. In particular, histone H3 phosphorylation on serine 10 or 28, rapid and transient PTMs in response to the stimulation of signaling pathways such as the mitogen-activated protein kinase (MAPK) pathways, are associated with histone acetylation and transcriptional activation of specific genes [Id citing Hazzalin, C A and Mahadevan, L C, “MAPK-regulated transcription: a continuously variable gene switch?”, Nat. Rev. Molec. Cell Biol. 3: 30-40 (2002)]. A cross-talk also exists between histone acetylation and H3 methylation. Although acetylation is generally linked to transcription activation, the effect of methylation depends on which amino acid residue is modified and the degree to which this residue is methylated (mono-, di- or trimethylation of lysine). Methylation of H3 lysine 4 or 36 is associated with transcription activation, but methylation of lysine 9 or 27 is linked to transcription repression [Id., citing Kouzarides, T., “Chromatin modifications and their function,” Cell 128: 693-705 (2007); Allis, C D et al, “New nomenclature for chromatin-modifying enzymes,” Cell 131: 633-36 (2007)].

HDACs

To date, 18 different mammalian HDACs have been identified and divided into four classes based on their sequence similarity to yeast counterparts [Id., citing de Ruijter, A J et al, “Histone deacetylases (HDACs): characterization of the classical HDAC family,” Biochem. J. 370: 737-49 (2003); Gregoretti, I V et al, “Molecular evolution of the histone deacetylase family: functional implications of phylogenetic analysis,” J. Mol. Biol. 338: 17-31 (2004)]. HDACs from the classical family are dependent on Zn2+ for deacetylase activity and constitute classes I, II and IV.

Class I HDACs, closely related to yeast RPD3, comprise HDAC1, HDAC2, HDAC3 and HDAC8. Class I HDACs are ubiquitously expressed nuclear enzymes, although HDAC8 is generally poorly expressed [Id., citing de Ruijter, A J et al, “Histone deacetylases (HDACs): characterization of the classical HDAC family,” Biochem. J. 370: 737-49 (2003)]. Except for HDAC8, class I HDACs are components of multiprotein complexes.

The HDAC1 and HDAC2 homo- or heterodimer can exist with different proteins. The combination of these proteins likely determines the overall activity, substrate specificity and genomic location of the HDAC1 and/or HDAC2 containing complex.

Knockout studies have shown that class I HDACs are involved in cell proliferation and survival [Id., citing Marks, P A, “Histone deacetylase inhibitors: a chemical genetics approach to understanding cellular functions<” Biochim. Biophys. Acta 1799: 717-25 (2010); Haberland, M. et al, “The many roles of histone deacetylases in development and physiology: implications for disease and therapy,” Nat. Rev. Genet. 10: 32-42 (2009)]. HDAC1 and HDAC2 form homo- and heterodimers between each other [Id citing Taplick, J et al, “Homo-oligomerisation and nuclear localization of mouse histone deacetylase 1,” J Molec. Biol. 308: 27-38 (2001); Luo, Y et al, “Transregulation of histone deacetylase activities through acetylation,” J. Biol. Chem. 284: 34901-34910 (2009)], which presumably allows them to act together or separately from each other. The dimer is a requirement for HDAC activity [Id, citing Luo, Y et al, “Transregulation of histone deacetylase activities through acetylation,” J. Biol. Chem. 284: 34901-34910 (2009)]. Dissociation of the dimer with a HDAC1 N-terminal peptide will inhibit HDAC activity [Id]. HDAC1 and HDAC2 are both found in multiprotein corepressor complexes Sin3, nucleosome-remodeling HDAC (NuRD) and CoREST, which are recruited to chromatin regulatory regions by transcription factors (for example, Sp1, Sp3, p53, NF-B and YY1) and have very diverse, often cell-specific, roles [de Ruijter, A J et al, “Histone deacetylases (HDACs): characterization of the classical HDAC family,” Biochem. J. 370: 737-49 (2003); Yang, X J and Seto, E, “The Rpd3/Had 1 family of lysine deacetylases from bacteria and yeast to mice and men,” Nat. Rev. Molec. Cell Biol. 9: 206-218 (2008)]. The Sin3 core complex contains Sin3A or Sin3B, HDAC1 and/or HDAC2, SAP18, SAP30 and retinoblastoma-associated proteins (RbAps) RbAp46 and RbAp48 and serves as a platform for the addition of other modules with enzymatic functions such as nucleosome remodeling, DNA methylation, histone methylation and N-acetylglucosamine transferase activity [Id citing Silverstein, R A and Ekwall, K, “Sin3: a flexible regulator of global gene expression and genome stability,” Curr. Genet. 47: 1-17 (2005); Hayakawa, T and Nakayama, J, “Physiological roles of class I HDAC complex and histone demethylase,” J. Biomed. Biotechnol. 2011: 129383 (2011)].

The NuRD complex has a variable composition that is dependent on the cell type and external stimuli. It is the only complex holding both HDAC- and ATP-dependent chromatin-remodeling activities, which are carried out by HDAC1 and/or HDAC2 and Mi-2a and/or Mi-2b, respectively. The other known components of NuRD are structural and/or regulatory proteins RbAp46/RbAp48 and, in some instances, also p66a or p66b, the methyl-CpG-binding domain-containing proteins (MBD2 or MBD3), with only MBD2 being able to recognize methylated DNA and the three members of the metastasis associated protein family (MTA1, MTA2 or MTA3), with different MTA proteins allowing distinct downstream responses to the activation of different signaling pathways [Id., citing Hayakawa, T and Nakayama, J, “Physiological roles of class I HDAC complex and histone demethylase,” J. Biomed. Biotechnol. 2011: 129383 (2011); Denslow, S A and Wade P A, “The human Mi-2/NuRD complex and gene regulation,” Oncogene 26: 5433-5438 (2007)]. Lysine-specific demethylase 1 (KDM1/LSD1) has also been identified as a component of NuRD [Id., citing Wang, Y. et al, “LS01 is a subunit of the NuRD complex and targets the metastasis program in breast cancer,” Cell 138: 660-672 (2009)].

HDAC1 and HDAC2 are also components of the Nanog- and Oct4-associated deacetylase (NODE) complex, a NuRD-related repression complex, also comprising MTA1 or MTA2, p66a or p66b, but not the histone-binding proteins RbAp46/RbAp48 and the helicase-like ATPase Mi-2. NODE is involved in the control of embryonic stem cell fate by repressing Nanog and Oct4 target genes [Id citing Liang, J. et al, “Nanog and Oct4 associate with unique transcriptional repression complexes in embryonic stem cells,” Nature Cell Biol. 10: 731-39 (2008)].

Also including HDAC1 and HDAC2, but composed of proteins distinct from those of Sin3 and NuRD, the CoREST complex is recruited by the RE1 silencing transcription (REST) factor, also known as the “neuronal restricted silencing factor” (NRSF), to the RE1 DNA motif associated with many genes encoding fundamental neuronal traits. As a component of the CoREST complex and as a consequence of histone H3 deacetylation, KDM1/LSD1 promotes demethylation of H3 dimethylated on lysine 4 (H3K4me2), an event that facilitates the formation of a repressive chromatin structure [Lee, M G et al, “An essential role for CoREST in nucleosomal histone 3 lysine 4 demethylation,” Nature 437: 432-35 (2005); Shi, Y J et al, “Regulation of LSD1 Histone demethylase activity by its associated factors,” Molec. Cell. 19: 857-64 (2005)]. Although CoREST acts as a corepressor in terminally differentiating nonneuronal cells by recruiting KDM1/LSD1 to demethylate H3K4me2 and the methyltransferase G9a to methylate H3K9 at the RE1 sites of target genes, it acts as a coactivator of transcription in embryonic stem cells and neural stem cells by recruiting an H3K4 methyltransferase to the RE1 sites of target genes [Id citing Cunliffe, V T, “Eloquent silence: developmental functions of class I histone deacetylases,” Curr. Op. Genetic Dev. 18: 404-410 (2008)]. CoREST can also form larger complexes by association with ZNF217, a Krüppel-like zinc finger protein and strong candidate oncogene product found in breast cancer, or with other complexes, such as the chromatin-remodeling complex SWI/SNF or the C-terminal binding protein (CtBP) complex [Id citing Hayakawa, T and Nakayama, J, “Physiological roles of class I HDAC complex and histone demethylase,” J. Biomed. Biotechnol. 2011: 129383 (2011); Battaglia, S. et al, “Transcription factor co-repressors in cancer biology: roles and targeting,” Intl J. Cancer 126: 2511-19 (2010)].

An HDAC complex, MiDAC, is specific to mitotic cells and includes HDAC1, HDAC2, either one of the related ELM-SANT proteins MIDEAS or TRERF1, and DNTTIP1 (terminal deoxynucleotidyl transferase (TdT)-interacting protein), although the authors who published these findings suggested that the MiDAC complex has a TdT-independent function in cell division. [Id citing Bantscheff, M. et al, “Chemoproteomics profiling of HDAC inhibitors reveals selective targeting of HDAC complexes,” Nat. Biotechnol. 29: 255-65 (2011)].

Class II HDACs, related to yeast HDA1, are divided into subclass IIa (HDAC4, HDAC5, HDAC7 and HDAC9) and subclass IIb (HDAC6 and HDAC10). Class II HDACs shuttle between the nucleus and cytoplasm and have tissue-specific expression and functions [Id citing Marks, P. A., Histone deacetylase inhibitors: a chemical genetics approach to understanding cellular functions,” Biochim. Biophys. Acta 1799: 717-25 (2010); Haberland, M. et al, “The many roles of histone deacetylases in development and physiology: implications for disease and therapy,” Nat. Rev. Genet. 10: 32-42 (2009); Yang, X J and Seto E., “The Rpd3/Hda1 family of lysine deacetylases: from bacteria and yeast to mice and men,” Nat. Rev. Molec. Cell Biol. 9: 206-18 (2008); Verdin, E et al, “Class II histone deacetylases: versatile regulators,” Trends Genet. 19: 286-93 (2003)].

Class IIa HDACs (HDAC4, HDAC5, HDAC7 and HDAC9) are signal transducers characterized by the presence in their regulatory N-terminal domains of two or three conserved serine residues subject to reversible phosphorylation. Phosphorylation leads to the binding of the 14-3-3 proteins, the nuclear export of HDACs and the derepression of their target genes. A range of kinases and phosphatases acting downstream of diverse biological pathways have been shown to regulate the nucleocytoplasmic trafficking of class IIa HDACs [Id citing Parra, M and Verdin E, “Regulatory signal transduction pathways for class IIa histone deacetylases,” Curr. Opin. Pharmacol. 10: 454-60 (2010)]. Because of a substitution of Tyr with His in their catalytic site, class IIa HDACs have negligible intrinsic deacetylase activity but are able to bind acetylated lysine. It has been suggested that, under some circumstances, class IIa HDACs may act as bromodomains, recognizing acetylated lysine in a sequence-dependent context and recruiting chromatin-modifying enzymes to regulate transcription [Id, citing Bradner, J E et al, “Chemical Phylogenetics of histone deacetylases,” Nat. Chem. Biol. 6: 238-43 (2010)]. Class IIa HDAC association with MEF2 provides additional targeting for the SMRT-NCoR complex [Id citing Fischle, W. et al, “Enzymatic activity associated with class II HDACs is dependent on a multiprotein complex containing HDAC3 and SMRT/N-CoR,” Molec. Cell 9: 45-57 (2002)]. Class IIa HDACs also interact with numerous other transcription factors. The biological relevance of these associations has been established only for the MEF2-regulated processes [Id, citing Martin M et al, “Class IIa histone deacetylases: regulating the regulators,” Oncogene 26: 5450-67 (2007); Martin M. et al, “Class IIa histone deacetylases: conducting development and differentiation,” Int. J. Dev. Biol. 53: 291-301 (2009].

Class IIb HDACs (HDAC6 and HDAC10) have duplicated catalytic domains, albeit the duplication is partial in the case of HDAC10. HDAC6 and HDAC10 shuttle between nucleus and cytoplasm, but their location is primarily cytoplasmic. Little is known of the role of HDAC10. HDAC6 is an a-tubulin deacetylase as well as a cortactin deacetylase and thus is involved in the control of microtubule- and actin-dependent cell motility. Chaperone protein HSP90 is another substrate of HDAC6. Moreover, HDAC6 plays a critical role in the cellular clearance of misfolded proteins via formation of aggresomes or autophagy [Id citing Yang, X J and Seto E, “Lysine Acetylation codified cross talk with other posttranslational modifications,” Molec. Cell 31: 449-61 (2008)].

Class IV contains only HDAC11. HDAC11 has sequence similarity to classes I and II HDACs.

Class III HDACs consist of seven sirtuins, which require the NAD+ cofactor for activity.

HDACs are upregulated in many cancers or aberrantly recruited to DNA following chromosomal translocations, particularly in hematologic malignancies [Id, citing Witt, O et al, “HDAC family: what are the cancer relevant targets?”, Cancer Lett 277: 8-21 (2009); Marks, P. A., Histone deacetylase inhibitors: a chemical genetics approach to understanding cellular functions,” Biochim. Biophys. Acta 1799: 717-25 (2010)].

Transcriptional Reprogramming by Histone Deacetylase Inhibitors

Inhibition of HDAC activity results in transcriptional reprogramming, which is believed to contribute largely to the therapeutic benefits of HDAC inhibitors on cancers, cardiovascular diseases, neurodegenerative disorders and pulmonary diseases [Haberland, M. et al, “The many roles of histone deacetylases in development and physiology: implications for disease and therapy,” Nat. Rev. Genet. 10: 32-42 (2009)]. Inhibition of HDAC enzymatic activity affects the expression of only 5% to 20% of genes, however, with equal numbers of genes being upregulated and downregulated [Id citing Smith, K T and Workman, J L; “Histone deacetylase inhibitors: anticancer compounds,” Intl J. Biochem. Cell Biol. 41: 21-25 (2009)]. Only a fraction of these changes are direct effects of HDAC inhibitors, and others are downstream effects, necessitating new protein synthesis. Only some of the direct effects can be inferred as direct consequences of inhibition of histone deacetylation, and others are the results of other mechanisms, such as the inhibition of transcription factor deacetylation, resulting in an altered affinity for DNA binding sites on target gene regulatory regions, an altered interaction with other factors or an altered half-life [Id citing Glozak, M A, et al, “Acetylation and deacetylation of non-histone proteins,” Gene 363: 15-23 (2005)]. Gene expression changes and biological functions targeted by HDAC inhibitors have been addressed in recent comprehensive reviews [Marks, P. A., Histone deacetylase inhibitors: a chemical genetics approach to understanding cellular functions,” Biochim. Biophys. Acta 1799: 717-25 (2010), Waanczyk, et al, “HDACi: going through the mechanisms,” Front. Biosci. 16: 340-59 (2011)].

BRD4

The gene bromodomain containing 4 (BRD4) is a chromatin reader protein that recognizes and binds acetylated histones and plays a key role in transmission of epigenetic memory across cell divisions and transcription regulation. It remains associated with acetylated chromatin throughout the entire cell cycle and provides epigenetic memory for postmitotic G1 gene transcription by preserving acetylated chromatin status and maintaining high-order chromatin structure. Released from chromatin upon deacetylation of histones that can be triggered by different signals such as activation of the JNK pathway or nocodazole treatment.

During interphase, BRD4 plays a key role in regulating the transcription of signal-inducible genes by associating with the P-TEFb complex and recruiting it to promoters: BRD4 is required to form the transcriptionally active P-TEFb complex by displacing negative regulators such as HEXIM1 and 7SKsnRNA complex from P-TEFb, thereby transforming it into an active form that can then phosphorylate the C-terminal domain (CTD) of RNA polymerase II. BRD4 promotes phosphorylation of ‘Ser-2’ of the C-terminal domain (CTD) of RNA polymerase II. It has been reported that BRD4 acts directly as an atypical protein kinase and mediates phosphorylation of ‘Ser-2’ of the C-terminal domain (CTD) of RNA polymerase II (Devaiah, B N et al, “BRD4 is an atypical kinase that phosphorylates serine 2 of the RNA polymerase II carboxy-terminal domain,” Proc Natl Acad. Sci. USA 109(18): 6927-32 (2012). In addition to acetylated histones, BRD4 also recognizes and binds to the acetylated REIA subunit of NF-κB, leading to further recruitment of the P-TEFb complex and subsequent activation of NF-kappa-B. BRD4 also acts as a regulator of p53/TP53-mediated transcription: following phosphorylation by CK2, it is recruited to p53/TP53 specific target promoters.

Isoform B acts as a chromatin insulator in the DNA damage response pathway. It inhibits DNA damage response signaling by recruiting the condensin-2 complex to acetylated histones, leading to chromatin structure remodeling, insulating the region from DNA damage response by limiting spreading of histone H2AFX/H2A.x phosphorylation.

The protein encoded by this gene is homologous to the murine protein MCAP, which associates with chromosomes during mitosis, and to the human RING3 protein, a serine/threonine kinase. Each of these proteins contains two bromodomains, a conserved sequence motif which may be involved in chromatin targeting. This gene has been implicated as the chromosome 19 target of translocation t(15;19)(q13;p13.1), which defines an upper respiratory tract carcinoma in young people. Two alternatively spliced transcript variants have been described. [provided by RefSeq, July 2008]

A chromosomal aberration involving BRD4 is found in a rare, aggressive, and lethal carcinoma arising in midline organs of young people. Translocation t(15;19)(q14;p13) with NUT which produces a BRD4-NUT fusion protein.

Histone Methylation

Histone methylation, which occurs on arginine, lysine and histidine amino acid residues (Zhang, G. and Pradhan, S. “Mammalian Epigenetic Mechanisms,” IUMB Life, 66(4): 240-56 (2014), is important in the regulation of chromatin and gene expression. (Dillon, S. C., “The SET-domain protein superfamily: protein lysine methyltransferases,” Genome Biology 6: 227 (2005); doi:10.1186/gb-2005-6-8-227). Mono-, di- or tri-methylation has been discovered on histone H2A, H3 and H4.

PRMTs

Members of the protein arginine methyltransferase (PRMT) family regulate chromatin structure and expression of a wide spectrum of target genes through their ability to catalyze symmetric or asymmetric methylation of histones and non-histone proteins.

Most PRMTs methylate glycine- and arginine-rich patches (GAR motifs) within their substrates. As shown in FIG. 1, arginine can be methylated in three different ways on the guanidino group: monomethylated (MMA), symmetrically dimethylated (sDMA) and asymmetrically dimethylated (aDMA). Bedford, M T, “Arginine methylation at a glance,” J. Cell Sci. 120: 4243-46 (2007). Each of the methylated arginines can induce different biological responses in the cell. Gui, S., “Substrate-induced control of product formation by protein arginine methyltransferase 1,” Biochem. 52: 199-209 (2013). It follows that controlling the type of methylation, the amount of methylation, and what proteins are methylated is necessary for a healthy cell. Id.

PRMTs use S-adenosyl-L-methionine (SAM) as a donor to transfer methyl groups to the side-chain nitrogens of arginine residues. There are three structurally defined types of S-adenosylmethionine (AdoMet)-dependent methyltransferase (Bedford, M T, “Arginine methylation at a glance,” J. Cell Sci. 120: 4243-46 (2007), citing Katz, J E et al., “Automated identification of putative methyltransferases from genomic open reading frames,” Mol. Cell Proteomics 2: 525-40 (2003)). PRMT family members fall into the largest class (Class I), which has a common seven-stranded-sheet structure. The Class II enzymes are the SET lysine methyltransferases and Class III encompasses the membrane associated methyltransferases. PRMT family members harbor a set of four conserved sequence motifs (I, post-I, II, and III) and a THW loop (Id., citing Katz, J E et al., “Automated identification of putative methyltransferases from enomic open reading frames,” Mol. Cell Proteomics 2: 525-40 (2003)). Motifs I, post-I and the THW loop form part of the AdoMet-binding pocket (Id., citing Zhang X et al., Crystal structure of the conserved core of protein arginine methyltransferase PRMT3,” EMO J. 19: 3509-19 (2000)).

SET-domain containing methyltransferases, for example, mixed-lineage leukemia-1 (MLL1) histone methyltransferase (which methylates lysine 4 of histone H3), SET1, SET7/9 (which methylates lysine 4 of histone H3) and G9a methyltransferase, (which methylates lysine 9 of histone H3), methylate the lysine residue on histone tails, whereas DOT1 family methyltransferases, for example histone methyltransferase DOT1L, methylate the globular region of histones. Zhang, G. and Pradhan, S. “Mammalian Epigenetic Mechanisms,” IUMB Life, 66(4): 240-56 (2014)

Ten mammalian PRMTs have been identified to date. Eight have been shown to catalyze the transfer of a methyl group from AdoMet to a guanidine nitrogen of arginine, generating S-adenosylhomocysteine (AdoHcy) and methylarginine. No activity has yet been demonstrated for PRMT2 and PRMT9.

PRMT1

PRMT1 is ubiquitously expressed (van Dijk, T B, et al, “Friend of PRMT1, a novel chromatin target of protein arginine methyltransferases,” Mol. Cell. Biol. 30(1): 260-72 (2010)), is the predominant mammalian type I enzyme (Id., citing Lin W J et al., “The mammalian intermediate T1S21 protein and the leukemia-associated BTG1 protein interact with a protein-arginine N-methyltransferase,” J. Biol. Chem. 271: 15034-44 (1996)); and is capable of both mono- and demethylation. (Gui, S. et al, “Substrate-induced control of product formation by protein arginine methyltransferase 1,” Biochem. 52: 199-209 (2013)). PRMT1 localizes to both the cytoplasm and the nucleus and has substrates in both these cellular compartments ((van Dijk, T B, et al, “Friend of PRMT1, a novel chromatin target of protein arginine methyltransferases,” Mol. Cell. Biol. 30(1): 260-72 (2010), citing Herrmann F et al., “Dynamics of human protein arginine methyltransferase 1 (PRMT1) in vivo,” J. Biol. Chem. 280: 38005038010 (2005)). PRMT1 methylates a number of hnRNP molecules, and this modification plays a role in the shuttling of these proteins between the cytoplasm and the nucleus (Id., citing Herrmann et al., “Arginine methylation of scaffold attachment factor A by heterogeneous nuclear ribonucleoprotein particle-associated PRMT1,” J. Biol. Chem. 279: 48774-779 (2004)). PRMT1 also methylates histone H4 at arginine 3 (Id., citing Wang et al., “Methylation of histone H4 at arginine 3 facilitating transcriptional activation by nuclear hormone receptor,” Science 293: 853-57 (2001)), thus contributing to the histone code. This modification on histone H4 functions as a transcriptional activation mark, which could either result in the recruitment of methyl-binding proteins or influence the deposition of other post-translational marks in the vicinity. As a transcriptional coactivator, PRMT1 is recruited to promoters by a number of different transcription factors (Id., citing Bedford, M T and Richard S, “Arginine methylation an emerging regulator of protein function,” Mol. Cell 18: 263-72 (2005)).

PRMT1 plays a central role as a regulator of protein function. PRMT1-knockout mice die shortly after implantation (Bedford, M T, “Arginine methylation at a glance,” J. Cell Sci. 120: 4243-46 (2007), citing Pawlak, M R et al., “Arginine N-methyltransferase 1 is required for early post-implantation mouse development, but cells deficient in the enzyme are viable,” Mol. Cell Biol. 20: 4859-69 (2000)). The crystal structure of PRMT1 in complex with the reaction product S-Adenosyl-L-homocysteine (AdoHcy) and a GAR motif has been described (Id., citing Zhang, X and Cheng, X, “Structure of the predominant protein arginine methyltransferase PRMT1 and analysis of its binding to substrate peptides,” Structure 11: 509-20 (2003)).

The majority of PRMT1 substrates are nucleic acid binding proteins that play a role in RNA processing, DNA repair, signal transduction and transcription. (van Dijk, T B, et al, “Friend of PRMT1, a novel chromatin target of protein arginine methyltransferases,” Mol. Cell. Biol. 30(1): 260-72 (2010)).

PRMT1 accounts for about 90% of global ADMA deposition. (Dhar, S. et al., “Loss of the major Type I arginine methyltransferase PRMT1 causes substrate scavenging by other PRMTs,” Scientific Reports 3: 1311/doi: 10.1038/srep01311 (2013). With the loss of PRMT1, a large number of substrates become targets for Type II and Type III PRMTs, perhaps because these substrates are no longer blocked by an ADMA modification. Id.

PRMT2

PRMT2 harbors an SRC homology 3 (SH3) domain that has at its N-terminus (Bedford, M T, “Arginine methylation at a glance,” J. Cell Sci. 120: 4243-46 (2007), citing Scott, H S et al., Identification and characterization of two putative human arginine methyltransferases (HRMT1L1 and HRMT1L2),” Genomics 48: 330-40 (1998)). Although PRMT2 does not have enzymatic activity, it does function as a coactivator for the estrogen receptor (Id., citing Qi, C et al., “Identification of protein arginine methyltransferase 2 as a coactivator for estrogen receptor alpha,” J. Biol. Chem. 277: 28624-30 (2002)). PRMT2-null mice are viable and grossly normal (Yoshimoto, T et al., “The arginine methyltransferase PRMT2 binds RB and regulates E2F function,” Exptl Cell Res. 312: 2040-53 (2006)).

The E2F family of transcription factors plays a crucial role in the control of cell cycle and action of tumor suppressor proteins and is also a target of the transforming proteins of small DNA tumor viruses. (Yoshimoto, T et al., “The arginine methyltransferase PRMT2 binds RB and regulates E2F function,” Exptl Cell Res. 312: 2040-53 (2006)). E2F proteins contain several evolutionally conserved domains found in most members of the family, including a DNA binding domain, a dimerization domain which determines interaction with the differentiation regulated transcription factor proteins (DP), a transactivation domain enriched in acidic amino acids, and a tumor suppressor protein association domain which is embedded within the transactivation domain. Id. E2F1, E2F2 and E2F3 have an additional cyclin binding domain. Id. E2F1 binds preferentially to retinoblastoma protein pRB in a cell-cycle dependent manner, and the retinoblastoma gene product (RB) is a regulator of E2F activity. Id. RB recruits a number of proteins, including HDACs, SWI/SNF complex, lysine methyl transferase (SUV39H1) and DNA methyltransferase (DNMT1), all of which negatively regulate E2F activity with RB. Id. PRMT2 directly binds and interacts with RB through its S-adenosylmethionine (AdoMet) binding domain, in contrast to other PRMT proteins, including PRMT1, PRMT3 and PRMT4. Id. In reporter assays, PRMT2 repressed E2F1 transcriptional activity in an RB-dependent manner. PRMT2 formed a ternary complex with E2F1 in the presence of RB. Id.

Experiments in which the PRMT2 gene was ablated in mice by gene targeting showed that, compared with PRMT2(+/+) mouse embryonic fibroblasts (MEFs), PRMT2(−/−) MEFs demonstrated increased E2F activity and early S phase entry following release of serum starvation. Id. Vascular injury to PRMT2(−/−) arteries results in a hyperplastic response, consistent with increased G1-S phase progression. Id.

PRMT3

PRMT3 harbors a zinc-finger domain at its N-terminus, which is its substrate recognition module (Bedford, M T, “Arginine methylation at a glance,” J. Cell Sci. 120: 4243-46 (2007), citing Tang, J, et al, “PRMT 3, a type 1 protein arginine N-methyltransferase that differs from PRMT1 in its oligomerization, subcellular localization, substrate specificity, and regulation, “J. Biol Chem. 273: 16935-45 (1998)). The 40S ribosomal protein S2 (rpS2) is a zinc-finger-dependent substrate of mammalian PRMT3 (Id., citing Swiercz, R et al., “Ribosomal protein rpS2 is hypomethylated in PRMT3-deficient mice,” J. Biol. Chem. 282: 16917-23 (2005)). In fission yeast the PRMT3-rpS2 substrate-enzyme pair exists (Bachand, F and Silver, P A, “PRMT3 is a ribosomal protein arginine methyltransferase that affects the cellular levels of ribosomal subunits,” EMBO J 23: 2641-50 (2004)), and the disruption of the prmt3 gene in this organism results in an imbalance in the 40S:60S free subunit ratio. Mouse PRMT3 are small, but survive after birth and attain a normal size in adulthood. The ribosome protein rpS2 is hypomethylated in the absence of PRMT3, which demonstrates that it is an in vivo PRMT3 substrate (Swiercz, R et al, “Ribosomal protein rpS2 is hypomethylated in PRMT3-deficient mice,” J. Biol. Chem. 282: 16917-23 (2007)).

PRMT4

The recruitment of PRMT 4, also known as CARM1, to promoters results in the methylation of histone H3 at Arg17 and of other coactivators including p300/CBP and AIB1 (Bedford, M T, “Arginine methylation at a glance,” J. Cell Sci. 120: 4243-46 (2007), citing Bedford, M T and Richard S, “Arginine methylation an emerging regulator of protein function,” Mol. Cell 18: 263-72 (2005)). CARM1-mediated methylation has a positive effect on transcription. CARM1 is both a steroid receptor coactivator and enhances transcription/translation rates in pathways responding to other transcription factors (Id., citing Bedford, M T and Richard S, “Arginine methylation an emerging regulator of protein function,” Mol. Cell 18: 263-72 (2005)). In addition, CARM1 methylates splicing factors and regulates the coupling of transcription and splicing (Id citing Cheng, D et al., “The arginine methyltransferase CARM1 regulates the coupling of transcription and mRNA processing,” Molec. Cell 25: 71-83 (2007)). CARM1-null mice die just after birth and are smaller than their wild-type littermates (Yadav, N et al., “Specific protein methylation defects and gene expression perturbations in coactivator-associated arginine methyltransferase 1-deficient mice,” Proc. Natl Acad. Sci. USA 100: 6464-68 (2003)). Cells from CARM1-null embryos have defective estrogen receptor and NF-κB pathways. CARM1 has also been implicated in the epigenetic programming of early embryos (Id., citing Torres-Padilla, M E et al., “Histone arginine methylation regulates pluripotency in the early mouse embryo,” Nature 445: 214-218 (2007)). As a coactivator for nuclear receptors, CARM1 is a likely candidate for overexpression in prostate and breast cancers; increased expression of CARM1 correlates with androgen independence in human prostate carcinoma (Id., citing Hong, H et al., “Aberrant expression of CARM1, transcriptional coactivator of androgen receptor in the development of prostate carcinoma and androgen-independent status,” Cancer 10: 83-89 (2004)) and CARM1 is overexpressed in breast tumors (Id., citing El Messaoudi, S et al., “co-activator-associated arginine methyltransferase 1 (CARM1) is a positive regulator of the cyclin E1 gene,” Proc. Natl Acad. Sci. USA 103: 13351-56 (2006)).

PRMT5

PRMT5 was cloned as a Janus kinase 2 (Jak2)-binding protein. (Bedford, M T, “Arginine methylation at a glance,” J. Cell Sci. 120: 4243-46 (2007)). Janus kinase 2, a protein tyrosine kinase, is part of the JAK/STAT signaling pathway, which, in mammals, is the principal signaling mechanism for a wide array of cytokines and growth factors. Rawlings, J S, et al, The JAK/STAT signaling pathway,” J. Cell Sci, 117: 1281-83 (2004). For example, the Janus kinase is required for signaling between the interferon receptor and STATs. (Pollack et al, “The Human Homologue of the Yeast Proteins Skb1 and Hsl7p Interacts with Jak Kinases and Contains Protein arginine methyltransferase Activity,” J. Biol. Chem. 274: 31531-42 (1999)).

The cellular localization of PRMT5 differs between nontransformed and transformed cells. In most primary and immortalized cells, PRMT5 is primarily located in the cytosol with a small amount in the nucleus; this distribution is reversed in transformed cells. Karkhanis, V et al, “Versatility of PRMT5-induced methylation in growth control and development,” Trends Biochem. Sci. 36(12): 633-41 (20110.

In the cytoplasm, PRMT5 is found in the ‘methylosome’, where it is involved in the methylation of Sm proteins. It has thus been implicated in snRNP biogenesis (Bedford, M T, “Arginine methylation at a glance,” J. Cell Sci. 120: 4243-46 (2007). citing Friesen, W J et al., “the methylosome, a 20S complex containing JBP1 and pICln, produces dimethylarginine-modified Sm proteins,” Mol. Cell. Biol. 21: 8289-8300 (2001)).

MEP50 is a component of the methylosome complex involved in the methylation and assembly of spliceosomal snRNP's Sm proteins. (Friesen, W J et al, “A novel WD repeat protein component of the methylosome binds Sm proteins,” J. Biol. Chem. 277: 8243-47 (2002)). The PRMT5-MEP50 complex can methylate members of the Janus kinase (JAK) family. Oncogenic JAK2 kinases promote myeloproliferative disease and erythroid differentiation, at least in part, by inactivating PRMT5, which may impinge on EGFR signaling. Karkhanis, V. et al, “Versatility of PRMT5-induced methylation in growth control and development,” Trends Biochem. Sci. 36(12): 633-41 (2011)).

Nuclear PRMT5 associates with regulators of transcriptional elongation SPT4 and SPT5 (Bedford, M T, “Arginine methylation at a glance,” J. Cell Sci. 120: 4243-46 (2007), citing Kwak, Y T et al., “Methylation of SPT5 regulates its interaction with RNA polymerase II and transcriptional elongation properties,” Mol. Cell. 11: 155-66 (2003)). Nuclear PRMT5 also forms complexes with the hSWI/SNF chromatin-remodeling proteins BRG and BRM, where it is responsible for methylating Arg8 on histone H3 (Id., citing Pal, S et al., “ ”Human SW1/SNF-associated PRMT5 methylates histone H3 arginine 8 and negatively regulates expression of ST7 and NM23 tumor suppressor genes,” Mol. Cell Biol. 24: 9630-45 (2004)).

PRMT5-induced epigenetic and post-translational changes have a significant impact on cell growth and proliferation. Karkhanis, V. et al, “Versatility of PRMT5-induced methylation in growth control and development,” Trends Biochem. Sci. 36(12): 633-41 (2011). Through multiple interactions with specific transcription factors and chromatin-modifying enzymes, PRMT5 is capable of controlling critical cellular pathways. For example, PRMT5 associates with specific ATP-dependent chromatin remodelers, corepressors as well as co-activators, and controls expression of key target genes involved in growth control, metastasis, differentiation, and development. Id. PRMT5 alone, or in combination with MEP50 interacts with a variety of transcriptional regulators to epigenetically modify specific histone arginine residues, including H3R8 and H4R3, and to induce gene silencing; PRMT5-induced H4R3 methylation orchestrates recruitment of repressor DNA methyltransferase DNMT3A. PRMT5 also promotes transcriptional silencing by post-translationally modifying chromatin-binding proteins, such as MBD2, p53, and CBP, and altering their biochemical properties. Id. PRMT5 knockdown resulted in decreased p53 stability and target gene expression, and ChIP assays indicated that decreased expression of CDKN1A, which encodes p21, was due to reduced p53 recruitment to its promoter. Id.

There is evidence that PRMT5 also plays a role in cell adhesion. Id. The SNAIL transcription factor, which is involved in the regulation of embryonic development and metastasis, regulates epithelial to mesenchymal transition by altering cell adhesion through transcriptional repression of E-cadherin (CDH1). Id. Several transcriptional co-repressors interact with SNAIL including AJUBA, which can bridge multiple interactions with several chromatin-modifying enzymes. Id. AJUBA forms a complex with PRMT5 and recruits it to SNAIL-repressed CDHI. Id. Knockdown of either AJUBA or PRMT5 abrogated their association with the CDH1 promoter and resulted in transcriptional derepression of CDH1.Id.

Association of PRMT5 with various binding partners appears to influence its substrate specificity. A yeast two-hybrid assay identified a nuclear protein dubbed cooperator of PRMT5 (COPR5) as a PRMT5 interacting partner. Id. COPR5-associated PRMT5 preferentially methylated histone H4R3 in vitro; however when PRMT5 is purified from cells in which COPR5 is knocked down, methylation of histone H3 is more prevalent, suggesting that the COPR5-PRMT-5 complex methylates H4 more efficiently than H3. Id. In vivo, both COPR5 and PRMT5 are enriched at the cyclin E (CCNE) promoter where the PRMT5-induced epigenetic mark H4R3 is hypermethylated; however COPR knockdown leads to transcriptional derepression of CCNE. Id. These experiments have been interpreted as showing that COPR5 is required for PRMT5 recruitment to the CCNE promoter, and indicate that COPR5 directs PRMT5 methyltransferase activity toward H4R3. Id.

PRMT6

PRMT6 is restricted to the nucleus and has the ability to methylate itself (Bedford, M T, “Arginine methylation at a glance,” J. Cell Sci. 120: 4243-46 (2007), citing Frankel, A. et al., “The novel human protein arginine N-methyltransferase PRMT6 is a nuclear enzyme displaying unique substrate specific,” J. Biol. Chem. 277: 3537-43 (2002)). Like PRMT1, PRMT6 methylates a GAR motif. Id. However, it methylates histones H3 and H4 in vitro, whereas PRMT1 only methylates histone H4 (Id., citing Lee, Y H et al., “Techniques in protein methylation,” Meths. Mol. Biol. 284: 195-208 (2004)). DNA polymerase β [Pol β] was found to form a complex with PRMT6. Methylation of Pol β by PRMT6 strongly stimulates DNA polymerase activity (Id., citing El-Andaloussi, N. et al, “Arginine methylation regulates DNA polymerase Beta,” Mol. Cell 22: 51-62 (2006)). PRMT6 is believed to play a role regulating DNA base excision repair. PRMT6 has also been shown to methylate a number of HIV proteins (Id citing Invernizzi, C F et al., “Arginine methylation of the HIV-1 nucleocapsid protein results in its diminished function,” AIDS 21: 795-805 (2007)). PRMT6 has recently been shown to methylate H3R2 (Id., citing Guccione, E et al., “Methylation of histone H3R2 by PRMT6 and H3K4 by an MLL complex are mutually exclusive,” Nature 449: 933-37 (2007)).

PRMT7

PRMT7 is one of two PRMTs that harbor two putative AdoMet-binding motifs (Id., citing Miranda, T B et al., “PRMT7 is a member of the protein arginine methyltransferase family with a distinct substrate specificity,” J. Biol. Chem. 279: 22902-907 (2004)). It has a strong propensity to catalyze the formation of monomethylarginine (ω-N^{G}-monomethylarginine, MMA) but not dimethylarginine (DMA) on a fibrillarin-derived peptide substrate (Id., citing Miranda, T B et al., “PRMT7 is a member of the protein arginine methyltransferase family with a distinct substrate specificity,” J. Biol. Chem. 279: 22902-907 (2004)). While Miranda et al. classified PRMT7 as a type III enzyme. Lee et al. using a different peptide substrate, showed that PRMT7 catalyzes the formation of symmetric dimethylarginine (ω-N^{G},N′^{G}-symmetric dimethylarginine; sDMA), consequently classifying it as a type II enzyme (Id., citing Lee, J H et al., “PRMT7, a new protein arginine methyltransferase that synthesizes symmetric dimethylarginine,” J. Biol. Chem. 280: 3656-64 (2005)), suggesting that distinct substrates may be methylated in different fashions by this enzyme. A study that focused on identifying loci that impart susceptibility to drug-induced nephropathy implicated PRMT7 as a candidate (Id., citing Zheng, Z et al., “A Mendelian locus on chromosome 16 determines susceptibility to doxorubicin nephropathy in the mouse,” Proc. Natl. Acad. Sci. USA 102: 2502-2507 (2005)). Also, PRMT7 plays a role in male germline imprinted gene methylation through its interaction with CTCFL (a protein that associates with the imprinting control region) and subsequent methylation of histone 4 Arg3 (Id citing Jelinic, P et al., “The testis-specific factor CTCFL cooperates with the protein methyl transferase PRMT7 in H19 imprinting control region methylation,” PLoS Biol. 4: e355 (2006)).

PRMT8

The N-terminal of PRMT8 harbors a myristoylation motif that facilitates its association with the plasma membrane. (Bedford, M T, “Arginine methylation at a glance,” J. Cell Sci. 120: 4243-46 (2007)). It is largely restricted to the brain.

PRMT9 (4q31)

In common with PRMT7, PRMT9 harbors two putative AdoMet-binding motifs. In addition, at its N-terminus PRMT9 has a TPR repeat, which may be a protein-protein interaction module (Id., citing Bedford, M T, “The family of protein arginine methyltranferases,” in The Enzymes, S. G. Clarke and F. Tamanoi, Ed., pp. 31-50, Amsterdam: Academic Press).

FBXO11, also referred to as PRMT9 (2p16.3), was identified as a potential PRMT because it has regions that display weak sequence similarity to the I, post-I, II and III amino acid sequence motifs (Id., citing Cook, J R et al., “FBXO11/PRMT9, a new protein arginine methyltransferase, symmetrically dimethylates arginine residues,” Biochem. Biophys. Res. Communic. 342: 472-81 (2006)). Unlike other PRMTs, it does not harbor a THW loop. Although FLAG-tagged hFBXO11 has been reported to have type II activity (Id citing Cook, J R et al., “FBX011/PRMT9, a new protein arginine methyltransferase, symmetrically dimethylates arginine residues,” Biochem. Biophys. Res. Communic. 342: 472-81 (2006)), HA-tagged hFBXO11 and its C. elegans ortholog (DRE-1) have been reported not to have PRMT activity (Fielenbach, N et al., DRE-1: an evolutionarily conserved F box protein that regulates C. elegans developmental age,” Dev. Cell 12: 443-55 (2007)).

PRMT Substrates

The wide range of arginine methylated substrates suggests that this eukaryotic modification may parallel phosphorylation in its level of complexity. (McBride, A E, “State of the Arg: Protein Methylation at Arginine comes of age,” Cell 106: 5-8 (2001)).

Proteins that harbor GAR motifs are often targets for PRMTs; CARM1, which cannot methylate a GAR motif, is an exception. PRMT1 has a high affinity for GAR regions, and the majority of identified methylated arginines are located within such domains. (van Dijk, T B, et al, “Friend of PRMT1, a novel chromatin target of protein arginine methyltransferases,” Mol. Cell. Biol. 30(1): 260-72 (2010)). GAR regions are a common feature of many RNA-binding proteins, (RBPs) including the heterogeneous ribonucleoproteins (hnRNPs), which play roles in mRNA processing and transport and contain up to 65% of total nuclear dimethylarginine (DMA). Id. It remains to be determined whether methylation has a profound effect on protein-RNA interactions.

Both CARM1 and PRMT5 can also methylate proline-, glycine-, methionine-, arginine-rich patches (PGM motifs) (Bedford, M T, “Arginine methylation at a glance,” J. Cell Sci. 120: 4243-46 (2007), citing Cheng, D et al., “The arginine methyltransferase CARM1 regulates the coupling of transcription and mRNA processing,” Mol. Cell 25: 71-83 (2007)), which are found in a number of splicing factors (Id., citing Bedford M T et al., “WW domain-mediated interactions reveal a spliceosome-associated protein that binds a third class of proline-rich motif: the proline glycine and methionine-rich motif,” Proc. Natl Acad. Sci. USA 95: 10602-607 (1998).

Another mechanism of substrate recognition is regulated via controlled recruitment. (van Dijk, T B, et al, “Friend of PRMT1, a novel chromatin target of protein arginine methyltransferases,” Mol. Cell. Biol. 30(1): 260-72 (2010)). For example, PRMTs are recruited to promoters and other regulatory elements to control gene expression by the methylation of histones and components of the transcription machinery. Id. The recruitment of PRMT1 by nuclear hormone receptors and the transcription factors p53, YY1/Drbp76 and upstream stimulatory factor 1 (Usf1) results in the local methylation of histone H4 at R3, which is critical for subsequent histone acetylation and further activation events. Id.

Protein arginine methylation has been implicated in a number of cellular processes, including transduction of intracellular signaling (Zakrzewicz, D et al, “Protein Arginine Methyltransferases (PRMTs): Promising Targets for the treatment of pulmonary disorders,” Intl J. Mol. Sci. 13: 12383-400 (2012), citing Blanchet, F. et al, “Protein arginine methylation in lymphocyte signaling,” Curr. Opin. Immunol. 18: 321-28 (2006)), DNA repair, Zakrzewicz, D et al, “Protein Arginine Methyltransferases (PRMTs): Promising Targets for the treatment of pulmonary disorders,” Intl J. Mol. Sci. 13: 12383-400 (2012), citing Lake, A N, and Bedford, M T, “Protein methylation and DNA repair,” Mutat. Res. 618: 91-101 (2007); El-Andaloussi, N. et al, “Methylation of DNA polymerase beta by protein arginine methyltransferase 1 regulates its binding to proliferating cell nuclear antigen,” FASEB J. 21: 26-34 (2007); El-Andaloussi, N. et al, “Arginine methylation regulates DNA polymerase beta,” Mol. Cell 22: 51-62 (2006)), RNA processing (Zakrzewicz, D et al, “Protein Arginine Methyltransferases (PRMTs): Promising Targets for the treatment of pulmonary disorders,” Intl J. Mol. Sci. 13: 12383-400 (2012), citing Cheng, D. et al, “The arginine methyltransferase CARM1 regulates the coupling of transcription and mRNA processing,” Mol. Cell 25: 71-83 (2007); Boisvert, F M et al, “Symmetrical dimethylarginine methylation is required for the localization of SMN in Cajal bodies and pre-mRNA splicing,” J. Cell Biol. 159: 957-969 (2002), protein-protein interaction, and regulation of gene expression (Zakrzewicz, D et al, “Protein Arginine Methyltransferases (PRMTs): Promising Targets for the treatment of pulmonary disorders,” Intl J. Mol. Sci. 13: 12383-400 (2012), citing Infantino, S. et al, “Arginine methylation of the B cell antigen receptor promotes differentiation,” J. Exptl Med. 207: 711-19 (2010); Yu, Z et al, “A mouse PRMT1 null allele defines an essential role for arginine methylation in genome maintenance and cell proliferation,” Mol. Cell Biol. 29: 2982-96 (2009).

Although methylation does not change the overall charge on an arginine residue, addition of methyl groups increases steric hindrance and hydrophobicity and decreases hydrogen bonding capacity by removing amino hydrogens that might be involved in hydrogen bonds. (Id.; van Dijk, T B, et al, “Friend of PRMT1, a novel chromatin target of protein arginine methyltransferases,” Mol. Cell. Biol. 30(1): 260-72 (2010)). Further, methylation protects the reactive guanidine groups of arginine residues against inappropriate modification by dicarbonyl reagents. (van Dijk, T B, et al, “Friend of PRMT1, a novel chromatin target of protein arginine methyltransferases,” Mol. Cell. Biol. 30(1): 260-72 (2010), citing Fackelmayer, F O, “Protein arginine methyltransferases: guardians of the Arg?”, Trends Biochem. Sci. 30: 666-71 (2005)).

Arginine Methylation Regulates Protein-Protein Interactions

Arginine methylation facilitates the interaction of GAR and PGM motifs with Tudor domains. (Bedford, M T, “Arginine methylation at a glance,” J. Cell Sci. 120: 4243-46 (2007)). The TUDOR domain was originally identified as a region of 50 amino acids found in the Drosophila TUDOR protein (a posterior group gene). The TUDOR domain has been found within a number of proteins involved in RNA binding.

Methylation appears to affect the binding of nucleic acid binding proteins to protein partners in a nucleic acid-independent manner. (McBride, A. E. and Silver, P. A., “State of the Arg: Protein Methylation at Arginine Comes of Age,” Cell 106: 5-8 (2001)).

For example, the symmetric dimethylation of CARM1 substrate SmB by PRMT5 is required for its interaction with the Tudor domains of Survival of Motor Neuron (SMN), human splicing factor SPF30 and TDRD3, a transcriptional coactivator that promotes transcription by binding methylarginine marks on histone tails ((Bedford, M T, “Arginine methylation at a glance,” J. Cell Sci. 120: 4243-46 (2007), citing Yang, Y. et al, “TDRD3 is an effector molecule for arginine methylated histone marks,” Molecular Cell 40(6): 1016-23 (2010); Cote, J and Richard, S., “Tudor domains bind symmetrical dimethylated arginines,” J. Biol. Chem. 280: 28476-28483 (2005)). The three-dimensional TUDOR domain structure of human SMN forms a strongly bent, anti-parallel β-sheet consisting of five β-strands and a barrel-like fold. The asymmetric dimethylation of CA150 by CARM1 also provides a docking site for the Tudor domain of SMN (Id. Citing Cheng, D. et al, “The arginine methyltransferase CARM1 regulates the coupling of transcription and mRNA processing,” Mol. Cell 25: 71-83 (2007)). Thus, motifs harboring either aDMA or sDMA residues bind a subset of Tudor-domain-containing proteins. The methyl-binding pocket may be a conserved aromatic ‘cage’ in Tudor domains (Id. Citing Sprangers, R. et al., “High resolution X ray and NMR structures of the SMN Tudor domain: conformational variation in the binding site for symmetrically dimethylated arginine residues,” J. Mol. Biol. 327: 507-20 (2003)).

The 294-residue SMN protein is part of a multimeric complex that includes the spliceosomal Sm core proteins 5-8. Selenko, P. et al, “SMN Tudor domain structure and its interaction with the Sm proteins,” Nature Structural Biol. 8(1): 27-31 (2001)). The seven human Sm core proteins are Sm B, D1-3, E, F, and G. Most of the cellular SMN protein is localized in the cytoplasm, where it is crucial for the assembly of spliceosomal uridine-rich small nuclear ribonucleoprotein (U snRNP) complexes (Id.). It has been shown that the interaction of SMN with Sm proteins is essential for this process, during which hetero-oligomeric Sm D1-D2, E-F-G and D3-B complexes are bound to the U snRNAs. Id. SMN contains a central, highly conserved Tudor domain that is required for U snRNP assembly and facilitates Sm protein binding. Id.

Arginine methylation can also act as a negative regulator of protein-protein interactions. ((Bedford, M T, “Arginine methylation at a glance,” J. Cell Sci. 120: 4243-46 (2007)).

For example, Sam68, a mitotic substrate for the Src kinase, which is thought to act as an adaptor protein in signaling pathways, binds to both WW domain and SH3 domain-containing proteins through proline-rich regions. (McBride, A E, and Silver, P A, “State of the Arg: Protein methylation at arginine comes of age,” Cell 106: 5-8 (2001)). The methylation of Sam68 at arginine residues adjacent to a proline-rich motif can block binding to SH3, but not WW, domains (Id., citing Bedford, M T et al., “Arginine methylation inhibits the binding of proline-rich ligands to Src homology 3, but not WW, domains,” J. Biol. Chem. 275: 16030-36 (2000)), suggesting that methylation may be involved in switching the function of Sam68 by altering specific protein-protein contacts.

Another example is the CARM1-mediated modification of the glucocorticoid receptor-interacting protein 1 (GRIP1)-binding domain of p300 (a transcriptional coactivator that is a ubiquitous nuclear phosphoprotein and transcriptional cofactor with intrinsic acetyltransferase activity; see Ghosh, A K and Varga, J., “The transcriptional coactivator and acetyltransferase p300 in fibroblast biology and fibrosis,” J. Cell Physiol. 213(3): 663-71 (2007)) (Bedford, M T, “Arginine methylation at a glance,” J. Cell Sci. 120: 4243-46 (2007), citing, Lee, Y H et al, “Regulation of coactivator complex assembly and function by protein arginine methylation and demethylimination,” Proc. Natl Acad. Sci. USA 102: 3611-16 (2005).

Histone H3 methylation at Lys4 provides a docking site for the double chromodomains of CHD1 (chromo-helicase/ATPase DNA binding protein 1). The histone H3 Arg2 site is reported to be methylated by CARM1, and this methylation together with Lys4 methylation decreases the binding affinity fourfold relative to histone H3 Lys4 methylation alone (Bedford, M T, “Arginine methylation at a glance,” J. Cell Sci. 120: 4243-46 (2007), citing Flanagan, J F et al., “Double chromodomains cooperate to recognize the methylated histone H3 tail,” Nature 438: 1181-85 (2005)).

Regulation of Arginine Methylation

Post-translational modification of PRMTs has been shown to regulate arginine methyltransferase activity. Yang, Y and Bedford, M T, “Protein arginine methyltransferases and cancer,” Nature Rev. Cancer 13: 37-50 (2013). For example, modulator protein interaction regulates arginine methylation (for example, the PRMT5-methylosome protein 50 (MEP50) interaction). Id. Adaptor proteins bridge PRMTs and specific substrates for methylation; for example, mutually exclusive binding of RIO kinase 1 (RIOK1) and ICLN with PRMT5 facilitates distinct substrate methylation. Id. The existence of posttranslational modifications on the substrate facilitates or prevents adjacent arginine methylation by PRMTs. Id. In general, phosphorylation blocks arginine methylation, and acetylation stimulates arginine methylation (although there are exceptions). Id. A number of the PRMTs shuttle between the nucleus and the cytoplasm, and PRMT8 can be attached to the plasma membrane through a myristoylation event. Id. In addition, PRMT regulation can be achieved through their restriction to subcellular compartments. Id. Further, PRMTs are targeted for mRNA cleavage and translational repression by microRNAs.

PRMT-binding proteins can regulate the activity of PRMTs. They can inhibit, activate, or change the substrate specificity of PRMTs.

In mammalian cells, PRMT5 is tightly bound by MEP50, and this interaction is required for PRMT5 activity. Id., citing Friesen, W J et al, “A novel WD repeat protein component of the methylosome binds Sm proteins,” J. Biol. Chem. 277: 8243-47 (2002)). Tyrosine phosphorylation of PRMT5 can block MEP50 binding and can attenuate PRMT 5 activity. Id., citing Liu, F. et al, “JAK2V617F-mediated phosphorylation of PRMT5 downregulates its methyltransferase activity and promotes myeloproliferation,” Cancer Cell 19: 283-94 (2011)). Phosphorylation of MEP50 by the cyclin D1-cyclin-dependent kinase 4 (CDK4) complex further enhances MEP50-PRMT5 methyltransferase activity and triggers neoplastic growth in vitro. Id, citing (Aggarwal, P. et al, “Nuclear cyclin D1/CDK kinase regulates CUL4 expression and triggers neoplastic growth via activation of the PRMT5 methyltransferase,” Cancer Cell 18: 329-40 (2010)).

Although MEP50 is the primary regulator of PRMT5 activity, other binding proteins can function as modulators or adaptors of this complex. These include the SWI/SNF chromatin remodeling complex, which enhances MEP50-PRMT5 methyltranferase activity towards histone substrates (Id, citing Pal, S et al., “ ”Human SW1/SNF-associated PRMT5 methylates histone H3 arginine 8 and negatively regulates expression of ST7 and NM23 tumor suppressor genes,” Mol. Cell Biol. 24: 9630-45 (2004)); the exon junction complex component and RNA-binding protein Y14 (also known as RBM8A), which increases the activity of MEP50-PRMT5 towards Smith antigen (Sm) proteins of the small nuclear ribonucleoprotein core (Id., citing Chuang, T W et al, “The exon junction complex component Y14 modulates the activity of the methylosome in biogenesis of spliceosomal small nuclear ribonucleoproteins,” J. Biol. Chem., 286: 8722-28 (2011)); RIO kinase 1 (RIOK1) and chloride conductance regulatory protein ICLN, which are adaptors that bind MEP50-PRMT5 in a mutually exclusive manner and regulate substrate specificity (Id., citing Guderian, G. et al, “RioK1, a new interactor of protein arginine methyltransferase 5 (PRMT5) competes with PICln for binding and modulates PRMT5 complex composition and substrate specificity,” J. Biol. Chem. 286: 1976-86 (2011))); the histone-binding protein cooperator of PRMT5 (COPR5), which guides MEP50-PRMT5 to methylate histone H4R3 rather than histone H3R8 (Id., citing Lacroix, M. et al, “The histone-binding protein COPR5 is required for nuclear functions of the protein arginine methyltransferase PRMT5,” EMBO Rep. 9: 452-58 (2008)); and head shock protein 90 (HSP90), which stabilizes PRMT4 protein levels.

PRMT1 activity is negatively regulated by the orphan nuclear receptor TR3 (also known as NR4A1) (Id citing Lei, N Z et al, “a feedback regulatory loop between methyltransferase PRMT1 and orphan receptor TR3,” Nucleic Acid Res. 37: 832-48 (2009)). Similarly, the BTG1-binding chromatin assembly factor 1 (CAF1) complex inhibits PRMT1 enzyme activity (Id., citing Robin-Lespinasse, Y et al., “hCAF1, a new regulator of PRMT1-dependent arginine methylation,” J. Cell Sci. 120: 638-47 (2007)), although BTG1 alone stimulates PRMT1 activity towards selected substrates. (id citing Lin, W J et al., “The mammalian intermediate-early T1S21 protein and the leukemia-associated BTG1 protein interact with a protein-arginine N-methyltransferase,” J. Biol. Chem. 271: 15034-44 (1996)). PRMT1 activity also is reported to be increased by its ability to heterodimerize with PRMT2 (Id citing Pak, M L et al, “A protein arginine N-methyltransferase 1 (PRMT1) and 2 heteromeric interactions increases PRMT1 enzymatic activity,” Biochem 50: 8226-40 (2011).

Binding of the tumor suppressor DAL-1 (also known as 4.1B) to PRMT3 acts as an inhibitor of enzyme activity, both in vitro and in cell lines (Id., citing Singh, V et al., “DAL-1/4.1B tumor suppressor interacts with protein arginine N-methyltransferase 3 (PRMT3) and inhibits its ability to methylate substrates in vitro and in vivo,” Oncogene 23: 7761-77 (2004)).

CARM1 is found in a complex of at least 10 proteins called the nucleosomal methylation activator complex (NUMAC) (Id., citing Xu, W et al., “a methylation-mediator complex in hormone signaling,” Genes Dev. 18: 144-156 (2004)). NUMAC targets CARM1 to methylate nucleosomal histone H3. (Id).

The binding of CCCTC-binding factor like (CTCFL) to PRMT7 bridges PRMT7 and its histone substrates (Id citing Jelinic, P et al., “The testis-specific factor CTCFL cooperates with the protein methyl transferase PRMT7 in H19 imprinting control region methylation,” PLoS Biol. 4: e355 (2006)).

Arginine residues within proteins can be converted to citrulline by deimination. A major group of deiminated proteins are the core histones H2A, H3 and H4 (Id., citing Nakashima, K et al., “Nuclear localization of peptidylarginine diminase V and histone deamination in graulocytes,” J Biol Chem. 277: 49562-68 (2002)). The peptidyl arginine deiminases (PADs) can block methylation on an arginine residue by converting it to citrulline (Id., citing Cuthbert, G L et al., “Histone deamination antagonizes arginine methylation,” Cell 118: 543-53 (2004); Wang, Y et al., “Human PAD4 regulates histone arginine methylation levels via demethylimination,” Science 306: 279-83 (2004)). PADs catalyze the deimination of arginine, but not MMA or DMA, to citrulline (Id., citing Raijmakers, R. et al., “Methylation of arginine residues interferes with citrullination by peptidylarginine deiminases in vitro,” J. Mol. Biol. 367: 1118-29 (2007)). Thus, peptidyl arginine deiminases are not demethylases. However, these enzymes may carry out a preemptive strike on key sites of arginine methylation, thereby preventing subsequent methylation. Higashimoto, K et al, “Phosphorylation-mediated inactivation of coactivator-associated arginine methyltransferase I,” Proc. Natl Acad. Sci. USA 104: 12318-323 (2007)) showed that PRMTs themselves are regulated by posttranslational events. In this case, the phosphorylation of CARM1 results in a decrease in PRMT activity.

JMJD6, the first arginine demethylase identified (Bedford, M T, “Arginine methylation at a glance,” J. Cell Sci. 120: 4243-46 (2007), citing Chang, B. et al, “JMJD6 is a histone arginine demethylase,” Science 318: 444-47 (2007)) is a Jimonji-domain-containing protein. A Jimonji domain (JmJC) is a protein domain in the jumonji family of transcription factors. Proteins containing JmjC domains are predicted to be metalloenzymes that adopt the cupin fold and are candidates for enzymes that regulate chromatin remodelling (Clissold, P M, Ponting C P, “JmjC: cupin metalloenzyme-like domains in jumonji, hairless and phospholipase A2beta,” Trends in Biochem. Sci. 26(1): 7-9 (2001)). The cupin fold is a flattened beta-barrel structure containing two sheets of five antiparallel beta strands that form the walls of a zinc-binding cleft. Based on the crystal structure of JmjC domain containing protein FIH and JHDM3A/JMJD2A, the JmjC domain forms an enzymatically active pocket that coordinates Fe(III) and alphaKG. Three amino-acid residues within the JmjC domain bind to the Fe(II) cofactor and two additional residues bind to alphaKG (Klose, R J et al, JmjC-domain-containing proteins and histone demethylation,” Nature Reviews. Genetics: 7 (9): 715-27 (2006)).

JmjC domains have been identified in numerous eukaryotic proteins containing domains typical of transcription factors, such as PHD, C2H2, ARID/BRIGHT and zinc fingers (Clissold, P M, Ponting C P, “JmjC: cupin metalloenzyme-like domains in jumonji, hairless and phospholipase A2beta,” Trends in Biochem. Sci. 26(1): 7-9 (2001); Ellins, J M et al, “Structure of factor-inhibiting hypoxia-inducible factor (HIF) reveals mechanism of oxidative modification of HIF-1 alpha,” J. Biol. Chem. 278(3): 1802-06 (2003)). The JmjC has been shown to function in a histone demethylation mechanism that is conserved from yeast to human (Tsukada, Y. et al, “Histone demethylation by a family of JmjC domain-containing proteins,” Nature 439 (7078): 27599-816 (2006). JmjC domain proteins may be protein hydroxylases that catalyze a novel histone modification (Trewick, S C et al, “Methylation: lost in hydroxylation?”, EMBO Reports 6(4): 315-20 (2005)). The human JmjC protein named Tyw5p acts in the biosynthesis of a hypermodified nucleoside, hydroxy-wybutosine, in tRNA-Phe by catalyzing hydroxylation (Noma, A. et al, “Expanding role of the jumonji C domain as an RNA hydroxylase,” J. Biol. Chem. 285 (45): 34503-07 (2010)).

Methylation of arginine residues within proteins by PRMTs and the subsequent proteolysis of these arginine-methylated proteins by proteasome and autophagy pathways represent the major source of free intracellular methylarginine, since there is no evidence to date that free L-arginine can be methylated. (Zakrzewicz, D. et al, “Protein arginine methyltransferases (PRMTs): promising targets for the treatment of pulmonary disorders,” Intl J. Molec. Sci. 13: 12383-400 (2012))

Nuclear Receptors

The nuclear receptor superfamily describes a related but diverse array of transcription factors, which include nuclear hormone receptors (NHRs) and orphan nuclear receptors. J. M. Olefsky, “Nuclear Receptor Minireview Series,” J. Biol. Chem. 276: 36863-64 (2001). NHRs are receptors for which hormonal ligands have been identified, whereas orphan receptors are so named because their ligands are unknown, at least at the time the receptor is identified. Although lipid-soluble hormones (e.g., glucocorticoids, mineralocorticoids, the sex steroids (estrogen, progesterone, and androgen), thyroid hormones, and vitamin D3) can diffuse through the plasma membrane and interact directly with transcription factors in the cytoplasm or nucleus, a second major class of hormones (peptide and protein hormones) function by binding to specific cell-surface receptors, which then pass the signal that they have bound hormone to proteins within the cell by signal transduction. (H. Lodish, et al., Molecular Cell Biology, Ch. 10, “Regulation of Transcription Initiation,” W.H. Freeman and Co., New York (2000)).

Exemplary nuclear receptors are shown in the following table, Table 1:

| | | |
|---|---|---|
| Receptor | | Human |
| Nomenclature | Receptor Common Name | Gene Name |

Type 1A: Thyroid Hormone Receptors

| | | |
|---|---|---|
| NR1A1 | Thyroid hormone receptor-α | THRA |
| NR1A2 | Thyroid hormone receptor-β | THRB |

Type 1B: Retinoic Acid Receptors

| | | |
|---|---|---|
| NR1B1 | Retinoic acid receptor-α | RARA |
| | (RARα) | |
| NR1B2 | Retinoic acid receptor-β | RARB |
| | (RARβ) | |
| NR1B3 | Retinoic acid receptor-γ | RARG |
| | (RARγ) | |

Type 1C: Peroxisome Proliferator-Activated Receptors (PPAR)

| | | |
|---|---|---|
| NR1C1 | Peroxisome proliferator- | PPARA |
| | activated receptor-α (PPAR α) | |
| NR1C2 | Peroxisome proliferator- | PPARD |
| | activated receptor-β/δ (PPAR | |
| | β/δ) | |
| NR1C3 | Peroxisome proliferator- | PPARG |
| | activated receptor-γ (PPAR γ) | |

Type 1F: RAR-Related Orphan Receptors

| | | |
|---|---|---|
| NR1F1 | RAR-related orphan receptor-α | RORA |
| NR1F2 | RAR-related orphan receptor-β | RORB |
| NR1F3 | RAR-related orphan receptor-γ | RORC |

Type 1H: Liver X Receptor-Like Receptors

| | | |
|---|---|---|
| NR1H2 | Liver X receptor-β (LXRβ) | NR1H2 |
| NR1H3 | Liver X receptor -α (LXRα) | NR1H3 |

Farnesoid X Receptors

| | | |
|---|---|---|
| NR1H4 | Farnesoid X receptor(FXR) | NR1H4 |
| NR1H5 | Farnesoid X receptor-β(FXRβ) | NR1H5P |

Type 1I: Vitamin D Receptor-Like Receptors

| | | |
|---|---|---|
| NRI1 | Vitamin D receptor (VDR) | VDR |
| NRI2 | Pregnane X receptor (PXR) | NR1I2 |
| NRI3 | Constituitive androstane | NR1I3 |
| | receptor (CAR) | |

Type 2A: Hepatocyte Nuclear Factor-4 (HNF4) Receptors

| | | |
|---|---|---|
| NR2A1 | Hepatocyte nuclear factor-4-α | HNF4A |
| | (HNF-4-α) | |
| NR2A2 | Hepatocyte nuclear factor 4-γ | HNF4G |
| | (HNF4-γ) | |

Type 2B: Retinoid X receptors (RXR)

| | | |
|---|---|---|
| NR2B1 | Retinoid X receptor-α (RXRα) | RXRA |
| NR2B2 | Retinoid X receptor-β (RXRβ) | RXRB |
| NR2B3 | Retinoid X receptor-γ (RXRγ) | RXRC |

Type 0B: DAX-Like Receptors

| | | |
|---|---|---|
| NR0B1 | DAX1 | NR0B1 |
| NR0B2 | Small heterodimer partner, | NR0B2 |
| | SHP | |

(Source: the medicalbiochemistrypage.org/nuclear.php, last modified Jan. 22, 2015, visited Mar. 5, 2015)

All nuclear receptors have common structural features, which include a central DNA binding domain (DBD) responsible for targeting the receptor to highly specific DNA sequences comprising a response element that binds the nuclear receptor. (J. M. Olefsky, “Nuclear Receptor Minireview Series,” J. Biol. Chem. 276: 36863-64 (2001)). The ligand binding domain (LBD) is contained in the C-terminal half of the receptor and recognizes specific hormonal and nonhormonal ligands directing specificity to the biologic response. Id. These receptors contain variable N-terminal and C-terminal domains, as well as a variable length hinge region between the DBD and LBD. Id. Nuclear receptors can exist as homo- or heterodimers with each partner binding to specific RE sequences that exist as half-sites separated by variable length nucleotide spacers between direct or inverted half-site repeats. Id. Class 1 receptors include the known steroid hormone receptors, which function as homodimers binding to half-site RE inverted repeats. Class 2 receptors exist as heterodimers with RXR receptor partners and function in a ligand-dependent manner. Orphan receptors function as homodimers binding to direct RE repeats (Class 3) or monomers binding to single site REs (Class 4).

Activation of transcription by nuclear receptors involves the recruitment of coactivator proteins. Agonist ligands promote recruitment of coactivator proteins to the receptor by stabilization of the C-terminal AF-2 helix of the ligand binding domain in a conformation that forms a “charge clamp”. (see Nolte et al, Nature (1998) and Shiau, A. K. et al., Cell (1998), Vol. 95, pp 927-937). Structural studies suggest that many nuclear receptors share a similar general mechanism of activation, where binding of ligand stabilizes the AF2 helix, thereby stabilizing the charge clamp and allowing coactivators to bind. X-ray structures of the estrogen receptor, progesterone receptor, thyroid receptor, retinoic acid receptor and vitamin D receptor show that, in these cases, the ligand generally makes lipophilic contacts with the AF2 helix.

Repression of gene transcription by nuclear receptors is mediated by interactions with co-repressor proteins such as SMRT and N-CoR1, which in turn recruit histone deacetylases to the chromatin. (Xu, H E et al, “Structural basis for antagonist mediated recruitment of nuclear co-repressors by PPARα,” Nature 415: 813-17 (2002)).

Heterodimeric nuclear receptors are located exclusively in the nucleus. (H. Lodish, et al., Molecular Cell Biology, Ch. 10, “Regulation of Transcription Initiation,” W.H. Freeman and Co., New York (2000)). Homodimeric receptors are found both in the cytoplasm and nucleus, and their activity is regulated by controlling their transport from the cytoplasm to the nucleus. Id.

When heterodimeric nuclear receptors (e.g., retinoid X-receptor-Vitamin D receptor (RXR-VDR), retinoid X receptor-thyroid hormone receptor (RXR-TR), and retinoid X receptor-retinoic acid receptor (RXR-RAR)) are bound to their cognate sites in DNA, they act as repressors or activators of transcription depending on whether hormone occupies the ligand-binding site. Id. In the absence of hormone, they direct histone deacetylation at nearby nucleosomes. Id. In the presence of hormone, the ligand-binding domain undergoes a dramatic conformational change. Id. In the ligand-bound conformation, these nuclear receptors can direct hyperacetylation of histones in nearby nucleosomes, thereby reversing the repressing effects of the free ligand-binding domain. Id.

The translocation of the homodimeric glucocorticoid receptor (GR) is mediated by the GR hormone binding domain. In the absence of hormone, the glucocorticoid receptor is anchored in the cytoplasm as a large protein aggregate complexed with inhibitor proteins, including Hsp90. Id. Since the receptor cannot interact with target genes in this situation, no transcriptional activation occurs. Binding of hormone releases the glucocorticoid receptor from its cytoplasmic anchor, allowing it to enter the nucleus where it can bind to response elements associated with target genes. Once the receptor with bound hormone interacts with a response element, it activates transcription by directing histone hyperacetylation and facilitating cooperative assembly of an initiation complex.

Peroxisome Proliferator-Activated Receptors (PPARs)

The peroxisome proliferator-activated receptors (PPARs) are a group of nuclear receptor proteins that function as transcription factors regulating the expression of genes. All PPARs are known to heterodimerize with the retinoid X receptor (RXR) and bind to specific regions on the DNA of target genes called peroxisome proliferator hormone response elements (PPREs). PPARs play essential roles in the regulation of cellular differentiation, development, and metabolism (carbohydrate, lipid, protein), and tumorigenesis of higher organisms. The family comprises three members, PPAR-α, PPAR-γ, and PPAR-δ (also known as PPAR-β). PPAR-α is expressed in liver, kidney, heart, muscle, adipose tissue, as well as other tissues. PPAR-δ is expressed in many tissues but markedly in brain, adipose tissue, and skin. PPAR-γ comprises three alternatively-spliced forms, each with a different expression pattern. PPAR-γ1 is expressed in virtually all tissues, including heart, muscle, colon, kidney, pancreas, and spleen. PPAR-γ2 is expressed mainly in adipose tissue. PPAR-γ3 is expressed in macrophages, large intestine, and white adipose tissue. Endogenous ligands for the PPARs include free fatty acids and eicosanoids. PPAR-γ is activated by PGJ2 (a prostaglandin), whereas PPAR-α is activated by leukotriene B4.

Peroxisome proliferator-activated receptors (PPARs, including PPARα, PPARγ, and PPARδ (also referred to as PPARβ, NUC1, and FAAR) exert diverse effects on fat and carbohydrate metabolism and are major targets for therapeutic agents in metabolic diseases.

PPARs possess the canonical domain structure of other NHR superfamily members, including a poorly characterized N-terminal region that contains a potential trans-activation function known as AF-1, followed by a DNA binding domain that includes two zinc fingers. Rosen, E D and Spiegelman, B M, “PPAR γ: a nuclear regulator of metabolism, differentiation, and cell growth,” J. Biol. Chem. 276: 37731-34 (2001). At the carboxyl terminus is a dimerization and ligand binding domain that molecular modeling reveals to be a large hydrophobic pocket and which contains a key, ligand-dependent trans-activation function called AF-2. Id. PPARs bind to cognate DNA elements called PPAR response elements (PPREs) in the 5′-flanking region of target genes. Id. Like many other NHRs, they bind DNA as obligate heterodimers by partnering with one of the retinoid X receptors (RXRs). Id. Known PPREs are direct repeats of an AGGNCA half-site separated by a 1-base pair spacer. Id. A short sequence located immediately upstream of the first half-site confers polarity on the PPRE, with the PPAR moiety binding 5′ to the RXR half of the heterodimer. Id. Many cell types express more than one PPAR isoform, which suggests that isoform-specific targets are regulated through a combination of subtle cis sequence differences flanking the core response element, the presence of specific or selective coactivator proteins, and regulation of endogenous ligands. Id.

PPARs, like other NHRs, form protein-protein interactions with a variety of nuclear proteins known as coactivators and corepressors, which mediate contact between the PPAR-RXR heterodimer, chromatin, and the basal transcriptional machinery and which promote activation and repression of gene expression, respectively. Id. Coactivator proteins, which include members of the p160/CBP/p300 and DRIP/TRAP families, are general coactivators for NHRs and indeed many non-NHR transcription factors. Id. There are no known receptor-specific coactivators or corepressors, although selectivity for one or another NHR has been illustrated in certain cases. Id. Coactivator proteins either possess or recruit histone acetyltransferase (HAT) activity to the transcription start site. Id. Acetylation of histone proteins is believed to relieve the tightly packed structure of the chromatin, allowing the RNA polymerase II complex to bind and initiate transcription. Id. Coactivators also recruit the chromatin remodeling SWI•SNF complex to target promoters. Id.

PPARγ, the most intensively studied PPAR isoform, exists in two protein isoforms that are created by alternative promoter usage and alternative splicing at the 5′ end of the gene; PPARγ2 contains 30 additional amino acids at the N terminus compared with PPARγ1. Id. Whereas many tissues express a low level of PPARγ1, PPARγ2 is fat-selective and is expressed at very high levels in that tissue.

Studies have shown that this receptor participates in biological pathways of intense basic and clinical interest, such as differentiation, insulin sensitivity, type 2 diabetes, atherosclerosis, and cancer. Id.

The thiazolidinediones (TZDs) or ‘glitazones’ are a class of oral antidiabetic drugs that improves metabolic control in patients with type 2 diabetes through the improvement of insulin sensitivity. (Hauner, H., “The mode of action of thiazolidinediones, Diabetes Metab. Res. Rev. 2: S10-15 (2002)). TZDs exert their antidiabetic effects through a mechanism that involves activation of PPAR γ. Id. TZD-induced activation of PPAR gamma alters the transcription of several genes involved in glucose and lipid metabolism and energy balance, including those that code for lipoprotein lipase, fatty acid transporter protein, adipocyte fatty acid binding protein, fatty acyl-CoA synthase, malic enzyme, glucokinase and the GLUT4 glucose transporter. Id. Although PPAR gamma is predominantly expressed in adipose tissue, TZDs reduce insulin resistance in adipose tissue, muscle and the liver. Id.

The PPARγ receptor plays a critical role in adipogenesis, and PPARγ binding is absolutely required for the function of the fat-selective enhancers for the aP2 and PEPCK genes in cultured fat cells. (Rosen, E D and Spiegelman, B M, “PPAR γ: a nuclear regulator of metabolism, differentiation, and cell growth,” J. Biol. Chem. 276: 37731-34 (2001)). In vivo, activation of this promoter in fat was shown to be dependent on a PPARγ binding site, whereas expression in other tissues was not. Id. Chimeric mice derived from both wild-type ES cells and cells with a homozygous deletion of PPARγ showed exclusion of null cells from white adipose tissue, but not several other tissues. Id. In vitro, it has also been shown that PPARγ is required for the differentiation of adipose cells from ES cells and from embryonic fibroblasts. Id.

There is evidence that PPARγ receptors can modulate the formation of foam cells in atherosclerotic plaques and that TZD treatment may be antiatherogenic. Id. Furthermore, because this receptor promotes differentiation, it is proposed that it may inhibit oncogenic effects in various cell types. Consistent with this, mutations and translocations of the PPARγ receptor have been identified in human tumors. Id.

PPARγ also induces the expression of proteins involved in reverse cholesterol transport, presumably leading to a net reduction of cholesterol in atherosclerotic lesions. Id. These transporters, ABCA1 and ABCG1, are actually induced by the orphan NHR LXRα, which is itself a target of PPARγ. Id. Telmisartan and irbesartan, two angiotensin type 1 receptor (AT1R) blockers (ARB), which have been found to activate PPARγ in cells derived from rodents independent of their At1R blocking action, have been reported to enhance both apoA-1 and HDL-mediated cholesterol efflux from macrophages by increasing ABCVA1, ABCG1 and SR-B1 expression via PPARγ-dependent and LXR-dependent/independent pathways. (Nakaya, K. et al., “Telmisartan enhances cholesterol efflux from THP-1 macrophages by activating PPARγ,” J. Atheroscler. & Thrombosis 14(3): 133-141 (2007)).

Cholesterol and sterol homeostasis is another regulatory system closely controlled by nuclear receptor function. The two major nuclear receptors involved in this regulatory system are FXR and LXR.

PPARγ and Cancer

Most liposarcomas have been found to express much higher levels of PPARγ than other sarcomas, and cells grown from liposarcomas were found to have a dramatic differentiation response to PPARγ ligands, including lipid accumulation, cessation of growth, and expression of mRNAs characteristic of fat differentiation. Id.

PPARγ is also expressed in a number of epithelial tissues that are important in human cancer, including breast, prostate, and colon. Id. PPARγ is expressed at very high levels in the colonic mucosa, comparable with the levels of expression in adipose tissue. Id. Application of synthetic ligands brings about a marked reduction in cell growth in large numbers of human colon cancer cell lines, and PPARγ activation results in alterations in patterns of gene expression favoring a more mature, less malignant phenotype. Id. Ligand administration to nude mice slows the growth of tumors derived from human colon cancer cells. Mutations of PPARγ in tumor tissue have been detected in some patients with adenocarcinoma of the colon; all mutations were heterozygous, and all involved loss of function of PPARγ, suggesting that PPARγ has tumor suppressor function in the human colon.

Paradoxically, administration of PPARγ ligands caused an increase in colon tumor number in Min mice, a mouse model of APC deficiency. Id. No increases in polyp number were seen in wild-type mice, nor have there been reports of PPARγ ligands causing increased tumor formation in humans. Id. These observations suggest that the role of PPARγ in the biology of the colon may be complex. Id.

PPARγ in the prostate may also play an important role in tumor suppression. Id. Up to 30% of patients with prostate cancer have heterozygous deletions of the 3p25 region containing PPARγ, although these deletions are rather large and include many genes. Id. In cultured prostate cell lines, TZDs have been shown to halt cell growth and to reduce secretion of the tumor marker PSA (prostate-specific antigen), and an encouraging response has been seen in some men with metastatic prostate cancer taking TZDs. Id

In some cases of follicular thyroid carcinoma, a fusion oncoprotein is formed by a chromosomal translocation between PAX8, deleted in its C-terminal activation domain, and full-length PPARγ1. Id. The resulting fusion protein, the expression of which in the thyroid is presumably driven by the PAX8 promoter, has an extremely powerful dominant negative activity on the transcriptional activity of wild-type PPARγ. Id. The addition of ligand does not relieve this dominant negative activity. Id. This translocation is not observed in benign follicular adenomas, suggesting that it is associated with carcinogenesis. Id. Although the contribution of both the PAX8 and PPARγ components are likely to be important, the crucial role of PPARγ as a tumor suppressor moiety in this oncoprotein is shown by the fact that other cases of this disease have a fusion protein formed between PPARγ and as yet unidentified partners. Id.

It has been reported that the survival rates and cell viabilities of A549 cells, as well as expression of ICAM-1 and MMP-9 were reduced by telmisartan in a time and concentration dependent manner. Li, J et al, “Telmisartan Exerts Anti-Tumor Effects by Activating Peroxisome Proliferator-Activated Receptor-γ in human lung adenocardinoma A549 Cells, Molecules 19: 2862-76 (2014)). These effects, as well as telmisartan-induced PPARγ activation were blunted or abrogated by the PPARγ antagonist GW9662. Id. It was concluded that telmisartan inhibited the expression of ICAM-1 and MMP9 in A549 cells, very likely through upregulation of PPARγ synthesis.

Farnesoid X Receptor (FXR)

The bile acid receptor (BAR), also known as farnesoid X receptor (FXR) or NR1H4 (nuclear receptor subfamily 1, group H, member 4) is a member of the nuclear receptor superfamily that is mainly expressed in liver, intestine, kidney and adipose tissue. (Claudel, T. et al, “The farnesoid X receptor, A molecular link between bile acid and lipid and glucose metabolism,” Arteriosclerosis, thrombosis, and vascular biology 25: 2020-30 (2005); Cariou, B., “The farnesoid X receptor (FXR) as a new target in non-alcoholic steatohepatitis,” Diabetes Metab. 34(6 pt. 2): 685-91 (2008)). FXR is highly expressed in liver and intestine. Claudel, T. et al, “The farnesoid X receptor, A molecular link between bile acid and lipid and glucose metabolism,” Arteriosclerosis, thrombosis, and vascular biology 25: 2020-30 (2005). On activation by bile acids, FXR regulates a wide variety of target genes that are critically involved in the control of bile acid, lipid and glucose homeostasis. Id.

FXR is a biosensor for endogenous bile acids. (Kemper, J K, “Regulation of FXR transcriptional activity in health and disease: emerging roles of FXR cofactors and post-translational modifications,” Biochim. Biophys. Acta 1812 (8): 842-50 (2011)). A primary bile acid, chenodeoxycholic acid (CDCA) is the most potent natural FXR agonist, with a half maximal effective concentration (EC50) value of about 10 μM. Id. Secondary bile acids, lithocholic acid (LCA) and deoxycholic acid (DCA) also activate FXR, but to a lesser extent. Id.

After ligand binding, FXR, either as a monomer, or as a FXR/RXR (meaning retinoid X receptor) heterodimer, binds to, and either activates or represses, DNA elements called FXR response elements (FXREs) and activates transcription of target genes. Id.

FXR activation inhibits hepatic de novo lipogenesis, increases insulin sensitivity and protects hepatocytes against bile acid-induced cytotoxicity. (Claudel, T. et al, “The farnesoid X receptor, A molecular link between bile acid and lipid and glucose metabolism,” Arteriosclerosis, thrombosis, and vascular biology 25: 2020-30 (2005); Cariou, B., “The farnesoid X receptor (FXR) as a new target in non-alcoholic steatohepatitis,” Diabetes Metab. 34(6 pt. 2): 685-91 (2008)).

FXR and Bile Acid Metabolism

Bile acids are synthesized from cholesterol exclusively by the liver. The biosynthetic steps that collectively accomplish the conversion of water-insoluble cholesterol molecules into more water-soluble compounds also confer detergent properties to the bile acids that are crucial for their physiological functions in bile formation and intestinal fat absorption. On conjugation with glycine or taurine, bile acids are actively secreted by the hepatocytes into the bile canaliculi that drain into intrahepatic bile ducts, stored in the gall bladder, and expelled into the intestinal lumen in response to a fatty meal. In the small intestine, bile acids act as detergents to emulsify and facilitate the absorption of dietary fats and lipid-soluble vitamins. Subsequently they are reabsorbed from the terminal ileum by specific transporter proteins.

As shown in FIG. 2, two major pathways (the neutral and the acidic pathway) are involved in bile acid synthesis. CYP7A1 is the first and rate controlling enzyme of the neutral pathway and is partly controlled by a negative bile acid feedback loop. Id. CYP27A1, the main enzyme of the acidic pathway, is not regulated by bile acids. Id.

FXR activation induces expression of the atypical nuclear receptor small heterodimer partner (SHP or NR0B2), which interacts with two other nuclear receptors that transactivate CYP7A1 expression via the bile acid response element (BARE) region, i.e., the hepatic nuclear factor 4 (HNF4 or NR2A1) and the liver receptor homolog-1 (LRH-1 or NR5A2). Id. SHP repression of CYP7A1 gene transcription occurs by promoting the dissociation of coactivators linked to HNF4 and LRH-1, as well as by histone deacetylation of the promoter. Id.

FXR also modulates CYP7A1 expression by induction of fibroblast growth factor-19 (FGF-19) expression. Id. On its secretion, FGF-19 activates the hepatic FGF receptor-4, which, in turn, downregulates CYP7A1 through C-Jun N-terminal kinase activation. Id.

Bile acids are conjugated to taurine or glycine by sequential actions of the enzymes bile acid coenzyme A (CoA) sythetase (BACS) and the bile acid-CoA amino acid N-acetyltransferase (BAT) to increase their hydrophilicity in a process regulated by FXR. Id. Conjugated bile acids require a transporter network to cycle between liver and intestine, which is, to a certain extent also under FXR control. Id. Bile acids are secreted by hepatocytes into the bile canaliculi by the bile salt export pump (BSEP or ABCB22) via an ATP-dependent process. BSEP expression is induced by FXR at the transcriptional level. Id. Because relatively hydrophobic bile acids are potentially toxic, protective mechanisms such as oxidation by CYP3A4, sulfation by dehydroepiandrosterone-sulfotransferase (SULT2A1), or glucuronidation catalyzed by uridine glucuronosyltransferase 2B4 (UGT2B4) have evolved; CYP3A4, SULT2A1, and UGT2B4 are all positively regulated by FXR by means of a nonclassical inverted repeat 0 (IR-0), a monomeric site, and two response elements (an ER-8 and another IR-1/DR-3 site), respectively. Id. FXR also induces the expression of the multidrug resistance-associated protein-2 (MRP2 or ABCC2), a multispecific ABC transporter able to excrete sulfated and glucuronidated bile acids into the bile, via an everted repeat-8 (ER-8) site.

In the ileum, bile acids are efficiently taken up by enterocytes via the ileal apical sodium-dependent bile acid transporter (ASBT, also called intestinal bile acid transporter (IBAT) or SLC10A2) protein. Id. FXR indirectly influences the expression of ASBT, but directly induces the expression of the intestinal bile acid binding protein (IBAB-P or FABP6). Id. FXR deficient mice were found to display enhanced intestinal bile acid absorption despite an extremely low IBAB-P expression. Id.

The uptake of bile salts that return to the liver after intestinal absorption is mainly mediated by the Na+ taurocholate cotransporting polypeptide (NTCP or SLC10A1). Id. Bile acids downregulate NTCP expression via a FXR-dependent mechanism, but NTCP expression is not changed in FXR-deficient mice, compared with wild type controls. Id.

FXR and Cholesterol Metabolism

It has been suggested that FXR may play an important role in regulating blood levels of asymmetric dimethylarginine (ADMA), a potent endogenous inhibitor of endothelial nitric-oxide synthase (eNOS), via coordinated regulation of dimethylarginine dimethylaminohydrolase-1 (DDAH) and cationic amino acid-1 (CAT-1) in liver and kidney. Li, J. et al, “Coordinated regulation of dimethylarginine dimethylaminohydrolase-1 and cationic amino acid transporter-1 by farnesoid X receptor in mouse liver and kidney and its implication in the control of blood levels of asymmetric dimethylarginine,” J. Pharmcol. & Exptl Therapeutics 331 (10: 234-43 (2009)). Circulating ADMA is largely taken up by liver and kidney via system y⁺ carriers of the cationic amino acid (CAT) family and subsequently metabolized by DDAHs. Id. Treatment of mice with an FXR agonist 3-(2,6-dichlorophenyl)-4-(3′-carboxy-2-chlorostilben-4-yl)oxymethyl-5-isopropylisoxazole; GW4064) led to increased expression of DDAH-1 and CAT-1 in both liver and kidney. In cultured human hepatocytes and kidney proximal tubular epithelial cells, GW4064 increased CAT-1 expression, and this was associated with a significant increase in the cellular uptake of ADMA. Promoter analyses suggested that CAT-1 is a likely target of FXR, and a functional FXR response element was found in the promoter region of CAT-1 gene.

FXR and Triglyceride Metabolism

FXR controls a variety of genes crucially involved in triglyceride metabolism in the blood compartment. Id. FXR also regulates the expression of peroxisome proliferator-activated receptor (PPAR)-α in humans. Id. PPARα is a nuclear receptor that is activated by fatty acids and by fibrates, a class of synthetic hypolipidemic drugs. Id. PPARα activation also decreases plasma triglyceride levels. Id.

FXR and Glucose Homeostasis

FXR has been identified as a gene positively regulated by glucose; in cultured hepatocytes, glucose was shown to induce Fxr gene expression, probably via metabolites of the pentose phosphate pathway, whereas insulin counter-regulated this effect. Id. Moreover, apoC-III gene expression was additively repressed by glucose and the synthetic FXR agonist GW4064 in cultured cells and Cyp7A1 gene expression was inversely correlated with Fxr gene expression in livers of diabetic rats. Id.

In vitro and in vivo data have shown that bile acids modulate gluconeogenesis by regulating the expression of the rate controlling enzyme phosphoenol pyruvate carboxykinase (PEPCK) as well as of glucose-6-phosphatase (G6Pase) and fructose-1,6-bisphosphatase (FBP1). Id. FXR was not required to downregulate PEPCK expression (incubation of cells with bile acids reduced hepatic nuclear factor 4α (HNF4α) transactivation of the PEPCK promoter while a synthetic FXR agonist did not). HNF4α is a major regulator of PEPCK expression, a master regulator of hepatic gene expression, and is also involved in the maturity onset diabetes of the young type 1 (MODY1). Id.

During the fasting period when gluconeogenesis is induced Cyp7A1, Pepck, Pparα, and Fxr gene expression are upregulated. Id. Because Fxr-repressed genes, such as Cyp7A1 and Pepck, are upregulated, this implies that FXR control is weak in this condition, probably because bile acids are not circulating and are stored in the gall bladder. Id. At the same time, Shp gene expression is not changed during fasting. Id.

FXR Cofactors

The induction of FXR and the bioavailability of its ligands together may be necessary in obtaining a physiological effect on gene expression.

Exemplary transcriptional cofactors that modulate FXR transactivation ability are shown in table 2 (Kemper, J K, “Regulation of FXR transcriptional activity in health and disease: emerging roles of FXR cofactors and post-translational modifications,” Biochim. Biophys. Acta 1812 (8): 842-50 (2011)). In general, interactions of FXR with coactivators, including Brg-1, p300, CARM1, PRMT1, and ASCOM, are increased after treatment with FXR agonists, and the coactivators are recruited to FXR target genes, which results in histone modification or chromatin remodeling and increased gene transcription. Id. Some of these cofactors, including p300 and SIRT1, acetylate and deacetylate not only histones at FXR target genes, but also FXR itself.

TABLE 2
Transcriptional cofactors of FXR

| Cofactor | Cellular Function | Regulation of FXR signaling |
|---|---|---|
| PGC-1α | Critical metabolic regulator in mitochondrial | Transcriptional coactivator |
| | biogenesis and function | of FXR; increases |
| | | transcription of Fxr gene by |
| | | coactivation of PPARγ and |
| | | HNF-4 |
| p300 | Catalyzes covalent modification of acetylation | Coactivator for FXR in SHP |
| | at lysine residues in histones and | gene induction by catalyzing |
| | transcriptional factors/cofactors | acetylation of K9/K14 of |
| | | histone H3; also acetylates |
| | | FXR |
| SRC-1 | General NR AF2 coactivator of Lysine | Increases FXR |
| | acetylase | transactivation |
| SIRT1 | NAD+-dependent deacetylase that removes | Increases transactivation |
| | acetyl groups from modified lysines in | potential of FXR by |
| | histones and nonhistone regulatory proteins | deacetylating FXR; also |
| | | deacetylates histones at the |
| | | SHP promoter which is |
| | | expected to maintain |
| | | transcriptional silencing. |
| Brg-1 | ATPase of Swif/Snf complexes | FXR coactivator for SHP |
| | | gene induction. |
| CARM1/PRMT4 | PRMT | Interacts with and enhances |
| | | transactivation ability of |
| | | FXR: increased occupancy |
| | | of CARM1 at the BSEP |
| | | promoter, an FXR target |
| | | gene that functions as an |
| | | ATP-dependent canalicular |
| | | bile acid transporter, |
| | | resulted in increased |
| | | methylation art Art-17 of |
| | | histone H3 and gene |
| | | activation. |
| PRMT1 | PRMT1 | Treatment with FXR agonist |
| | | 6E-CDCA increased |
| | | interaction of FXR with |
| | | PRMT1 and increased |
| | | mRNA levels of FXR target |
| | | genes BSEP and SHP. Both |
| | | the occupancy of PRMT 1 |
| | | and histone H4 methylation |
| | | at the promoters of the |
| | | BSEP and SHP genes were |
| | | increased. |
| ASCOM | A coactivator complex containing activating | ASC-2, MLL3 and MLL4 |
| | signal cointegrator-2 (ASC-2) and H3K4 Lys | were recruited to FXR target |
| | methyltransferases (MLL3/4) | genes in a ligand-dependent |
| | | manner, resulting in H3K4 |
| | | trimethylation and gene |
| | | activation. |
| GPS2 | A subunit of the N-CoR corepressor complex | Regulates CYP8B1, a direct |
| | | FXR target gene. GPS2 |
| | | augment expression of the |
| | | gene. |
| Ku proteins | DNA-dependent protein kinase catalytic | FXR-interacting proteins |
| (Ku70 and | subunits | that function as |
| Ku80) | | corepressors, suggesting that |
| | | phosphorylation of FXR |
| | | may be an important post- |
| | | translational modification. |
| DRIP205 | Transcription coactivator of FXR | Augment transactivation |
| | | ability of FXR. |
| TRRAP | Transcription coactivator of FXR | Augment transactivation |
| | | ability of FXR. |

*Taken from Kemper, J K, “Regulation of FXR transcriptional activity in health and disease: emerging roles of FXR cofactors and post-translational modifications,” Biochim. Biophys. Acta 1812 (8): 842-50 (2011) (citations omitted).

FXR and Enterohepatic Cancers

FXR is expressed predominantly in tissues exposed to high levels of bile acids and controls bile acid and lipid homeostasis. (Deuschle, U. et al, “FXR Controls the Tumor Suppressor NDRG2 and FXR Agonists Reduce Liver tumor growth and metastasis in an orthotopic mouse xenograft model,” PLoS/ONE 7 (10): e43044, doi: 10.1371/journal.poine.0043044)). FXR−/− mice develop hepatocellular carcinoma (HCC) and show an increased prevalence for intestinal malignancies, suggesting a role of FXR as a tumor suppressor in enterohepatic tissues. Id.

The N-myc downstream-regulated gene 2 (NDRG2) has been recognized as a tumor suppressor gene, which is downregulated in human hepatocellular carcinoma, colorectal carcinoma and many other malignancies. Id. Reduced NDRG2 mRNA has been shown in livers of FXR−/− mice compared to wild type mice, and both FXR and NDRG2 mRNAs, are reduced in human HCC compared to normal liver. Id. Gene reporter assays and Chromatin Immunoprecipitation data support that FXR directly controls NDRG2 transcription via IR1-type element(s) identified in the first introns of the human, mouse and rat NDRG2 genes. Id. NDRG2 mRNA was induced by non-steroidal FXR agonists in livers of mice and the magnitude of induction of NDRG2 mRNA in three different human hepatoma cell lines was increased when ectopically expressing human FXR. Id. Growth and metastasis of SK-Hep-1 cells was strongly reduced by non-steroidal FXR agonists in an orthotopic liver xenograft tumor model. Id. Ectopic expression of FXR in SK-Hep1 cells reduced tumor growth and metastasis potential of corresponding cells and increased the anti-tumor efficacy of FXR agonists, which may be partly mediated via increased NDRG2 expression. Id.

Liver X Receptors (LXRs)

Liver X receptors (LXRs) are ligand-activated transcription factors of the nuclear receptor superfamily. (Baranowski, M., “Biological role of liver X receptors,” J. Physiol. Pharmacology. 59 Suppl. 7: 31-55 (2008)). There are two LXR isoforms termed alpha and beta which upon activation form heterodimers with retinoid X receptor and bind to LXR response element found in the promoter region of the target genes. Id. High expression levels of LXRα in metabolically active tissues fit with central role of the receptor in lipid metabolism, while LXRβ is more ubiquitously expressed. (Pehkonen, P. et al., “Genome-wide landscape of liver X receptor chromatin binding and gene regulation in human macrophages,” BMC Genomics 13: 50 (2012)). Both LXRs are found in various cells of the immune system, such as macrophages, dendritic cells and lymphocytes. Id. In macrophages, the accumulation of excess lipoprotein-derived cholesterol activates LXR and triggers the induction of a transcriptional program for cholesterol efflux, such as ATP-binding cassette transporter (ABC) A1 (ABCA1) and ABCG1, while in parallel the receptor transrepresses inflammatory genes, such as inducible nitric oxide synthase, interleukin 1β, and monocyte chemotactic protein-1. Id.

Endogenous agonists of the LXRs include a variety of oxidized cholesterol derivatives referred to as oxysterols. (Baranowski, M., “Biological role of liver X receptors,” J. Physiol. Pharmacol. 59 Suppl. 7: 31-55 (2008)). LXRs have been characterized as key transcriptional regulators of lipid and carbohydrate metabolism. Id. LXRs were shown to function as sterol sensors protecting the cells from cholesterol overload by stimulating reverse cholesterol transport and activating its conversion to bile acids in the liver. Id. This finding led to identification of LXR agonists as potent anti-atherogenic agents in rodent models of atherosclerosis. Id. However, first-generation LXR activators were also shown to stimulate lipogenesis via sterol regulatory element binding protein-1c leading to liver steatosis and hypertriglyceridemia. Id.

Despite their lipogenic action, LXR agonists possess antidiabetic properties. Id. LXR activation normalizes glycemia and improves insulin sensitivity in rodent models of type 2 diabetes and insulin resistance. Id. Although antidiabetic action of LXR agonists is thought to result predominantly from suppression of hepatic gluconeogenesis, some studies suggest that LXR activation may also enhance peripheral glucose uptake. Id.

Published reports of anti-proliferative effects of synthetic LXR ligands on breast, prostate, ovarian, lung, skin, and colorectal cancer cells suggest that LXRs are potential targets in cancer prevention and treatment. Nguyen-Vu, T. et al, “Liver x receptor ligands disrupt breast cancer cell proliferation through an E2F-mediated mechanism,” Breast Cancer Res. 15: R51 (2013). Cell line-specific transcriptional responses and a set of common responsive genes were shown by microarray analysis of gene expression in four breast cell lines [MCF-7 (ER+), T-47D (ER+), SK-BR-3 (ER−), and MDA-MB-231] following treatment with the synthetic LXR ligand GW3965. Id. In the common responsive gene set, upregulated genes tend to function in the known metabolic effects of LXR ligands and LXRs whereas the downregulated genes mostly include those which function in cell cycle regulation, DNA replication, and other cell proliferation-related processes. Id. Transcription factor binding site analysis of the downregulated genes revealed an enrichment of E2F binding site sequence motifs. Id. Correspondingly, E2F2 transcript levels are downregulated following LXR ligand treatment. Id. Knockdown of E2F2 expression, similar to LXR ligand treatment, resulted in a significant disruption of estrogen receptor positive breast cancer cell proliferation. Id. Ligand treatment also decreased E2F2 binding to cis-regulatory regions of target genes.

Expression of activated LXRα blocks proliferation of human colorectal cancer cells and slows the growth of xenograft tumors in mice, and reduces intestinal tumor formation after administration of chemical carcinogens, and in Apc(min/+) mice. Lo Sasso, G. et al., “Liver X receptors inhibit proliferation of human colorectal cancer cells and growth of intestinal tumors in mice,” Gastroenterology 144(7): 1497-507 (2013). A link of LXRs to apoptosis has been reported. (Pehkonen, P. et al, “Genome-wide landscape of liver X receptor chromatin binding and gene regulation in human macrophages,” BMC Genomics 13: 50 (2012)).

PRMTs as Modulators of Nuclear Receptors

PRMT1 has been reported to play a regulatory role independent of methyltransferase activity, in nuclear receptor TR3 transactivation. Lei, N et al, J. Biol. Chem. 276 (41): 37731-34 (2001), Unlike another orphan receptor HNF4, TR3 is not methylated by PRMT1 although they physically interact with each other. Id. By delaying the TR3 protein degradation, PRMT1 binding leads to the elevation of TR3 cellular protein level, thereby enhancing the DNA binding and transactivation activity of TR3 in a non-methyltransferase manner. Id. Another coactivator SRC-2 acts synergistically with PRMT1 to regulate TR3 functions. Id. In turn, TR3 binding to the catalytic domain of PRMT1 causes an inhibition of the PRMT1 methyltransferase activity. Id. This repression results in the functional changes in some of PRMT1 substrates, including STATS and Sam68. Id. The negative regulation of PRMT1 by TR3 was further confirmed in both TR3-knockdown cells and TR3-knockout mice with the use of an agonist for TR3. Id.

PRMT2 has been reported to be an estrogen receptor (ER)α coactivator. Qi, C. et al., “Identification of PRMT2 as a coactivator for estrogen receptor α,” J. Biol. Chem. 277 (32): 28624-30 (2002)). PRMT2 interacted directly with three ERα regions including AF-1, DNA binding domain and hormone binding domain in a ligand-independent fashion. The ERα interacting region on PRMT2 was mapped to a region encompassing amino acid 133 to 275. PRMT2 also binds to ERβ, PR, TRβ, RARα, PPARγ, and RXRα in a ligand-independent manner. PRMT2 enhanced both ERα AF-1 and AF-2 transcriptional activity and the potential methyltransferase activity of PRMT2 appeared pivotal for its coactivator function. In addition, PRMT2 enhanced PR, PPARγ and RARα mediated transactivation. Although PRMT2 was found to interact with other two coactivators SRC-1 (steroid receptor coactivator-1) and PRIP (peroxisome proliferator activated receptor interacting protein), unlike coactivator PRMT1 and CARM1, no synergistic enhancement of ERα transcriptional activity was observed when PRMT2 was co-expressed with either PRIP or SRC-1.

The pathophysiological role of PRMT3 in nonalcoholic fatty liver disease (NAFLD) and its relationship with LXRα was examined by Kim, D I et al, Diabetes 64 (1): 60-71 (2015). LXRα and PRMT3 expression was increased in cellular and mouse models of NAFLD and human patients, and PRMT3 translocated into the nucleus bound with LXRα as a transcriptional cofactor, which induced lipogenesis. In conclusion, PRMT3 translocation by PA is coupled to the binding of LXRα, which is responsible for the onset of fatty liver.

Members of the p160 coactivator family (steroid receptor coactivator-1 (SRC-1), glucocorticoid receptor interacting protein 1 (GRIP1), and activator of thyroid and retinoic acid receptors (ACTR)) mediate transcriptional activation by nuclear receptors Chen, D. et al, “Synergistic, p160 coactivator-dependent enhancement of estrogen receptor function by CARM1 and p300,” J. Biol. Chem. 275 (52): 40810-6 (2000). After being recruited to the promoter by nuclear receptors, the p160 coactivator transmits the activating signal via two C-terminal activation domains, AD1 and AD2. Id. AD1 is a binding site for the related coactivators cAMP-response element binding protein binding protein (CBP) and p300, whereas AD2 binds to another coactivator, CARM1. Id. The cooperative functional and mechanistic relationships among GRIP1, CARM1 and p300 were determined in transient transfection assays, where they enhanced the ability of the estrogen receptor (ER) to activate transcription of a reporter gene. Id. The coactivator functions of p300 and CARM1 depended on the co-expression of GRIP1. Id. Simultaneous co-expression of all three coactivators caused a synergistic enhancement of ER function. Id. Deletion of the AD1 domain of GRIP1 abolished the ability of p300 to potentiate ER activity but had no effect on CARM1-mediated stimulation. Id. In contrast, when the AD2 domain of GRIP1 was deleted, p300 still stimulated ER function through the mutant GRIP1, but CARM1 failed to do so. Id. Thus, both binding of p300 to AD1 and binding of CARM1 to AD2 are required for their respective coactivator functions and for their synergy. Id. Furthermore, CARM1 and p300 function independently through different activating domains of GRIP1, and their synergy suggests that they enhance transcription by different, complementary mechanisms. Id.

The multiple LIM domain protein Ajuba is a unique corepressor for a subset of nuclear hormone receptors. Ajuba belongs to the Ajuba/Zyxin family of LIM proteins, which includes Ajuba, Limd1, WTIP, Zyxin, LPP, and Trip6. Hou, Z. et al., “LIM Protein Ajuba functions as a nuclear receptor corepressor and negatively regulates retinoic acid signaling,” Proc. Natl Acad. Sci. USA. 107 (7): 2938-43 (2010). The family, which functions as scaffolds, participating in the assembly of numerous protein complexes, is characterized by two or three tandem C-terminal LIM domains and a unique N-terminal prelim region. Id. Ajuba has been shown to bind Grb2 to modulate serum-stimulated ERK activation, to recruit the TNF receptor associated factor 6 (TRAF6) to p62 and activates PKCζ activity; to interact with α-catenin and F-actin to contribute to the formation or stabilization of adheren junctions by linking adhesive receptors to the actin cytoskeleton; to form a complex with the p130Case/Dock180 Rac guanine nucleotide-exchange factor to regulate Rac activity, and to bind the Aurora A kinase to regulate mitosis. Id. In addition, Ajuba has been shown to contain functional nuclear receptor interacting motifs; to function as a corepressor for the zinc finger-protein Snail, and to bind to the SNAG repression domain of Snail through its LIM region. Id. PRMT5 has been identified as an effector recruited to Snail through an interaction with Ajuba. Id. The Snail, Ajuba, PRMT5 complex functions to repress E-cadherin, a known Snail target gene; Snail, Ajuba and PRMT5 can be found at the proximal promoter region of the E-cadherin gene concomitant with increased arginine methylation of histones at this locus. Id.

Ajuba has been shown to play a significant role in the regulation of mouse embryonic carcinoma P19 cell proliferation and differentiation, a process highly dependent upon retinoids. Id. Ajuba selectively interacts with retinoic acid receptors (RARs) and rexinoid receptor (RXRs) subtypes in a ligand-dependent manner. Id. Simultaneous mutation of these motifs abolishes RAR binding and concomitantly leads to loss of repression on RARE reporter genes. Id. Coexpression of the Ajuba binding partner PRMT5 inhibited the Ajuba-RAR interaction. Id. The high-affinity Ajuba-RAR/RXR interaction site overlaps the region responsible for Ajuba/PRMT5 binding.

PRMTs and Disease

Pulmonary Diseases

Protein arginine methylation, either by direct regulation of protein function or by metabolic products originating from protein arginine methylation that influence nitric oxide (NO)-dependent processes, has been implicated in cardiovascular and pulmonary diseases, including lung cancer, pulmonary fibrosis, pulmonary hypertension, chronic obstructive pulmonary disease, and asthma. (Zakrzewicz, D et al, “Protein arginine methyltransferases (PRMTs): promising targets for the treatment of pulmonary disorders,” Intl. J. Mol. Seci. 13: 12383-400 (2012)).

Monomethylarginine and asymmetric dimethylarginine are potent inhibitors of nitric oxide synthase (NOS), and increased levels of these have been found in patients with various cardiovascular and noncardiovascular disorders. (Lim S K et al, “Activation of PRMT1 and PRMT5 mediates hypoxia- and ischemia-induced apoptosis in human lung epithelial cells and the lung of miniature pigs: the role of p38 and JNK mitogen-activated protein kinases,” Biochem. Biophys. Res. Commun. 440(4): 707-13 (2013)).

In A459 human lung epithelial cells, hypoxia increases the expression of PRMT1 and PRMT5, and overexpression of PRMT1 and PRMT5 induces apoptosis (Id). Hypoxia-induced expression of PRMT1 and PRMT5 was blocked by p38 and JNK mitogen-activated protein kinase (MAPK) inhibitors, but not by an inhibitor of extracellular signal-regulated kinases (ERK)1/2. Id. In the lungs of miniature pigs, ischemia stimulated PRMT1 and PRMT5 expression and induced phosphorylation of p38 MAPK, phosphorylation of JNK and apoptotic molecules. Id.

Dalloneau et al found in mice that Prmt2 is a dosage-sensitive gene contributing to the control of the lipopolysaccharide (LPS)-induced inflammatory response in lungs and macrophages by regulating the nuclear level of NF-κB. Id. (Dalloneau, E., et al, “Prmt2 regulates the lipopolysaccharide-induced responses in lungs and macrophages,” J. Immunol. 2011, doi: 10.4049/jimmunol.1101087)). LPS-induced inflammation is the consequence of the activation of the reticuloendothelial system, leading to synthesis of various mediators, including the principal proinflammatory cytokines IL-1, Il-6, and TNF-α. Id. Expression of IL-6 and TNF-α (which are responsible for the amplification of the inflammatory response) is directly regulated by NF-κB. Id. PRMT2 is known to act as a negative regulator of the NF-κB pathway in a dose dependent manner. Id.

### Metabolic Diseases

PRMT1 and dimethylarginine dimethylaminohydrolase 1 and 2 (DDAH1/2) can determine the bioavailable cellular levels of ADMA and subsequent local NO levels. (Iwasaki, H., “Activities of asymmetric dimethylarginine-related enzymes in white adipose tissue are associated with circulating lipid biomarkers,” Diabetology & Metabolic syndrome 4: 17 (2012)). ADMA, a naturally occurring inhibitor of NOS, is produced by the proteolysis of intracellular proteins that are postranslationally modified by PRMT1. DDAH1/2 catalyze the hydrolysis of ADMA into L-citrulline and dimethylamine. (Id., citing Palm, F. et al, “Dimethylarginine dimethylaminohydrolase (DDAH): expression, regulation and function in the cardiovascular and renal systems,” Am. J. Physiol. Heart Cir. Physiol. 293: H3227-H3245 (2007)). The expression and catalytic activity of PRMT1 and DDAH1/2 in the white adipose tissue (periepididymal, visceral and subcutaneous fats) and femur skeletal muscle tissue were determined in a rodent model of type 2 diabetes mellitus, characterized by markedly increased blood flow in white adipose tissue with the considerable contribution of the NOS/NO system, and compared to control rats by immunoblotting, in vitro methyltransferase assays and in vitro citrulline assays. Id. It has been reported that liver and pancreatic lysates from GK rats display low PRMT1 activity, and that this may be associated with defective hepatic insulin signaling, excessive gluconeogenesis in the liver, and inappropriate glucose-stimulated insulin secretion. Id. PRMT1 and DDAH activities were altered in the diabetic Goto-Kakizaki (GK) rats in an organ specific manner, which was reflected in the serum levels of serum non-esterified fatty acids (NEFA) and triglycerides (TG). Id. The non-obese diabetic GK rats showed lower expression and activity of adipose PRMT1 compared to age match controls. Id. Adipose tissues of GK rats had high DDAH1 and expression and total DDAH activity, whereas DDAH2 expression was below the control value. Id. GK rats had lower levels of NEFA and TG than controls. In all subjects the adipose PRMT1 and DDAH activities were statistically correlated with the levels of NEFA and TG in serum. Id. The mechanism(s) for the modification of PRMT1 and DDAH1/2 expression in the adipose tissue of GK rats is not understood.

Cardiomyopathy

PRMT5 is highly expressed in the heart. It has been shown to specifically interact with transcriptional factor GATA4 in both co-transfected HEK293T cells and neonatal rat cardiomyocytes. (Chen, M. et al, “Inhibition of cardiomyocyte hypertrophy by protein arginine methyltransferase 5,” J. Biol. Chem. 289 (35): 24325-35 (2014)). In the heart, GATA4 has multiple distinct roles in cardiac specification, differentiation, morphogenesis, hypertrophy and survival. (Gallagher, J M, et al, “Carboxy terminus of GATA4 transcription factor is required for its cardiogenic activity and interaction with CD4,” Science Direct 134: 31-41 (2014)). In the adult mammalian heart, GATA4 mediates cardiomyocyte hypertrophy in a process thought to resemble aspects of cardiac development, as suggested by activation of a set of cardiomyocyte gene products that are normally expressed in developing, immature cells and in cells undergoing hypertrophy. Id.

The interaction of PRMT4 with GATA4 leads to the arginine methylation of GATA4 at positions 229, 265 and 317, which leads to an inhibition of GATA4 transcriptional activity, predominantly through blocking the p300-mediated acetylation of GATA4 in cardiomyocytes. (Chen, M. et al, “Inhibition of cardiomyocyte hypertrophy by protein arginine methyltransferase 5,” J. Biol. Chem. 289 (35): 24325-35 (2014)). Overexpression of PRMT5 substantially inhibited the acetylation of GATA4 and cardiac hypertrophic responses in phenylephrine-stimulated cardiomyocytes, whereas knockdown of PRMT5 induced GATA4 activation and cardiomyocyte hypertrophy. Id. In response to phenylephrine stimulation, PRMT5 translocates into the cytoplasm, thus relieving its repression on GATA4 activity in the nucleus and leading to hypertrophic gene expression in cardiomyocytes. Id.

PRMTs and Cancers

Aberrant expression of PRMTs has been observed in several cancers, including breast, lung, colorectal, bladder, mantle cell lymphoma, cancers of the brain and leukemia. (Baldwin, R. M. et al., “Alternatively spliced protein arginine methyltransferase 1 isoform PRMT1v2 promotes the survival and invasiveness of breast cancer cells,” Cell Cycle 1 (24): 4597-4612 (2012)). For the most part it is not known whether the PRMT dependent alterations are the cause or the result of the pathological changes observed.

For example,

PRMT1 has been shown to be overexpressed in colorectal cancer and, along with PRMT6, is overexpressed in lung and bladder cancers. (Baldwin, R. M. et al., “Alternatively spliced protein arginine methyltransferase 1 isoform PRMT1v2 promotes the survival and invasiveness of breast cancer cells,” Cell Cycle 1 (24): 4597-4612 (2012)), citing Kim, Y R et al, “Differential CARM1 expression in prostate and colorectal cancers,” BMC Cancer 10: 197 (2010); Mathioudaki, K. et al, “The PRMT1 gene expression pattern in colon cancer,” Br. J. Cancer 99: 2094-99 (2004); Yoshimatsu, M. et al., “Dysregulation of PRMT1 and PRMT6 Type 1 arginine methyltransferases is involved in various types of human cancers,’ Intl J. Cancer 128: 562-73 (2011)); has been shown to be an important component of a novel oncogenic transcriptional complex with mixed lineage leukemia (MLL) and can regulate MLL-mediated cell transformation (Id., citing Cheung, N. et al, “Protein arginine-methyltransferase dependent oncogenesis,” Nature Cell Biol. 9: 1208-15 (2007)); and also can methylate the estrogen receptor (ER) in breast cancer cells, which promotes ER extranuclear survival signaling and interaction with mediators of cell migration. (Id., citing Le Romancer, M. et al., “Regulation of estrogen rapid signaling through arginine methylation by PRMT1,” Mol. Cell 31: 212-21 (2008)). Moreover, reduced PRMT6 expression was associated with better overall relapse-free and distant metastasis-free survival in breast cancer patients with the estrogen receptor (ER (ESR1))-positive invasive ductile carcinoma, supporting the notion that PRMT6-dependent transcription and alternative splicing may also be involved in lung cancer pathophysiology. (Zakrzewicz, D. et al, “Protein arginine methyltransferases (PRMTs): promising targets for the treatment of pulmonary disorders,” Intl J. Molec. Sci. 13: 12383-400 (2012), citing Dowhan, D H, et al, “Protein arginine methyltransferase 6-dependent gene expression and splicing: association with breast cancer outcomes,” Endocr. Relat. Cancer 19: 509-26 (2012)).

A specific role for PRMT1v2, the predominantly cytoplasmic isoform of PRMT1, in breast cancer survival and invasion has been suggested. Id. This activity required both proper subcellular localization and methylase activity. Id. Specific depletion of PRMT1v2 using RNA interference caused a significant decrease in cancer cell survival due to induction of apoptosis. Id. Depletion of PRMT1v2 in an aggressive cancer cell line significantly decreased cell invasion, and overexpression of PRMT1v2 in a non-aggressive cancer cell line was sufficient to render the cells more invasive. Id. PRMT1v2 overexpression altered cell morphology and reduced cell-cell adhesion, which was linked with reduced β-catenin protein expression. Id.

CARM1 and PRMT1 have been shown to be significantly overexpressed in patients with non-small cell lung carcinomas (NSCLC) and in two NSCLC cell lines, A549 and H1299. Only CARM1 expression was found to be correlated with tumor differentiation, and neither CARM1 nor PRMT1 expression was correlated with survival. CARM1 targeting suppresses PRMT1 in addition to CARM1. (Elakoum, R. et al, “CARM1 and PRMT1 are dysregulated in lung cancer without hierarchical features,” Biochimie 97: 210-8 (2014)).

PRMT4/CARM1 expression is elevated in prostate and breast cancer, and is thought to play a role in the regulation of hormone-dependent proliferation (Id., citing Hong, H et al, “Aberrant expression of CARM1, a transcriptional coactivator of androgen receptor, in the development of prostate carcinoma and androgen-independent status,” Cancer 101: 83-89 (2004); Frietze, S. et al, “CARM1 regulates estrogen-stimulated breast cancer growth through up-regulation of E2F1,” Cancer Res. 68: 301-6 (2008)).

PRMT5 expression is upregulated in mantle cell lymphoma and enhances anchorage-independent cell growth. (Id., citing Pal, S. et al, “Low levels of miR-92b/96 induce PRMT5 translation and H3R8/H4R3 methylation in mantle cell lymphoma,” EMBO J. 26: 3558-69 (2007); Pal, S. et al., “Human SWI/SNF-associated PRMT5 methylates histone H3 arginine 8 and negatively regulates expression of ST7 and NM23 tumor suppressor genes,” Mol. Cell Biol. 24: 9630-45 (2004)) It has also been shown that PRMT5 is involved in tumor formation by upregulating G1 cyclins/cyclin-dependent kinases and the PI3K/AKT signaling cascade in lung cancer cell lines A549 and H1299. (Wei, T Y et al., “Protein arginine methyltransferase 5 is a potential oncoprotein that upregulates G1 cyclins/cyclin-dependent kinases and the phosphoinositide 3 kinase/AKT signaling cascade,” Cancer Sci. 103: 1640-50 (2012)).

PRMT5 also can antagonize pro-apoptotic signaling pathways. Karkhanis, V. et al, “Versatility of PRMT5-induced methylation in growth control and development,” Trends Biochem. Sci. 36(12): 633-41 (2011). Binding of TRAIL (tumor necrosis factors [TNF]-related apoptosis-inducing ligand) to death receptors DR4 and DR5 assembles the death-inducing signaling complex (DISC), which induces apoptosis. Tanaka, H. et al., Mol. Cancer Res. 7(4): 557-69 (2009). PRMT5 interacts with DR4 and DR5; knockdown of PRMT5 potentiates TRAIL-induced tumor cell death, indicating that PRMT5 inhibits the killing effect of TRAIL. Id. The molecular mechanism is independent of the PRMT5 methyltransferase activity and involves nuclear factor NF-κB. Id. PRMT5 knockdown sensitizes cancer cells, but not normal cells, to TRAIL-induced death, and PRMT5 overexpression endows a cancer cell line with increased resistance to TRAIL-induced death. Id.

Coexpression of PRMT5 with programmed cell death 4 (PDCD4) influences tumor suppressor properties of PDCD4, resulting in accelerated tumor growth in a murine orthotopic model of breast cancer. (Zakrzewicz, D. et al, “Protein arginine methyltransferases (PRMTs): promising targets for the treatment of pulmonary disorders,” Intl J. Molec. Sci. 13: 12383-400 (2012)). In breast cancer patients whose tumor contain high level of PRMT5, elevated PDCD4 expression correlates with a worse outcome. [Id., citing Powers, M A et al, “Protein arginine methyltransferase 5 accelerates tumor growth by arginine methylation of the tumor suppressor programmed cell death 4,” Cancer Res. 71: 5579-5587 (2011)]. PRMT5 also has been implicated in tumorigenesis by its interaction with p53 protein [Id., citing Jansson, M. et al, “Arginine methylation regulates the p53 response,” Nat. Cell Biol. 10: 1431-39 (2008)], the most frequently inactivated gene in human cancers. [Id., citing Hainaut, P., and Hollstein, M., “p53 and human cancer: the first ten thousand mutations,” Adv. Cancer Res. 77: 81-137 (2000)]. DNA damage induces PRMT5-dependent p53 arginine methylation, an event that changes the biochemical properties and functional outcome of the p53 response, which involves activation of target genes, which are important in regulating p53-dependent growth arrest. [Id., citing Vousden, K H, and Lu, X, “Live or let die: the cell's response to p53,” Nat. Rev. Cancer 2: 594-604 (2002)].

Aberrant methylation and upstream and downstream regulation of epigenetic targets may contribute to a number of diseases, including cancer, metabolic diseases of the pancreas and liver, cardiovascular syndromes (such as endothelial dysfunction and heart failure) and other serious, often progressive diseases. To date, few available agents have shown beneficial modulatory effects against these targets at doses that are both efficacious and safe and with target selectivity that avoids downstream side effects in healthy organs that cause untenable risk: benefit profiles as therapeutics. Moreover, patient heterogeneity has led to a greater understanding of the need for personalized, often polypharmacy, therapy regimens, tailored to a specific profile of disease and patient.

The described invention provides small molecule therapeutics that affect protein methylation via direct inhibition of protein arginine methyltransferases, regulation of nuclear receptor pathways, or a combination thereof. These molecules may exhibit selectivity and optimal PK/PD profiles for the treatment of diseases affected by PRMTs.

### SUMMARY OF THE INVENTION

The described invention provides a method for modulating gene expression of a gene related to proliferation of a population of tumor cells comprising administering a therapeutic amount of a therapeutic compound to a cell, a tissue, or a mammal, wherein the therapeutic amount of the therapeutic compound is effective to modulate a methyltransferase activity of a protein arginine methyltransferase, which in turn modulates methylation of a target protein that affects gene expression of the gene, and may suppress the proliferation of the population of tumor cells. According to one embodiment of the method, the target protein is a histone. According to another embodiment, the histone is one or more of histone H2A, histone H3, or histone H4. According to another embodiment, the protein arginine methyltransferase (PRMT) is one or more of protein arginine methyltransferase 1 (PRMT1), protein arginine methyl transferase 5 (PRMT5), protein arginine methyltransferase 5 complexed with MEP50 (PRMT5/MEK50), or PRMT6. According to another embodiment, modulating the methyltransferase activity of the protein arginine methyltransferase (PRMT) comprises modulating a nuclear receptor or a nuclear receptor signaling pathway. According to another embodiment, modulating the methyltransferase activity of the protein arginine methyltransferase (PRMT) comprises modulating a nuclear receptor or a nuclear receptor signaling pathway by at least 5%, at least 10%, at least 15%, at last 20%, at least 25%, at least 30%, at last 35%, at least 40%, at least 45%, at least 50%, compared to a control. According to another embodiment, the nuclear receptor is one or more of a PPAR nuclear receptor, an FXR nuclear receptor, or an LXR nuclear receptor. According to another embodiment, the nuclear receptor may comprise a nuclear receptor heterodimer complex. According to another embodiment, the PPAR receptor, the FXR nuclear receptor, or the LXR nuclear receptor is complexed with a retinoid X receptor (RXR). According to another embodiment, the therapeutic compound is a PPAR ligand, an FXR ligand, an LXR ligand, or an RXR ligand. According to another embodiment, binding of the therapeutic compound to the PPAR-RXR, FXR-RXR, or LXR-RXR heterodimer complex may modulate binding of the complex to responsive elements in target DNA. According to another embodiment, the therapeutic compound modulates a PPARγ signaling pathway. According to another embodiment, the therapeutic compound is a PPAR agonist that modulates a PPAR nuclear receptor or a PPAR signaling pathway. According to another embodiment, the PPAR agonist is a PPAR-γ agonist selected from Rosiglitazone, a derivative of Rosiglitazone, Pioglitazone or a derivative of Pioglitazone. According to another embodiment, the therapeutic compound is a PPAR-α antagonist that modulates a PPARα nuclear receptor or PPARα signaling pathway. According to another embodiment, the therapeutic compound is an FXR agonist that modulates an FXR nuclear receptor or an FXR signaling pathway. According to another embodiment, the FXR agonist is GW4064, a derivative of GW4064, obeticholic acid, or a derivative of oebeticholic acid. According to another embodiment, the therapeutic compound is an LXR agonist that modulates an LXR receptor or an LXR signaling pathway. According to another embodiment, the LXR agonist is GW3965 or a derivative thereof. According to another embodiment, the therapeutic compound is a TGR5 agonist that modulates a TGR5 signaling pathway. According to another embodiment, the TGR agonist is 3-(2-chlorophenyl)-N-(4-chlorophenyl)-N,5-dimethyl-4-isoxazolecarboxamide (TGR5) or a derivative thereof. According to another embodiment, the method may be effective to reduce tumor growth, and/or to reduce tumor burden in a human cancer. According to another embodiment, the population of tumor cells is a population of leukemia tumor cells, lymphoma tumor cells, sarcoma tumor cells, carcinoma tumor cells, breast tumor cells, lung tumor cells, colorectal tumor cells, enterohepatic tumor cells, bladder tumor cells, mantle cell tumor cells, pancreatic tumor cells, prostate tumor cells, thyroid tumor cells, or brain tumor cells. According to another embodiment, the therapeutic amount of the therapeutic compound may be effective to induce apoptosis in the population of tumor cells.

### BRIEF DESCRIPTION OF THE DRAWINGS

FIG. 1 shows the three main forms of methylarginine in eukaryotes (taken from McBride, A. E. and Silver, P. A., “State of the Arg: Protein Methylation at Arginine Comes of Age,” Cell 106: 5-8 (2001).

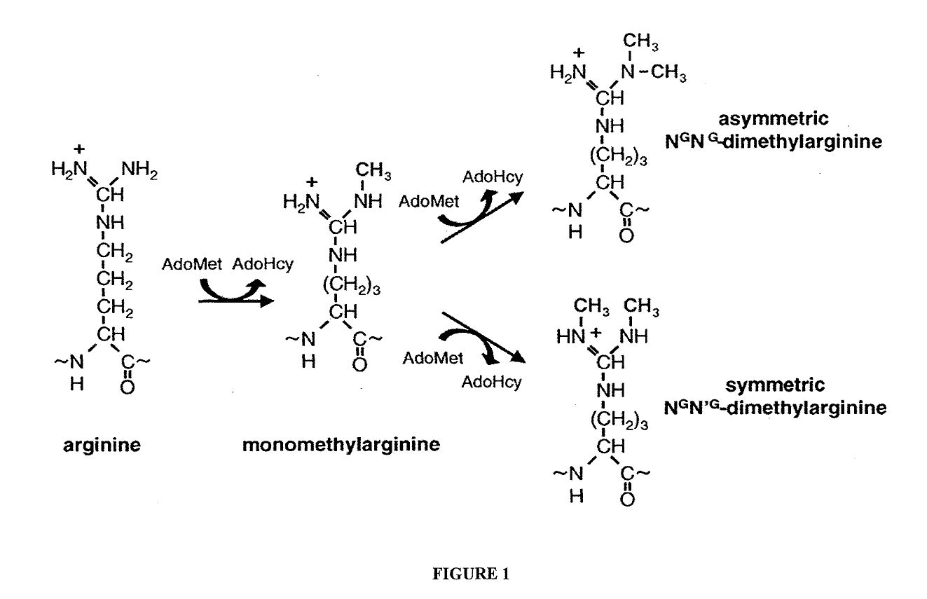

FIG. 2 is an overview of the mechanisms of bile acid synthesis repression by FXR. After binding any bile acids, FXR induces the expression of SHP, which in turn interacts with LRH-1 or HNF4α to decrease the transcription of CYP7A1 and CYP8B1, respectively. Simultaneously FXR induces the expression of FGF-19. FGF-19 interacts with its cognate receptor FGFR-4 to negatively regulate bile acid production by repressing YP7A1 and CYP8B1 gene expression by interfering with the JNK pathway. (From Claudel, T. et al, “The Farnesoid X Receptor, A Molecular Lin k Between Bile Acid and Lipid and Glucose Metabolism,” Arterioscler. Thromb. Vasc. Biol. 25: 2020-31 (2005)).

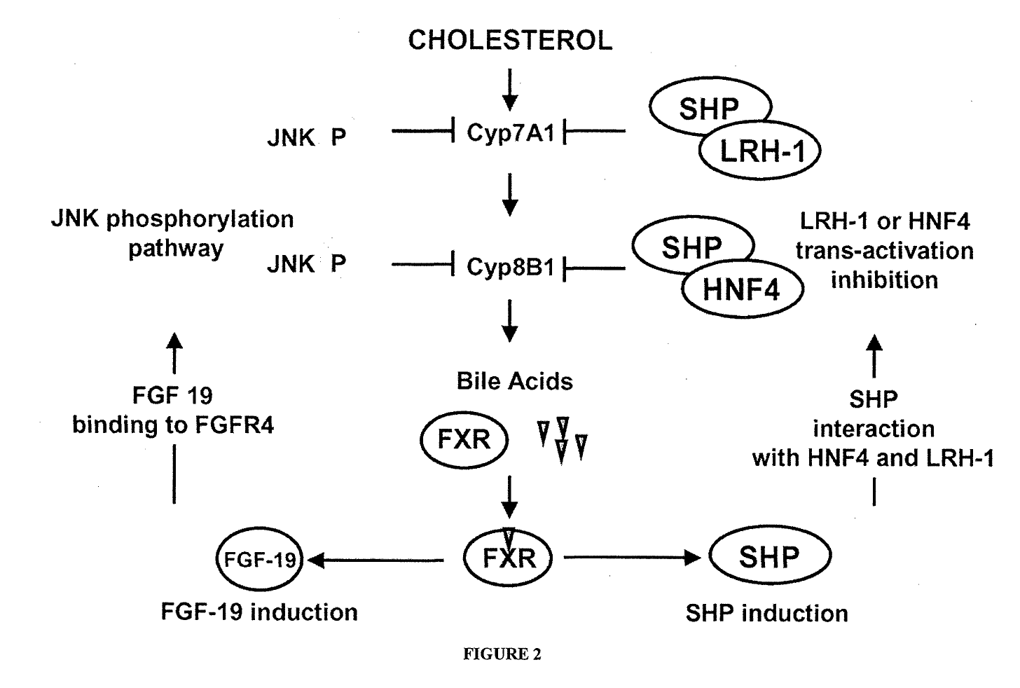

FIG. 3 shows an IC50 curve depicting inhibition of PRMT1 by S-(5′-Adenosyl)-L-homocysteine (SAH).

FIG. 4 shows an IC50 curve depicting inhibition of PRMT5 by S-(5′-Adenosyl)-L-homocysteine (SAH).

FIG. 5 shows an IC50 curve depicting inhibition of PRMT5/MEP50 by S-(5′-Adenosyl)-L-homocysteine (SAH).

FIG. 6 shows an IC50 curve depicting inhibition of PRMT6 by S-(5′-Adenosyl)-L-homocysteine (SAH).

FIG. 7 shows an IC50 curve depicting inhibition of PRMT5/MEP50 by compound GW 4064 (black squares) and GW 3965 (white triangles).

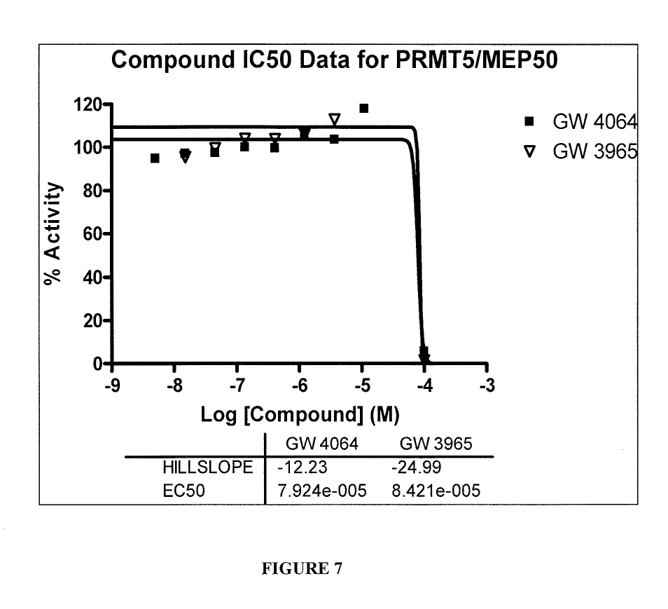

FIG. 8 shows an IC50 curve depicting inhibition of PRMT5/MEP50 by S-(5′-Adenosyl)-L-homocysteine (SAH).

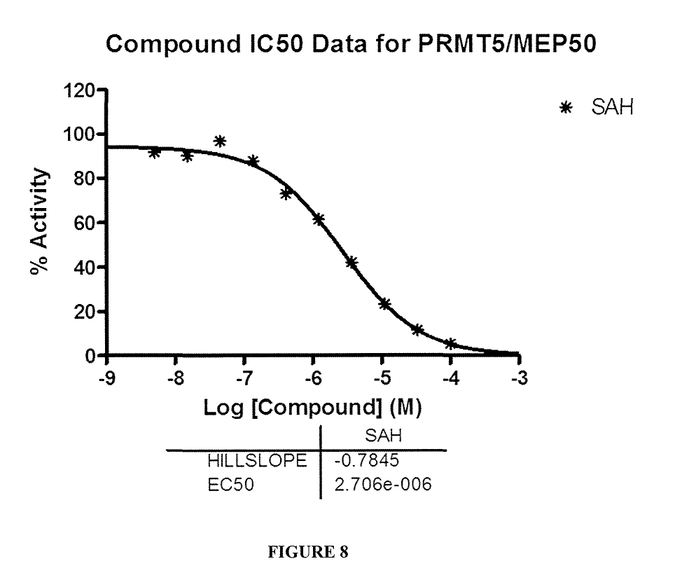

### DETAILED DESCRIPTION

### Glossary

The term “activator” as used herein refers to proteins that bind to genes known as enhancers, which help determine which genes are switched on and speed up transcription.

The term “agonist” as used herein refers to a chemical substance capable of activating a receptor to induce a full or partial pharmacological response. Receptors can be activated or inactivated by either endogenous or exogenous agonists and antagonists, resulting in stimulating or inhibiting a biological response. A physiological agonist is a substance that creates the same bodily responses, but does not bind to the same receptor. An endogenous agonist for a particular receptor is a compound naturally produced by the body which binds to and activates that receptor. A superagonist is a compound that is capable of producing a greater maximal response than the endogenous agonist for the target receptor, and thus an efficiency greater than 100%. This does not necessarily mean that it is more potent than the endogenous agonist, but is rather a comparison of the maximum possible response that can be produced inside a cell following receptor binding. Full agonists bind and activate a receptor, displaying full efficacy at that receptor. Partial agonists also bind and activate a given receptor, but have only partial efficacy at the receptor relative to a full agonist. An inverse agonist is an agent which binds to the same receptor binding-site as an agonist for that receptor and reverses constitutive activity of receptors. Inverse agonists exert the opposite pharmacological effect of a receptor agonist. An irreversible agonist is a type of agonist that binds permanently to a receptor in such a manner that the receptor is permanently activated. It is distinct from a mere agonist in that the association of an agonist to a receptor is reversible, whereas the binding of an irreversible agonist to a receptor is believed to be irreversible. This causes the compound to produce a brief burst of agonist activity, followed by desensitization and internalization of the receptor, which with long-term treatment produces an effect more like an antagonist. A selective agonist is specific for one certain type of receptor.

The term “allosteric modulation” as used herein refers to the process of modulating a receptor by the binding of allosteric modulators at a different site (i.e., regulatory site) other than of the endogenous ligand (orthosteric ligand) of the receptor and enhancing or inhibiting the effects of the endogenous ligand. It normally acts by causing a conformational change in a receptor molecule, which results in a change in the binding affinity of the ligand. Thus, an allosteric ligand “modulates” its activation by a primary “ligand” and can adjust the intensity of the receptor's activation. Many allosteric enzymes are regulated by their substrate, such a substrate is considered a “homotropic allosteric modulator.” Non-substrate regulatory molecules are called “heterotropic allosteric modulators.”

The term “allosteric regulation” is the regulation of an enzyme or other protein by binding an effector molecule at the proteins allosteric site (meaning a site other than the protein's active site). Effectors that enhance the protein's activity are referred to as “allosteric activators”, whereas those that decrease the protein's activity are called “allosteric inhibitors.” Thus, “allosteric activation” occurs when the binding of one ligand enhances the attraction between substrate molecules and other binding sites; “allosteric inhibition” occurs when the binding of one ligand decrease the affinity for substrate at other active sites. The term “antagonist” as used herein refers to a substance that counteracts the effects of another substance.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligomerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits TAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “biomarkers” (or “biosignatures”) as used herein refers to peptides, proteins, nucleic acids, antibodies, genes, metabolites, or any other substances used as indicators of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

The term “bound” or any of its grammatical forms as used herein refers to the capacity to hold onto, attract, interact with or combine with.

The term “blocker” as used herein refers to a substance that inhibits the physiological action of another substance.

The term “cDNA” refers to DNA synthesized from a mature mRNA template. cDNA most often is synthesized from mature mRNA using the enzyme reverse transcriptase. The enzyme operates on a single strand of mRNA, generating its complementary DNA based on the pairing of RNA base pairs (A, U, G, C) to their DNA complements (T, A, C, G). There are several methods known for generating cDNA to obtain, for example, eukaryotic cDNA whose introns have been spliced. Generally, these methods incorporate the following steps: a) a eukaryotic cell transcribes the DNA (from genes) into RNA (pre-mRNA); b) the same cell processes the pre-mRNA strands by splicing out introns, and adding a poly-A tail and 5′ Methyl-Guanine cap; c) this mixture of mature mRNA strands are extracted from the cell; d) a poly-T oligonucleotide primer is hybridized onto the poly-A tail of the mature mRNA template (reverse transcriptase requires this double-stranded segment as a primer to start its operation); e) reverse transcriptase is added, along with deoxynucleotide triphosphates (A, T, G, C); f) the reverse transcriptase scans the mature mRNA and synthesizes a sequence of DNA that complements the mRNA template. This strand of DNA is complementary DNA. (see also Current Protocols in Molecular Biology, John Wiley & Sons, incorporated in its entirety herein).

The term “contact” and its various grammatical forms as used herein refers to a state or condition of touching or of immediate or local proximity. Contacting a composition to a target destination, such as, but not limited to, an organ, a tissue, a cell, or a tumor, may occur by any means of administration known to the skilled artisan.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines.

The term “derivative” as used herein means a compound that may be produced from another compound of similar structure in one or more steps. A “derivative” or “derivatives” of a peptide or a compound retains at least a degree of the desired function of the peptide or compound. Accordingly, an alternate term for “derivative” may be “functional derivative.” Derivatives can include chemical modifications of the peptide, such as alkylation, acylation, carbamylation, iodination or any modification that derivatizes the peptide. Such derivatized molecules include, for example, those molecules in which free amino groups have been derivatized to form amine hydrochlorides, p-toluene sulfonyl groups, carbobenzoxy groups, t-butyloxycarbonyl groups, chloroacetyl groups or formal groups. Free carboxyl groups can be derivatized to form salts, esters, amides, or hydrazides. Free hydroxyl groups can be derivatized to form O-acyl or O-alkyl derivatives. The imidazole nitrogen of histidine can be derivatized to form N-im-benzylhistidine. Also included as derivatives or analogues are those peptides that contain one or more naturally occurring amino acid derivative of the twenty standard amino acids, for example, 4-hydroxyproline, 5-hydroxylysine, 3-methylhistidine, homoserine, ornithine or carboxyglutamate, and can include amino acids that are not linked by peptide bonds. Such peptide derivatives can be incorporated during synthesis of a peptide, or a peptide can be modified by well-known chemical modification methods (see, e.g., Glazer et al., Chemical Modification of Proteins, Selected Methods and Analytical Procedures, Elsevier Biomedical Press, New York (1975)).

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like. When a nucleic acid is prepared or altered synthetically, advantage can be taken of known codon preferences of the intended host where the nucleic acid is to be expressed.

The term “detectable response” refers to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “domain” as used herein refers to a region of a protein with a characteristic primary structure and function.

The term “effector” as used herein refers to a molecule that binds to a protein and thereby alters the activity of that protein.

The term “epigenetic changes” as used herein refers to changes inherited during mitosis/meiosis without altering the underlying DNA sequences.

The term “expression system” refers to a genetic sequence, which includes a protein encoding region operably linked to all of the genetic signals necessary to achieve expression of the protein encoding region. Traditionally, the expression system will include a regulatory element such as, for example, a promoter or enhancer, to increase transcription and/or translation of the protein encoding region, or to provide control over expression. The regulatory element may be located upstream or downstream of the protein encoding region, or may be located at an intron (non-coding portion) interrupting the protein encoding region. Alternatively, it also is possible for the sequence of the protein encoding region itself to comprise regulatory ability.

The term “functional equivalent” or “functionally equivalent” are used interchangeably herein to refer to substances, molecules, polynucleotides, proteins, peptides, or polypeptides having similar or identical biological activity to a reference substance, molecule, polynucleotide, protein, peptide, or polypeptide. Any polypeptide that retains the biological activity of the PRMT protein may be used as such a functional equivalent. Such functional equivalents include those wherein one or more amino acids are substituted, deleted, added, or inserted to the natural occurring amino acid sequence of the PRMT. Alternatively the polypeptide may be composed of an amino acid sequence having sequence identity to the sequence of the respective PRMT. The polypeptide can be encoded by a polynucleotide that hybridizes under stringent conditions to the naturally occurring nucleotide sequence of the PRMT gene.

The term “GAR motif” as used herein refers to glycine- and arginine-rich patches in a protein.

The term “gene” as used herein, refers to the entire DNA sequence, including exons, introns and noncoding transcription control regions, necessary for production of a functional protein or RNA.

The term “gene expression” as used herein refers to the overall process by which the information encoded in a gene is converted into an observable phenotype, most commonly production of a protein.

The term “half maximal inhibitory concentration” (“IC50”) is a measure of the effectiveness of a compound in inhibiting a biological or biochemical function.

The term “homeodomain” as used herein refers to a conserved DNA binding motif found in many developmentally important transcription factors.

The term “hybridization” refers to the process of combining complementary, single-stranded nucleic acids into a single molecule. Nucleotides will bind to their complement under normal conditions, so two perfectly complementary strands will bind (or ‘anneal’) to each other readily. However, due to the different molecular geometries of the nucleotides, a single inconsistency between the two strands will make binding between them more energetically unfavorable. Measuring the effects of base incompatibility by quantifying the rate at which two strands anneal can provide information as to the similarity in base sequence between the two strands being annealed. The term “specifically hybridizes” as used herein refers to the process whereby a nucleic acid distinctively or definitively forming base pairs with complementary regions of at least one strand of DNA that was not originally paired to the nucleic acid. For example, a nucleic acid that may bind or hybridize to at least a portion of an mRNA of a cell encoding a peptide comprising a PRMT sequence may be considered a nucleic acid that specifically hybridizes. A nucleic acid that selectively hybridizes undergoes hybridization, under stringent hybridization conditions, of the nucleic acid sequence to a specified nucleic acid target sequence to a detectably greater degree (e.g., at least 2-fold over background) than its hybridization to non-target nucleic acid sequences and to the substantial exclusion of non-target nucleic acids. Selectively hybridizing sequences typically have about at least 80% sequence identity, at least 90% sequence identity, or at least 100% sequence identity (i.e., complementary) with each other.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%.

The term “inhibitor” as used herein refers to a molecule that binds to an enzyme thereby decreasing enzyme activity. Enzyme inhibitors are molecules that bind to enzymes thereby decreasing enzyme activity. The binding of an inhibitor may stop substrate from entering the active site of the enzyme and/or hinder the enzyme from catalyzing its reaction. Inhibitor binding is either reversible or irreversible. Irreversible inhibitors usually react with the enzyme and change it chemically, for example, by modifying key amino acid residues needed for enzymatic activity. In contrast, reversible inhibitors bind non-covalently and produce different types of inhibition depending on whether these inhibitors bind the enzyme, the enzyme-substrate complex, or both. Enzyme inhibitors often are evaluated by their specificity and potency.

The term “intron” or “intervening sequence” as used herein refers to part of a primary transcript (or the DNA encoding it) that is removed by splicing during RNA processing and is not included in the mature, functional mRNA, rRNA or tRNA.

The term “isolated” is used herein to refer to material, such as, but not limited to, a nucleic acid, peptide, polypeptide, or protein, which is: (1) substantially or essentially free from components that normally accompany or interact with it as found in its naturally occurring environment. The terms “substantially free” or “essentially free” are used herein to refer to considerably or significantly free of, or more than about 95% free of, or more than about 99% free of. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment. The alteration to yield the synthetic material may be performed on the material within, or removed, from its natural state. For example, a naturally occurring nucleic acid becomes an isolated nucleic acid if it is altered, or if it is transcribed from DNA that has been altered, by means of human intervention performed within the cell from which it originates. See, for example, Compounds and Methods for Site Directed Mutagenesis in Eukaryotic Cells, Kmiec, U.S. Pat. No. 5,565,350; In Vivo Homologous Sequence Targeting in Eukaryotic Cells; Zarling et al., PCT/US93/03868. Likewise, a naturally occurring nucleic acid (for example, a promoter) becomes isolated if it is introduced by non-naturally occurring means to a locus of the genome not native to that nucleic acid. Nucleic acids that are “isolated” as defined herein also are referred to as “heterologous” nucleic acids.

The compositions described herein are isolated molecules. An “isolated molecule” is a molecule that is substantially pure and is free of other substances with which it is ordinarily found in nature or in vivo systems to an extent practical and appropriate for its intended use. In particular, the compositions are sufficiently pure and are sufficiently free from other biological constituents of host cells so as to be useful in, for example, producing pharmaceutical preparations or sequencing if the composition is a nucleic acid, peptide, or polysaccharide. Because compositions may be admixed with a pharmaceutically-acceptable carrier in a pharmaceutical preparation, the compositions may comprise only a small percentage by weight of the preparation. The composition is nonetheless substantially pure in that it has been substantially separated from the substances with which it may be associated in living systems or during synthesis. As used herein, the term “substantially pure” refers purity of at least 75%, at least 80%, at least 85%, at least 90%, at least 95% or at least 99% pure as determined by an analytical protocol. Such protocols may include, for example, but are not limited to, FACS, HPLC, gel electrophoresis, chromatography, and the like.

The term “isoform” refers to a version of a protein that has the same function as another protein but that has some small difference(s) in its sequence.

The term “kinase” as used herein refers to a type of enzyme that transfers phosphate groups from high-energy donor molecules to specific target molecules or substrates. High-energy donor groups may include, but are not limited, to ATP.

The term “kinase activity” as used herein refers to kinase mediated phosphorylation of a kinase substrate.

The term “kinase substrate” as used herein refers to a substrate that can be phosphorylated by a kinase. The term “knockout” as used herein refers to a technique for selectively inactivating a gene by replacing it with a mutant allele in an otherwise normal organism.

The term “ligand” as used herein refers to a molecule that binds to another; for example an antibody, hormone or drug that binds to a receptor.

The term “mimetic” refers to chemicals containing chemical moieties that mimic the function of a peptide. For example, if a peptide contains two charged chemical moieties having functional activity, mimetic places two charged chemical moieties in a spatial orientation and constrained structure so that the charged chemical function is maintained in three-dimensional space.

The term “modulate” as used herein means to regulate, alter, adapt, or adjust to a certain measure or proportion. Such modulation may be any change, including an undetectable change.

The term “mRNA” as used herein refers to the RNA product produced by transcription of DNA by RNA polymerase. In eukaryotes, the initial RNA product (primary transcript) undergoes processing to yield functional mRNA which is transported to the cytoplasm for its translation into protein.

The term “nucleosome” as used herein refers to a small structural unit of chromatin consisting of a disk-shaped core of histone proteins around which a segment of DNA is wrapped.

The term “nucleic acid” is used herein to refer to a deoxyribonucleotide or ribonucleotide polymer in either single- or double-stranded form, and unless otherwise limited, encompasses known analogues having the essential nature of natural nucleotides in that they hybridize to single-stranded nucleic acids in a manner similar to naturally occurring nucleotides (e.g., peptide nucleic acids).

The term “nucleotide” is used herein to refer to a chemical compound that consists of a heterocyclic base, a sugar, and one or more phosphate groups. In the most common nucleotides, the base is a derivative of purine or pyrimidine, and the sugar is the pentose deoxyribose or ribose. Nucleotides are the monomers of nucleic acids, with three or more bonding together in order to form a nucleic acid. Nucleotides are the structural units of RNA, DNA, and several cofactors, including, but not limited to, CoA, FAD, DMN, NAD, and NADP. Purines include adenine (A), and guanine (G); pyrimidines include cytosine (C), thymine (T), and uracil (U).

The phrase “operably linked” as used herein refers to a first sequence(s) or domain being positioned sufficiently proximal to a second sequence(s) or domain so that the first sequence(s) or domain can exert influence over the second sequence(s) or domain or a region under control of that second sequence or domain.

The term “polynucleotide” refers to a deoxyribopolynucleotide, ribopolynucleotide, or analogues thereof that have the essential nature of a natural ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide may be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine, or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including among other things, simple and complex cells.

The term “protein” is used herein to refer to a large complex molecule or polypeptide composed of amino acids. The sequence of the amino acids in the protein is determined by the sequence of the bases in the nucleic acid sequence that encodes it.

The terms “peptide”, “polypeptide” and “protein” also apply to amino acid polymers in which one or more amino acid residue is an artificial chemical analogue of a corresponding naturally occurring amino acid, as well as to naturally occurring amino acid polymers. The essential nature of such analogues of naturally occurring amino acids is that, when incorporated into a protein that protein is specifically reactive to antibodies elicited to the same protein but consisting entirely of naturally occurring amino acids. The terms “polypeptide”, “peptide” and “protein” also are inclusive of modifications including, but not limited to, glycosylation, lipid attachment, sulfation, gamma-carboxylation of glutamic acid residues, hydroxylation and ADP-ribosylation. It will be appreciated, as is well known and as noted above, that polypeptides may not be entirely linear. For instance, polypeptides may be branched as a result of ubiquitination, and they may be circular, with or without branching, generally as a result of posttranslational events, including natural processing event and events brought about by human manipulation which do not occur naturally. Circular, branched and branched circular polypeptides may be synthesized by non-translation natural process and by entirely synthetic methods, as well.

The term “peptidomimetic” refers to a small protein-like chain designed to mimic or imitate a peptide. A peptidomimetic may comprise non-peptidic structural elements capable of mimicking (meaning imitating) or antagonizing (meaning neutralizing or counteracting) the biological action(s) of a natural parent peptide.

The term “primary transcript” as used herein refers to the initial RNA product containing introns and exons produced by transcription of DNA.

The term “promoter” refers to a region of DNA upstream, downstream, or distal, from the start of transcription and involved in recognition and binding of RNA polymerase and other proteins to initiate transcription. For example, T7, T3 and Sp6 are RNA polymerase promoter sequences. In RNA synthesis, promoters are a means to demarcate which genes should be used for messenger RNA creation and by extension, control which proteins the cell manufactures. Promoters represent critical elements that can work in concert with other regulatory regions (enhancers, silencers, boundary elements/insulators) to direct the level of transcription of a given gene.

The term “proteasome” as used herein refers to a large multifunctional protease complex in the cytosol that degrades intracellular proteins marked for destruction by attachment of multiple ubiquitin molecules.

The term “repressor” as used herein refers to proteins that bind to genes called “silencers”, which interfere with activator proteins and slow down transcription.

The term “regulatory sequence” (also referred to as a “regulatory region” or “regulatory element”) refers to a promoter, enhancer or other segment of DNA where regulatory proteins, such as transcription factors, bind preferentially to control gene expression and thus protein expression.

The terms “residue” or “amino acid residue” or “amino acid” are used interchangeably to refer to an amino acid that is incorporated into a protein, a polypeptide, or a peptide, including, but not limited to, a naturally occurring amino acid and known analogues of natural amino acids that can function in a similar manner as naturally occurring amino acids.

Sequences:

The following terms are used herein to describe the sequence relationships between two or more nucleic acids or polynucleotides: (a) “reference sequence”, (b) “comparison window”, (c) “sequence identity”, (d) “percentage of sequence identity”, and (e) “substantial identity”.

The term “reference sequence” refers to a sequence used as a basis for sequence comparison. A reference sequence may be a subset or the entirety of a specified sequence; for example, as a segment of a full-length cDNA or gene sequence, or the complete cDNA or gene sequence.

The term “comparison window” refers to a contiguous and specified segment of a polynucleotide sequence, wherein the polynucleotide sequence may be compared to a reference sequence and wherein the portion of the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be at least 30 contiguous nucleotides in length, at least 40 contiguous nucleotides in length, at least 50 contiguous nucleotides in length, at least 100 contiguous nucleotides in length, or longer. Those of skill in the art understand that to avoid a high similarity to a reference sequence due to inclusion of gaps in the polynucleotide sequence, a gap penalty typically is introduced and is subtracted from the number of matches.

Methods of alignment of sequences for comparison are well-known in the art. Optimal alignment of sequences for comparison may be conducted by the local homology algorithm of Smith and Waterman, Adv. Appl. Math. 2:482 (1981); by the homology alignment algorithm of Needleman and Wunsch, J. Mol. Biol. 48:443 (1970); by the search for similarity method of Pearson and Lipman, Proc. Natl. Acad. Sci. 85:2444 (1988); by computerized implementations of these algorithms, including, but not limited to: CLUSTAL in the PC/Gene program by Intelligenetics, Mountain View, Calif.; GAP, BESTFIT, BLAST, FASTA, and TFASTA in the Wisconsin Genetics Software Package, Genetics Computer Group (GCG), 575 Science Dr., Madison, Wis., USA; the CLUSTAL program is well described by Higgins and Sharp, Gene 73:237-244 (1988); Higgins and Sharp, CABIOS 5:151-153 (1989); Corpet, et al., Nucleic Acids Research 16:10881-90 (1988); Huang, et al., Computer Applications in the Biosciences 8:155-65 (1992), and Pearson, et al., Methods in Molecular Biology 24:307-331 (1994). The BLAST family of programs, which can be used for database similarity searches, includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York (1995).

Unless otherwise stated, sequence identity/similarity values provided herein refer to the value obtained using the BLAST 2.0 suite of programs using default parameters. Altschul et al., Nucleic Acids Res. 25:3389-3402 (1997). Software for performing BLAST analyses is publicly available, e.g., through the National Center for Biotechnology-Information (http://www.hcbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits then are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, a cutoff of 100, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. BLAST searches assume that proteins may be modeled as random sequences. However, many real proteins comprise regions of nonrandom sequences which may be homopolymeric tracts, short-period repeats, or regions enriched in one or more amino acids. Such low-complexity regions may be aligned between unrelated proteins even though other regions of the protein are entirely dissimilar. A number of low-complexity filter programs may be employed to reduce such low-complexity alignments. For example, the SEG (Wooten and Federhen, Comput. Chem., 17:149-163 (1993)) and XNU (Claverie and States, Comput. Chem., 17:191-201 (1993)) low-complexity filters may be employed alone or in combination.

The term “sequence identity” or “identity” in the context of two nucleic acid or polypeptide sequences is used herein to refer to the residues in the two sequences that are the same when aligned for maximum correspondence over a specified comparison window. When percentage of sequence identity is used in reference to proteins it is recognized that residue positions that are not identical often differ by conservative amino acid substitutions, i.e., where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, Computer Applic. Biol. Sci., 4:11-17 (1988) e.g., as implemented in the program PC/GENE (Intelligenetics, Mountain View, Calif., USA).

The term “percentage of sequence identity” is used herein mean the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, at least 80% sequence identity, at least 90% sequence identity and at least 95% sequence identity, compared to a reference sequence using one of the alignment programs described using standard parameters. One of skill will recognize that these values may be adjusted appropriately to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or at least 70%, at least 80%, at least 90%, or at least 95%. Another indication that nucleotide sequences are substantially identical is if two molecules hybridize to each other under stringent conditions. However, nucleic acids that do not hybridize to each other under stringent conditions are still substantially identical if the polypeptides that they encode are substantially identical. This may occur, e.g., when a copy of a nucleic acid is created using the maximum codon degeneracy permitted by the genetic code. One indication that two nucleic acid sequences are substantially identical is that the polypeptide that the first nucleic acid encodes is immunologically cross reactive with the polypeptide encoded by the second nucleic acid.

The terms “substantial identity” in the context of a peptide indicates that a peptide comprises a sequence with at least 70% sequence identity to a reference sequence, at least 80%, at least 85%, at least 90% or 95% sequence identity to the reference sequence over a specified comparison window. Optionally, optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides which are “substantially similar” share sequences as noted above except that residue positions that are not identical may differ by conservative amino acid changes.

The term “SH3 domain” as used herein refers to a small protein module containing approximately 50 amino acid residues, which has a characteristic fold which consists of five or six beta-strands arranged as two tightly packed anti-parallel beta sheets. The linker regions may contain short helices. The surface of the SH3-domain bears a flat, hydrophobic ligand-binding pocket which consists of three shallow grooves defined by conservative aromatic residues in which the ligand adopts an extended left-handed helical arrangement. The ligand binds with low affinity but this may be enhanced by multiple interactions. The region bound by the SH3 domain is in all cases proline-rich and contains PXXP as a core-conserved binding motif. SH3 domains bind to target proteins through sequences containing proline and hydrophobic amino acids. Pro-containing polypeptides may bind to SH3 domains in 2 different binding orientations. The function of the SH3 domain is not well understood; it is believed that they may mediate many diverse processes such as increasing local concentration of proteins, altering their subcellular location and mediating the assembly of large multiprotein complexes ((Morton, C J, Campbell I D, “SH3 domains. Molecular ‘Velcro’”; Curr. Biol. 4(7): 615-17 (1994)).

The term “spliceosome” as used herein refers to the macromolecular complex responsible for removing intron sequences that interrupt many eukaryotic gene transcripts. The spliceosome is composed of five small nuclear ribonucleoproteins (snRNPs), known as U1, U2, U3, U4, U5 and U6, and more than 100 additional proteins.

The term “STATS” or signal transducers and activators of transcription, as used herein refers to a family of latent cytoplasmic proteins that are activated to participate in gene control when cells encounter various extracellular polypeptides.

The term “substitution” is used herein to refer to that in which a base or bases are exchanged for another base or bases in DNA. Substitutions may be synonymous substitutions or nonsynonymous substitutions. As used herein, “synonymous substitutions” refer to substitutions of one base for another in an exon of a gene coding for a protein, such that the amino acid sequence produced is not modified. The term “nonsynonymous substitutions” as used herein refer to substitutions of one base for another in an exon of a gene coding for a protein, such that the amino acid sequence produced is modified.

The term “substrate” as used herein refers to a molecule that undergoes a change in a reaction catalyzed by an enzyme.

The term “transcription activation” as used herein refers to the output of the interaction between the sequence-specific activator and basal transcription machinery, which increases the efficiency and/or stability of the transcription machinery complex.

The term “transcription co-activator” as used herein refers to molecules that coordinate signals from activator and repressor proteins. A transcription co-activator does not bind DNA itself.

The term “transcription factor” as used herein refers to any protein, other than RNA polymerase, required to initiate or regulate transcription in eukaryotic cells. General factors, required for transcription of all genes, participate in formation of the transcription-initiation complex near the start site. Specific factors stimulate (or repress) transcription of particular genes by binding to their regulatory sequences.

The terms “variants”, “mutants”, and “derivatives” are used herein to refer to nucleotide sequences with substantial identity to a reference nucleotide sequence. The differences in the sequences may by the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence.

A skilled artisan likewise can produce polypeptide variants having single or multiple amino acid substitutions, deletions, additions or replacements. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, such as, for example, an epitope for an antibody. The techniques for obtaining such variants, including genetic (suppressions, deletions, mutations, etc.), chemical, and enzymatic techniques are known to the skilled artisan. As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “WW domains” as used herein refers to protein modules that mediate protein-protein interactions through recognition of proline-rich peptide motifs and phosphorylated serine/threonine-proline sites. Ingham, R J et al, “WW domains provide a platform for the assembly of multiprotein networks,” Mol. Cell Biol. 25(16): 7092-106 (2005))

The term “zinc finger” as used herein refers to a conserved DNA-binding motif composed of protein domains folded around a zinc ion. Zinc fingers are present in several types of eukaryotic transcription factors.

According to one aspect, the described invention provides a method for modulating gene expression of a gene related to proliferation of a population of tumor cells. According to some embodiments, gene expression of the gene is modulated by providing a therapeutic compound that is effective to suppress methyltransferase activity of a methyltransferase, which modulates methylation of a target protein that affects gene expression of the gene. According to some embodiments, the target protein is a histone. According to some embodiments, the histone is one or more of histone H2A, histone H3, or histone H4.

According to some embodiments, the therapeutic compound is effective to modulate protein methylation of the target protein by modulating activity of one or more protein arginine methyltransferases. According to some embodiments, the protein arginine methyltransferase is PRMT1. According to some embodiments, the protein arginine methyltransferase is PRMT2. According to some embodiments, the protein arginine methyltransferase is PRMT3. According to some embodiments, the protein arginine methyltransferase is PRMT4/CARN1. According to some embodiments, the protein arginine methyltransferase is PRMT5. According to some embodiments, the protein arginine methyltransferase is PRMT5 in complex with MEP50. According to some embodiments, the protein arginine methyltransferase is PRMT6. According to some embodiments, the protein arginine methyltransferase is PRMT7. According to some embodiments, the protein arginine methyltransferase is PRMT8. According to some embodiments, the protein arginine methyltransferase is PRMT9.

According to some embodiments, the therapeutic compound is effective to suppress the methyltransferase activity of the protein arginine methyltransferase. According to some embodiments the therapeutic compound is effective to suppress methyltransferase activity of the protein arginine methyltransferase by at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%.

According to some embodiments, the therapeutic compound is effective to suppress methyltransferase activity of the protein arginine methyltransferase by modulating a nuclear receptor or a nuclear receptor signaling pathway.

According to some embodiments, the methyltransferase activity of the PRMT is negatively modulated (i.e., inhibited) by the nuclear receptor. According to some embodiments, the nuclear receptor is one or more of a PPAR nuclear receptor, an FXR nuclear receptor, or an LXR nuclear receptor. According to some embodiments, the PPAR nuclear receptor is a PPARα receptor, a PPARδ receptor or a PPARγ receptor. According to some embodiments the nuclear receptor is a nuclear receptor heterodimer complex, e.g., the PPAR receptor, the FXR nuclear receptor, or the LXR nuclear receptor is complexed with a retinoid X receptor (RXR). According to some embodiments, the therapeutic compound is a PPAR ligand, an FXR ligand, an LXR ligand, or an RXR ligand. According to some embodiments, binding of the therapeutic compound to the PPAR-RXR, FXR-RXR, or LXR-RXR heterodimer complex modulates binding of the complex to responsive elements in target DNA.

According to some embodiments, the therapeutic compound is effective to suppress methyltransferase activity of the protein arginine methyltransferase by modulating a nuclear receptor signaling pathway. According to some embodiments, the therapeutic compound modulates a PPARγ signaling pathway by, e.g., at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%. A therapeutic compound as disclosed herein may be capable of binding to all isoforms of PPAR-γ, or may be capable of selectively binding to either PPAR-γ1, PPAR-γ2, PPAR-γ3, or any combination thereof.

According to some embodiments, the therapeutic compound may be a PPAR agonist or derivative thereof. Examples of PPARγ agonists include, without limitation, Benzbromarone, a cannabidiol, Cilostazol, Curcumin, Delta(9)-tetrahydrocannabinol, glycyrrhetinic acid, Indomethacin, Irbesartan, Monascin, mycophenolic acid, Resveratrol, 6-shogaol, Telmisartan, a thiazolidinedione, (e.g., Rosiglitazone, Pioglitazone, and Troglitazone), a NSAID, and a fibrate. Other PPAR agonists, for example, 2-(4-((2-methoxy-6-phenylpyridin-3-yl)methoxy)phenoxy)-2-methyl-propanoic acid; 2-(4-((2-methoxy-6-phenylpyridin-3-yl)methoxy)phenoxy)ethanoic acid; 2-(4-((2-methoxy-6-phenylpyridin-3-yl)methoxy)phenoxy)propanoic acid; 2-(4-(((2-methoxy-6-phenylpyridin-3yl)methyl)amino)phenoxy)ethanoic acid; 2-(4-(((2-methoxy-6-phenylpyridin-3yl)methyl)amino)phenoxy)propanoic acid; 2-(4-((2-tert-butyloxy-6-phenylpyridin-3-yl)methoxy)phenoxy)-2-methyl-propanoic acid; 2-(4-(((2-tert-butyloxy-6-phenylpyridin-3-yl)methoxy)phenoxy)ethanoic acid; 2-(4-((2-tert-butyloxy-6-phenylpyridin-3-yl)methyl)amino)phenoxy)ethanoic acid; 2-(4-(((2-tert-butyloxy-6-phenylpyridin-3-yl)methyl)amino)phenoxy)-2-methyl-propanoic acid; 2-(4-(((2-tert-butyloxy-6-phenylpyridin-3-yl)methyl)amino) phenoxy)-propanoic acid; 2-(4-(((2-methoxy-6-phenylpyridin-3-yl)methyl)amino)phenylthio)-2-methyl-propanoic acid; 2-(4-(((2-methoxy-6-phenylpyridin-3-yl)methyl)amino)phenoxy)-2-methyl-propanoic acid; 2-(3-(((2-methoxy-6-phenylpyridin-3-yl)methyl)amino)phenoxy)-2-methyl-propanoic acid; 2-(3-(((2-methoxy-6-phenylpyridin-3-yl)methyl)amino)phenoxy)ethanoic acid; 2-(4-((2-hexyloxy-6-phenylpyridin-3-yl)methoxy)phenoxy)ethanoic acid; 2-(4-(((2-methoxy-6-phenylpyridin-3-yl)methyl)amino)phenylthio)-ethanoic acid; 2-(4-((2-hexyloxy-6-phenylpyridin-3-yl)methoxy)phenoxy)-2-methyl-propanoic acid; 2-(4-((2-cyclohexyloxy-6-phenylpyridin-3-yl)methoxy)phenoxy)ethanoic acid; 3-(4-(((2-methoxy-6-phenylpyridin-3-yl)methyl)amino)phenyl)propanoic acid; 2-(4-((6-phenyl-2-(piperidin-1-yl)pyridin-3-yl)methoxy)phenoxy)-ethanoic acid; 2-(4-((2-methoxy-6-(4-(trifluoromethyl)phenyl)pyridin-3-yl)methoxy)-phenoxy)-2-methylpropanoic acid; 2-(4-(((2-methoxy-6-(4-(trifluoromethyl)phenyl)pyridin-3-yl)methyl)amino)-phenylthio)-2-methylpropanoic acid; 2-(4-(((2-methoxy-6-(4-(trifluoromethyl)phenyl)pyridin-3-yl)methyl)amino)-phenylthio)ethanoic acid; 2-(4-((2-methoxy-6-(4-(trifluoromethyl)phenyl)pyridin-3-yl)methoxy)-phenoxy)ethanoic acid; 2-(4-((2-phenylthio-6-(phenyl)pyridin-3-yl)methoxy)phenoxy)ethanoic acid; 2-(4-(((2-methoxy-5-phenylpyridin-3-yl)methyl)amino)phenylthio)-ethanoic acid; 2-(4-(((2-methoxy-6-phenylpyridin-3-yl)methyl)amino)phenylthio)-2,2-difluoroethanoic acid; 2-(4-(((2-methoxy-5,6-diphenylpyridin-3-yl)methyl)amino)phenylthio)-ethanoic acid; 2-(4-(((2-methoxy-5-bromo-6-phenylpyridin-3-yl)methyl)amino)-phenylthio)-ethanoic acid; 2-(4-(((2-methoxy-6-furylpyridin-3-yl)methyl)amino)phenylthio)ethanoic acid; 3-(4-(((2-methoxy-6-furylpyridin-3-yl)methyl)amino)phenyl)propanoic acid; 2-(4-(((2-methoxy-6-phenylpyridin-3-yl)methyl)amino)phenylthio)-2-phenyl-ethanoic acid; 3-(4-(((2-methoxy-6-phenylpyridin-3-yl)methyl)(methyl)amino)phenyl)-propanoic acid; 3-(4-(1-((2-methoxy-6-phenylpyridin-3-yl)propyl)amino)phenyl)propanoic acid; 2-(4-(((2-methoxy-6-phenylpyridin-3-yl)methyl)amino)-2,6-dimethyl-phenoxy)ethanoic acid; 3-(4-(((2-methoxy-6-(4-(trifluoromethyl)phenyl)pyridin-3-yl)methyl)amino)-phenyl)-propanoic acid; 3-(4-((2-methoxy-6-phenylpyridin-3-yl)methylthio)phenyl)propanoic acid; 3-(4-(((2-(ethylthio)-6-phenylpyridin-3-yl)methyl)amino)phenyl)-propanoic acid; 3-(4-(((2-methoxy-6-(parabiphenyl)pyridin-3-yl)methyl)amino)phenyl)-propanoic acid; 3-(4-(((2-methoxy-6-(3-(trifluoromethyl)phenyl)pyridin-3-yl)methyl)-amino)-phenyl)propanoic acid; 3-(4-(((2-methoxy-5-phenylpyridin-3-yl)methyl)amino)phenyl)propanoic acid; 3-(4-((2(-methoxy-6-phenylpyridin-3-yl)methyl)amino)phenyl)-3-phenyl-propanoic acid; 3-(2-methoxy-4-(((2-methoxy-6-phenylpyridin-3-yl)methyl)amino)-phenyl)-propanoic acid; 3-(3-methoxy-4-(((2-methoxy-6-phenylpyridin-3-yl)methyl)amino)-phenyl)-propanoic acid; 3-(4-(((2-methoxy-6-phenylpyridin-3-yl)methyl)amino)phenyl)butanoic acid; 3-(4-(((2-methoxy-5-(4-(trifluoromethyl)phenyl)pyridin-3-yl)methyl)-amino)phenyl)propanoic acid; 3-(4-(((2-methoxy-5-(3-(trifluoromethyl)phenyl)pyridin-3-yl)methyl)-amino)-phenyl)propanoic acid; 3-(4-(((2,6-dimethoxy-5-phenylpyridin-3-yl)methyl)amino)phenyl)-propanoic acid; 3-(4-(((5-(4-chlorophenyl)-2-methoxypyridin-3-yl)methyl)amino)phenyl)-propanoic acid; 3-(4-(((2-methoxy-5-(naphthalen-2-yl)pyridin-3-yl)methyl)amino)phenyl)-propanoic acid; 3-(4-(((2-ethoxy-6-phenylpyridin-3-yl)methyl)amino)phenyl)propanoic acid; 3-(4-((2-methoxy-5-phenylpyridin-3-yl)methoxy)phenyl)hex-4-ynoic acid; 3-(4-((2-methoxy-6-phenylpyridin-3-yl)methoxy)phenyl)hex-4-ynoic acid; and 3-(4-(((2-isopropyloxy-6-phenylpyridin-3-yl)methyl)amino)phenyl)-propanoic acid, are described in Masson and Caumont-Bertrand, PPAR Agonist Compounds, Preparation and Uses, US 2011/0195993, which is hereby incorporated by reference in its entirety.

According to some embodiments, the therapeutic compound disclosed herein modulates a PPARα nuclear receptor or PPARα signaling pathway by, e.g., at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%.

According to some embodiments, the therapeutic compound may be a PPARα antagonist. Examples include, without limitation, N-((2S)-2-(((1Z)-1-Methyl-3-oxo-3-(4-(trifluoromethyl)phenyl)prop-1-enyl)amino)-3-(4-(2-(5-methyl-2-phenyl-1,3-oxazol-4-yl)ethoxy)phenyl)propyl)propanamide (GW6471, Sigma, 1050=0.24 μM, which enhances the binding affinity of the PPARα ligand-binding domain to the co-repressor proteins SMRT and NCoR) or a derivative thereof.

According to some embodiments, the therapeutic compound may be an FXR agonist. Examples of FXR agonists include, without limitation, GW4064, chenodoxycholic acid (CDCA) and obeticholic acid. According to some embodiments, the therapeutic compound modulates an FXR signaling pathway by, e.g., at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%.

According to some embodiments, the therapeutic compound may be an LXR agonist. Examples of LXR agonists include, without limitation, GW3965. According to some embodiments, the therapeutic compound modulates an LXR signaling pathway by, e.g., at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%.

Bile acids act as signaling molecules through the bile acid-dedicated G-protein coupled receptor TGR5. Stimulation of the TGR5 signaling pathway confers to bile acids the ability to modulate energy expenditure by controlling the activity of type 2 deiodinase and the subsequent activation of thyroid hormone in brown adipose tissue and muscle. This activation triggers an increase in energy expenditure and attenuates diet-induced obesity. Thomas, C. et al, “TGR5-mediated bile acid sensing controls glucose homeostasis,” Cell Meta. 10(3): 167-77 (2007).

According to some embodiments, the therapeutic compound modulates a TGR5 nuclear receptor or a TGR5 nuclear receptor signaling pathway. Exemplary TGR5 pathway agonists include, without limitation, 3-(2-chlorophenyl)-N-(4-chlorophenyl)-N,5-dimethyl-4-isoxazolecarboxamide, CAS 1197300-24-5 (Cayman Chemical Co., 16291). According to some embodiments, the therapeutic compound modulates a TGR5 signaling pathway by, e.g., at least 5%, at least 10%, at least 15%, at last 20%, at least 25%, at least 30%, at last 35%, at least 40%, at least 45%, at least 50%, at least 55%, at least 60%, at least 65%, at least 70%, at least 75%, at least 80%, at least 85%, or at least 90%.

According to some embodiments, modulation of the methyltransferase activity may be effective against tumors comprising a population of tumor cells, e.g., to suppress proliferation of the population of tumor cells, to reduce tumor growth, and/or to reduce tumor burden in a human cancer. Examples of human cancers include, without limitation, leukemias (a cancer that starts in blood-forming tissue), lymphomas (a type of cancer that begins in the immune system), sarcomas (a type of cancer that begins in bone or in the soft tissues of the body, including cartilage, fat, muscle, blood vessels, fibrous tissue, or other connective or supportive tissue. Different types of sarcoma are based on where the cancer forms. For example, an osteosarcoma forms in bone, a liposarcoma forms in fat, and a rhabdomyosarcoma forms in muscle), and carcinomas (a type of cancer that begins in the skin or in tissues that line or cover the internal organs). Nonlimiting examples of human cancers include, without limitation, breast cancer, lung cancer, colorectal cancer, enterohepatic cancer, bladder cancer, mantle cell lymphoma, pancreatic cancer, prostate cancer, thyroid cancer, and brain cancer.

According to some embodiments, modulation of the methyltransferase activity may be effective to induce apoptosis in the population of tumor cells.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials have been described. All publications mentioned herein are incorporated herein by reference to disclose and described the methods and/or materials in connection with which the publications are cited.

It must be noted that as used herein and in the appended claims, the singular forms “a”, “and”, and “the” include plural references unless the context clearly dictates otherwise.

The publications discussed herein are provided solely for their disclosure prior to the filing date of the present application and each is incorporated by reference in its entirety. Nothing herein is to be construed as an admission that the present invention is not entitled to antedate such publication by virtue of prior invention. Further, the dates of publication provided may be different from the actual publication dates which may need to be independently confirmed.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Compounds that affect bile acids, glucose metabolism and nuclear receptors were screened to identify any measurable effects against protein arginine methyltransferases PRMT1 (Genbank Accession No. NM_001536, SEQ ID NO: 1), PRMT5 (Genbank Accession No. NM_006109; SEQ ID NO: 2), PRMT5 (Genbank Accession No. NM_006109, SEQ ID NO: 3), /MEP50 (Genbank Accession NO. NM_024102; SEQ ID NO: 4), and PRMT6 (Genbank Accession No. NM_018137; SEQ ID NO: 5). The screen included two PPAR agonists, a TGR5 agonist, an LXR agonist, an FXR agonist, metformin, chenodeoxycholic acid (CDCA) and obetocholic acid (OBCA).

3-(2-chlorophenyl)-N-(4-chlorophenyl)-N,5-dimethyl-4-isoxazolecarboxamide, CAS 1197300-24-5 (Cayman Chemical Co., 16291), a TGR5 agonist, was supplied as a crystalline solid. It is a synthetic small molecular activator of TGR5, a G-protein coupled receptor for bile acids) (pEC50=6.8-7.5), which is reported to improve glucagon-like peptide 1 (GLP-1) secretion by increasing intracellular cAMP levels via TGR5.

3-[3-[[[2-chloro-3-(trifluoromethyl)phenyl]methyl](2,2-diphenylethyl)amino]propoxy]-benzeneacetic acid, monohydrochloride (GW 3965, CAS 405911-17-3, Cayman Chemical, 10054) was supplied as a crystalline solid. It is an orally active agonist of LXRα and LXRβ, activating the human isoforms with EC50 values of 290 and 30 nM respectively. It alters LXR-regulated gene expression in mice and rats, affecting pathways related to glucose and lipid metabolism and affects inflammation and pressor responses through LXRα and LXRβ.

3-[2-[2-chloro-4-[[3-(2,6-dichlorophenyl)-5-(1-methylethyl)-4-isoxazolyl]methoxy]phenyl]ethenyl]-benzoic acid (GW4064, CAS 278779-30-9, Cayman Chemical 10006611) was supplied as a crystalline solid. It is a selective agonist of FXR (EC50=15 nM). It displays no activity at other nuclear receptors, including the retinoic acid receptor, at concentrations up to 1 μM.

N, N-dimethyl-imidodicarbonimidic diamide, monohydrochloride (Metformin hydrochloride, CAS 1115-70-4, Cayman Chemical 13118) was supplied as a crystalline solid. It is an orally administered biguanide derivative used to lower blood glucose concentrations in patients with non-insulin dependent diabetes mellitus. It improves insulin sensitivity and decreases insulin resistance by inhibiting complex 1 of the mitochondrial respiratory chain and inducing AMP-activated protein kinase-dependent signaling; has been shown to improve fatty liver disease by reversing hepatic steatosis in ob/ob mice, to restore ovarian function in polycystic ovary syndrome; and to directly inhibit tumor growth in cancer cell lines.

5-[[4-[2-(5-ethyl-2-pyridinyl)ethoxy]phenyl]methyl]-2,4-thiazolidinedione (Pioglitazone, CAS 111025-46-8, Cayman Chemical, 71745) was supplied as a crystalline solid. Piaglitazone is a thiazolidinedione. Thiazolidinediones (TZDs) are a group of structurally related peroxisome proliferator-activated receptor γ (PPARγ) agonists with antidiabetic actions in vivo. Pioglitazone selectively activates PPARγ-1. Pioglitazone is about one tenth as potent as rosiglitazone, with an EC50 of about 500-600 nM for both human and mouse PPARγ.

5-[[4-[2-(methyl-2-pyridinylamino)ethoxy]phenyl]methyl]-2,4-thiazolidinedione (Rosiglitazone, CAS 122320-73-4, Cayman Chemical, 71740) was supplied as a crystalline solid. It is a prototypical TZD and has served as a reference compound for this class of PPARγ ligands. In a transactivation assay using COS-1 cells transfected with full length human PPARα and RXRα, pioglitazone and rosiglitazone exhibit low level activation of PPARα at 1 μM and 5.4- and 4.2-fold activation, respectively, at a concentration of 10 μM.

3α, 7α-dihydroxy-5β-cholan-24-oic acid (Chenodeoxycholic acid, or CDCA, CAS 474-25-9, Cayman Chemical 10011286) was supplied as a crystalline solid. Chenodeoxycholic acid (CDCA) is a hydrophobic primary bile acid that activates nuclear receptors involved in cholesterol metabolism. EC50 concentrations for activation of FXR range from 13-34 μM. In cells, CDCA also binds to bile acid binding proteins (BABP) with a reported stoichiometry of 1:2. Exposure of cells to excess CDCA contributes to liver and intestinal cancers.

6α-Ethyl-chenodeoxycholic acid (6-α-ECDC; Obeticholic acid; INT-747, AdipoGen, AG-CR1-3560-M005) was supplied as a white solid. It is a potent and selective FXR agonist (EC50=99 nM. It induces SHP in HSCs to suppress TIMP-1 expression, is an apoptosis inducer; protects against liver fibrosis development in rat in vivo; displays anticholeretic activity in rats in vivo; promotes preadipocyte differentiation; regulates adipogenesis and insulin signaling in vivo, and inhibits vascular smooth muscle cell inflammation and migration.

A 10 mM DMSO stock solution of each test compound was prepared. 30-50 μL aliquots were used for assay and the remainder kept in a −80 degree C. freezer:

Compounds were tested in a single-dose mode, in duplicate at the following concentrations.

| Starting concentration | 100 μM | 10 μM | 1 μM |
|---|---|---|---|
| Dose | 3-fold dilution | 3-fold dilution | 3-fold dilution |
| 1 | 1.00E−04 | 1.00E−05 | 1.00E−06 |
| 2 | 3.33E−05 | 3.33E−06 | 3.33E−07 |
| 3 | 1.11E−05 | 1.11E−06 | 1.11E−07 |
| 4 | 3.70E−06 | 3.70E−07 | 3.70E−08 |
| 5 | 1.23E−06 | 1.23E−07 | 1.23E−08 |
| 6 | 4.12E−07 | 4.12E−08 | 4.12E−09 |
| 7 | 1.37E−07 | 1.37E−08 | 1.37E−09 |
| 8 | 4.57E−08 | 4.57E−09 | 4.57E−10 |
| 9 | 1.52E−08 | 1.52E−09 | 1.52E−10 |
| 10 | 5.08E−09 | 5.08E−10 | 5.08E−11 |

Control Compound, SAH (S-(5′-Adenosyl)-L-homocysteine) was tested in 10-dose IC50 mode with 3-fold serial dilution starting at 100 μM as shown. SAH is an inhibitor of SAM-dependent methyltransferases (positive control). Reactions were carried out at 1 μM SAM. The no inhibitor control was set as 100% activity).

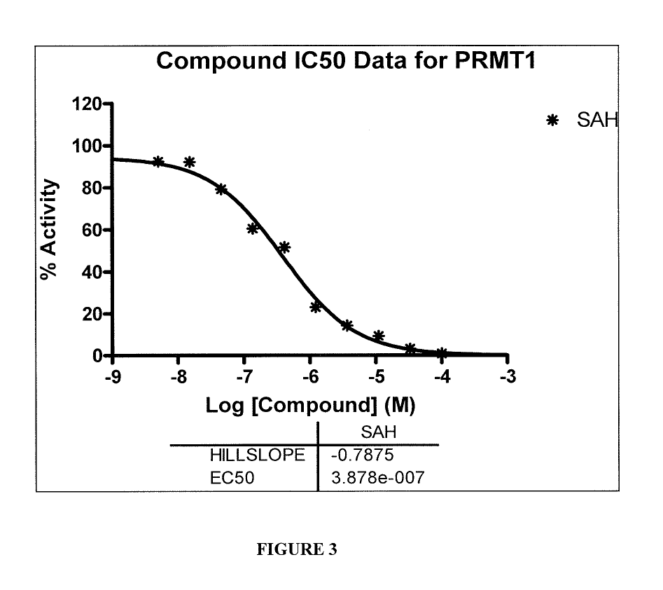

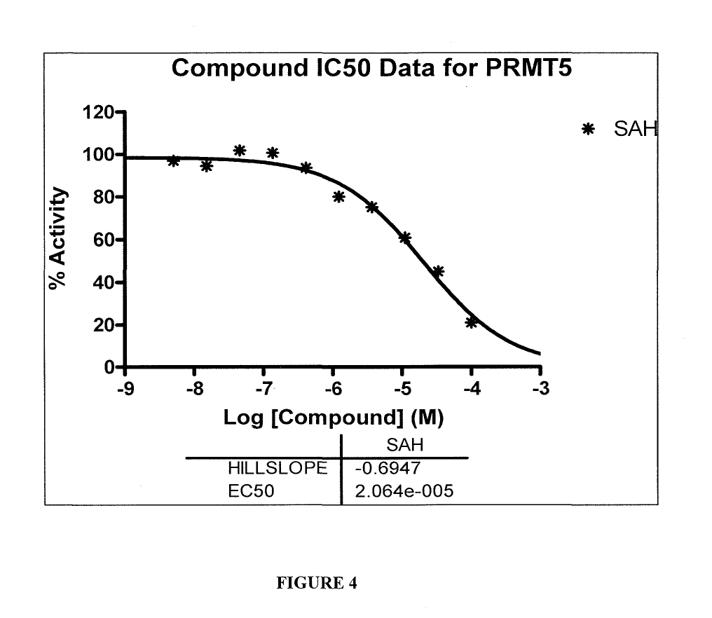

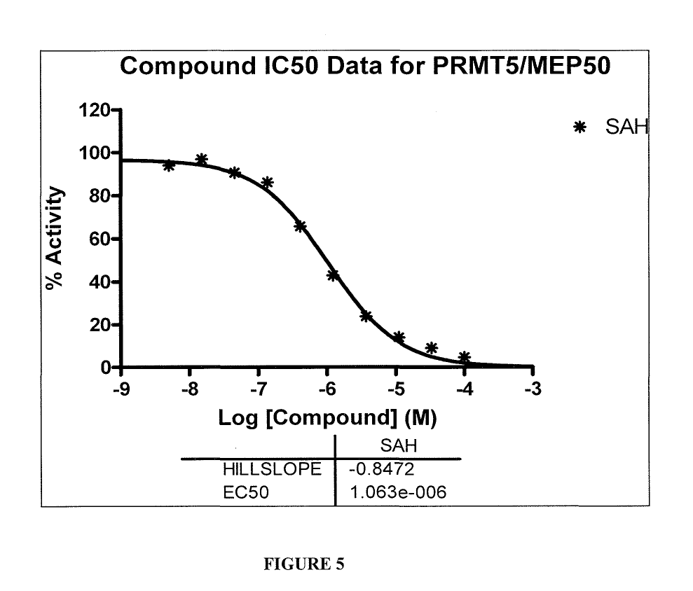

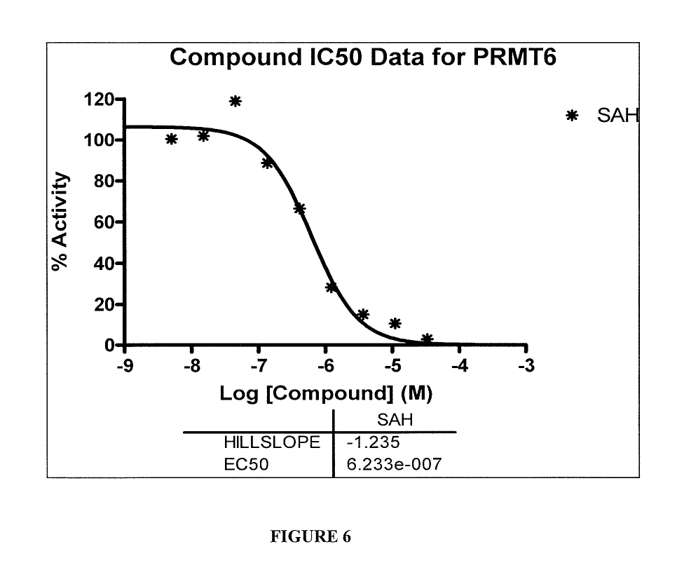

Results of the assay are shown in Table 3. IC50 curves for SAH and PRMT1, PRMT5, PRMT5/MEP50 and PRMT6 SAH are shown in FIGS. 3, 4, 5 and 6 respectively.

Differential effects on PRMT5 were observed between pioglitazone (PIO, sold commercially as Actos®) and rosiglitazone (ROSI, sold commercially as Avandia®). PIO is a mild inhibitor of PRMT5 at a concentration of 100 μM, while ROSI had no effect.

Chenodeoxycholic acid had no direct inhibitory effect on PRMTs.

TABLE 3
Single Dose Enzyme Activity
Summary:

| | | | % Enzyme Activity (relative to DMSO controls) | | | |
|---|---|---|---|---|---|---|
| | | Methyltransferase: | PRMT1 | PRMT5 | PRMT5/MEP50 | PRMT6 |
| | Compound ID: | Substrate: | Histone H4 | Histone H2A | Histone H2A | Histone H3 |
| 1 | INT-747 | Data 1 | 112.72 | 106.35 | 95.73 | 312.24 |
| | | Data 2 | 115.42 | 105.51 | 93.91 | 288.93 |
| 2 | Pioglitazone | Data 1 | 110.44 | 18.95 | 43.68 | 93.46 |
| | | Data 2 | 99.23 | 18.46 | 45.13 | 102.78 |
| 3 | Rosiglitazone | Data 1 | 107.80 | 50.35 | 77.90 | 106.27 |
| | | Data 2 | 99.72 | 50.35 | 77.42 | 99.47 |
| 4 | Metformin | Data 1 | 100.50 | 98.74 | 88.20 | 90.66 |
| | | Data 2 | 101.26 | 84.85 | 82.84 | 90.30 |
| 5 | TGR5 agonist | Data 1 | 100.02 | 84.41 | 90.97 | 70.12 |
| | | Data 2 | 92.67 | 87.02 | 82.32 | 73.79 |
| 6 | Chenodeoxycholic | Data 1 | 117.52 | 83.22 | 88.22 | 115.95 |
| | acid | Data 2 | 96.44 | 86.95 | 93.72 | 121.09 |
| 7 | GW 4064 | Data 1 | 14.82 | 102.42 | 1.50 | 120.60 |
| | | Data 2 | 10.85 | 94.01 | 1.18 | 173.14 |
| 8 | GW 3965 | Data 1 | 6.93 | 90.29 | 1.54 | 83.29 |
| | | Data 2 | 4.55 | 97.35 | 1.23 | 103.66 |
| | SAH IC50* (M): | | 3.88E−07 | 2.06E−05 | 1.06E−06 | 6.23E−07 |

### Example 2

TABLE 4
Methyltransferase Profiling Report
for: PRMT5/MEP50

| | | | | |
|---|---|---|---|---|
| Conc. (M) | GW 4064 | GW 3965 | Conc. (M) | SAH |

Raw data

| | | | | |
|---|---|---|---|---|
| 1.00E−04 | 24954 | 6431 | 1.00E−04 | 23637 |
| 3.33E−05 | 527528 | 602561 | 3.33E−05 | 51347 |
| 1.11E−05 | 515444 | 544281 | 1.11E−05 | 102087 |
| 3.70E−06 | 453113 | 494019 | 3.70E−06 | 183946 |
| 1.23E−06 | 463109 | 463366 | 1.23E−06 | 268933 |
| 4.12E−07 | 435450 | 453763 | 4.12E−07 | 319517 |
| 1.37E−07 | 437127 | 454053 | 1.37E−07 | 384445 |
| 4.57E−08 | 425743 | 434862 | 4.57E−08 | 424069 |
| 1.52E−08 | 424387 | 417100 | 1.52E−08 | 394475 |
| 5.08E−09 | 414110 | 433085 | 5.08E−09 | 401476 |
| DMSO | 439696 | 444461 | DMSO | 408103 |

% Activity

| | | | | |
|---|---|---|---|---|
| 1.00E−04 | 5.70 | 1.47 | 1.00E−04 | 5.40 |
| 3.33E−05 | 120.51 | 137.65 | 3.33E−05 | 11.73 |
| 1.11E−05 | 117.75 | 124.34 | 1.11E−05 | 23.32 |
| 3.70E−06 | 103.51 | 112.86 | 3.70E−06 | 42.02 |
| 1.23E−06 | 105.80 | 105.86 | 1.23E−06 | 61.44 |
| 4.12E−07 | 99.48 | 103.66 | 4.12E−07 | 72.99 |
| 1.37E−07 | 99.86 | 103.73 | 1.37E−07 | 87.83 |
| 4.57E−08 | 97.26 | 99.34 | 4.57E−08 | 96.88 |
| 1.52E−08 | 96.95 | 95.29 | 1.52E−08 | 90.12 |
| 5.08E−09 | 94.60 | 98.94 | 5.08E−09 | 91.72 |
| DMSO | 100.45 | 101.54 | DMSO | 93.23 |
| HILLSLOPE | −12.23 | −24.99 | | −0.78 |
| IC50 (M) | 7.92E−05 | 8.42E−05 | | 2.71E−06 |

*Data excluded from curve fit

These data show that GW4064 and GW3965, the two test compounds that hit PRMT1:5, have nanomolar affinity for LXR and FXR, but macromolar affinity 80-80 for PRMT5/MEP50.

### Example 3

### Inhibition of Cancer Cell Proliferation

A cell proliferation assay can be employed to determine inhibition of cancer cell proliferation by the test compounds of Example 1. For example, cancer cell lines such as CaCo2 (colon), PC-3 (prostate), MCF7 (breast), MeLa (cervical), TOV-112D (ovarian), NCI-H69 (small cell lung), A 549 (non-small cell lung), SK-MEL-24 (melanoma), SNF96.2 (nerve/Schwann cell), HT-1376 (bladder), A498 (kidney), HEPG2 (liver), PANC-1 (pancreas) and AGS (stomach) can be obtained from ATCC, seeded in 96-well plates, and cultured overnight. Cell cultures can be exposed to various concentrations of the test compounds for specified treatment intervals. Proliferation of cancer cells can be determined by 3-[4,5-dimethylthiazol-2-yl]-2,5-diphenyltetrazolium bromide (MTT) assay (Life Technologies, Grand Island, N.Y.). Briefly, for adherent cells, medium is removed and replaced with 100 μL of fresh culture medium. For non-adherent cells, microplates are centrifuged to pellet cells, the medium is removed and replaced with 100 μL of fresh medium. MTT dye (2 mg/ml) is added, and the cells are incubated for 4 hours at 37° C. Next, medium is removed and the resulting formazan crystals are dissolved in DMSO (Sigma-Aldrich, St. Louis, Mo.) for 5 minutes. Microplates are read in a spectrophotometer at 540 nm. Dose response curves can be created using GraphPad Prism version 5.01 (GraphPad Software Inc, La Jolla, Calif.). IC₅₀ values can be calculated using CalcuSyn (Biosoft, Great Shelford, Cambridge, UK).

### Example 4

### Cytotoxicity

The cytotoxic effects of the test compounds of Example 1 on cancer cells can be determined. For example, cancer cell lines such as CaCo2 (colon), PC-3 (prostate), MCF7 (breast), MeLa (cervical), TOV-112D (ovarian), NCI-H69 (small cell lung), A549 (non-small cell lung), SK-MEL-24 (melanoma), SNF96.2 (nerve/Schwann cell), HT-1376 (bladder), A498 (kidney), HEPG2 (liver), PANC-1 (pancreas) and AGS (stomach) can be obtained from ATCC, seeded in 6-well plates and cultured overnight. Cell cultures can be left untreated or treated with predetermined IC₅₀ doses of the test compounds and incubated for 48, 72 or 96 hours. After each time point, media containing any non-adherent cells can be collected. Adherent cells can be detached using trypsin (Life Technologies, Grand Island, N.Y.), suspended in culture medium and disaggregated by manual pipetting. All collected cells can be mixed with trypan blue dye (Life Technologies). Viable cells that exclude the dye and dead cells that stain blue can be counted using a hemocytometer.

### Example 5

### Apoptosis

Activation of caspase enzymes is a distinctive feature of the early stages of apoptosis. A microplate assay for caspase activity can be performed to determine apoptosis of cancer cells by INT-747, GW4064 and GW3965. For example, cancer cell lines such as CaCo2 (colon), PC-3 (prostate), MCF7 (breast), MeLa (cervical), TOV-112D (ovarian), NCI-H69 (small cell lung), A549 (non-small cell lung), SK-MEL-24 (melanoma), SNF96.2 (nerve/Schwann cell), HT-1376 (bladder), A498 (kidney), HEPG2 (liver), PANC-1 (pancreas) and AGS (stomach) can be obtained from ATCC, seeded in 96-well plates and cultured overnight. Cell cultures can be left untreated or treated with predetermined IC₅₀ doses of the test compounds and incubated for 48, 72 or 96 hours. After each time point, CellEvent™ Caspase-3/7 Green Detection Reagent (Life Technologies) can be added to each well at a final concentration of between 2-8 μM and incubated at 37° C. for 30 minutes. After incubation, cells can be imaged using a microplate reader with fluorescence detection (e.g., Gemini XPS Microplate Reader, Molecular Devices, Sunnyvale, Calif.) with filter sets capable of detecting 502/530 nm excitation/emission maxima.

### Example 6

### In Vivo Treatment of Cancer Cells

A mouse xenograft model can be used to evaluate the effect of the test compounds on tumor cells in vivo. For example, cancer cell lines such as CaCo2 (colon), PC-3 (prostate), MCF7 (breast), MeLa (cervical), TOV-112D (ovarian), NCI-H69 (small cell lung), A549 (non-small cell lung), SK-MEL-24 (melanoma), SNF96.2 (nerve/Schwann cell), HT-1376 (bladder), A498 (kidney), HEPG2 (liver), PANC-1 (pancreas) and AGS (stomach) can be obtained from ATCC and cultured in standard cell culture medium. Tumors can be induced in 8-week-old female anaesthetized (mixture of 2-3% isoflurane in O₂ at a flow of 0.6 L/min) BALB/cOlaHSD-Foxn1^{nu} nude mice (Harlan Laboratories) by inoculating approximately 1 million cancer cells suspended in 0.1 mL of 50% Matrigel™ (BD Biosciences) in phosphate-buffered saline (PBS, Gibco) intradermally at the right flank of each mouse. Tumor growth can be continuously monitored by measuring the tumor width and length 2-3 times per week with a caliper, where length is defined as the longest side and width is defined as the perpendicular to the length. The measurement can be carried out by a single operator over the entire study period for consistency. Tumor volume (mm³) can be calculated by the equation:

Volume=length×(width)²/2 [Equation 1]

Once the tumor reaches about 80 mm³ (approximately day 25 post inoculation), mice can be divided into the following groups: untreated control group; and a group for each test compound. Mice can be orally treated once a day for 6 days/week. After 30 days of oral treatment, mice can be sacrificed and tumors can be collected.

Drug anti-tumor activity can be evaluated as the tumor growth inhibition percentage calculated by the 1-treated/control tumor volume ratios (1-T/C) (Johnson J I et al., British Journal of Cancer 1998; 84: 1424-1431; Hollingshead M G, Journal of the National Cancer Institute 2008; 100: 1500-1510). Data can be expressed as the median with the minimum-maximum value interval.

While the present invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present invention. All such modifications are intended to be within the scope of the claims appended hereto.

What is claimed is:

1. A method for modulating gene expression of a gene related to proliferation of a population of tumor cells comprising administering a therapeutic amount of a therapeutic compound to a cell, a tissue, or a mammal, wherein the therapeutic amount of the therapeutic compound is effective to modulate a methyltransferase activity of a protein arginine methyltransferase, which in turn modulates methylation of a target protein that affects gene expression of the gene, and may suppress the proliferation of the population of tumor cells.

2. The method according to claim 1, wherein the target protein is a histone.

3. The method according to claim 2, wherein the histone is one or more of histone H2A, histone H3, or histone H4.

4. The method according to claim 1, wherein the protein arginine methyltransferase (PRMT) is one or more of protein arginine methyltransferase 1 (PRMT1), protein arginine methyl transferase 5 (PRMT5), protein arginine methyltransferase 5 complexed with MEP50 (PRMT5/MEK50), or PRMT6.

5. The method according to claim 1, wherein modulating the methyltransferase activity of the protein arginine methyltransferase (PRMT) comprises modulating a nuclear receptor or a nuclear receptor signaling pathway.

6. The method according to claim 5, wherein modulating the methyltransferase activity of the protein arginine methyltransferase (PRMT) comprises modulating a nuclear receptor or a nuclear receptor signaling pathway by at least 5%, at least 10%, at least 15%, at last 20%, at least 25%, at least 30%, at last 35%, at least 40%, at least 45%, at least 50%, compared to a control.

7. The method according to claim 6, wherein the nuclear receptor is one or more of a PPAR nuclear receptor, an FXR nuclear receptor, or an LXR nuclear receptor.

8. The method according to claim 6, wherein the nuclear receptor may comprise a nuclear receptor heterodimer complex.

9. The method according to claim 7, wherein the PPAR receptor, the FXR nuclear receptor, or the LXR nuclear receptor is complexed with a retinoid X receptor (RXR).

10. The method according to claim 9, wherein the therapeutic compound is a PPAR ligand, an FXR ligand, an LXR ligand, or an RXR ligand.

11. The method according to claim 10, wherein binding of the therapeutic compound to the PPAR-RXR, FXR-RXR, or LXR-RXR heterodimer complex may modulate binding of the complex to responsive elements in target DNA.

12. The method according to claim 6, wherein the therapeutic compound modulates a PPARγ signaling pathway.

13. The method according to claim 6, wherein the therapeutic compound is a PPAR agonist that modulates a PPAR nuclear receptor or a PPAR signaling pathway.

14. The method according to claim 13, wherein the PPAR agonist is a PPAR-γ agonist selected from Rosiglitazone, a derivative of Rosiglitazone, Pioglitazone, and a derivative of Pioglitazone.

15. The method according to claim 6, wherein the therapeutic compound is a PPAR-α antagonist that modulates a PPARα nuclear receptor or PPARα signaling pathway.

16. The method according to claim 6, wherein the therapeutic compound is an FXR agonist that modulates an FXR nuclear receptor or an FXR signaling pathway.

17. The method according to claim 16, wherein the FXR agonist is GW4064 a derivative of GW4064, obeticholic acid, or a derivative of obeticholic acid.

18. The method according to claim 6, wherein the therapeutic compound is an LXR agonist that modulates an LXR receptor or an LXR signaling pathway.

19. The method according to claim 18, wherein the LXR agonist is GW3965 or a derivative thereof.

20. The method according to claim 6, wherein the therapeutic compound is a TGR5 agonist that modulates a TGR5 signaling pathway.

21. The method according to claim 20, wherein the TGR agonist is 3-(2-chlorophenyl)-N-(4-chlorophenyl)-N,5-dimethyl-4-isoxazolecarboxamide (TGR5) or a derivative thereof.

22. The method according to claim 1, wherein the method may be effective to reduce tumor growth, and/or to reduce tumor burden in a human cancer.

23. The method according to claim 22, wherein the population of tumor cells is a population of leukemia tumor cells, lymphoma tumor cells, sarcoma tumor cells, carcinoma tumor cells, breast tumor cells, lung tumor cells, colorectal tumor cells, enterohepatic tumor cells, bladder tumor cells, mantle cell tumor cells, pancreatic tumor cells, prostate tumor cells, thyroid tumor cells, or brain tumor cells.

24. The method according to claim 1, wherein the therapeutic amount of the therapeutic compound may be effective to induce apoptosis in the population of tumor cells.
